SEQUENCE LISTING (120 sequences)

SEQ ID NO: 1 - DNA, 4347 bp, Homo sapiens
tgaccgcgtgccgggtgtcatggcggcctgcaggtactgctgctcgtgcctccggctccggcccctgagcgatggtcctttccttctgccacggcgggatcgggcactcacccagttgcaagtgcgagcactatggagtagcgcagggtctcgagctgtggccgtggacttaggcaacaggaaattagaaatatcttctggaaagctggccagatttgcagatggctctgctgtagtacagtcaggtgacactgcagtaatggtcacagcggtcagtaaaacaaaaccttccccttcccagtttatgcctttggtggttgactacagacaaaaagctgctgcagcaggtagaattcccacaaactatctgagaagagaggttggtacttctgataaagaaattctaacaagtcgaataatagatcgttcaattagaccgctctttccagctggctacttctatgatacacaggttctgtgtaatctgttagcagtagatggtgtaaatgagcctgatgtcctagcaattaatggcgcttccgtagccctctcattatcagatattccttggaatggacctgttggggcagtacgaataggaataattgatggagaatatgttgttaacccaacaagaaaagaaatgtcttctagtactttaaatttagtggttgctggagcacctaaaagtcagattgtcatgttggaagcctctgcagagaacattttacagcaggacttttgccatgctatcaaagtgggagtgaaatatacccaacaaataattcagggcattcagcagttggtaaaagaaactggtgttaccaagaggacacctcagaagttatttaccccttcgccagagattgtgaaatatactcataaacttgctatggagagactctatgcagtttttacagattacgagcatgacaaagtttccagagatgaagctgttaacaaaataagattagatacggaggaacaactaaaagaaaaatttccagaagccgatccatatgaaataatagaatccttcaatgttgttgcaaaggaagtttttagaagtattgttttgaatgaatacaaaaggtgcgatggtcgggatttgacttcacttaggaatgtaagttgtgaggtagatatgtttaaaacccttcatggatcagcattatttcaaagaggacaaacacaggtgctttgtaccgttacatttgattcattagaatctggtattaagtcagatcaagttataacagctataaatgggataaaagataaaaatttcatgctgcactacgagtttcctccttatgcaactaatgaaattggcaaagtcactggtttaaatagaagagaacttgggcatggtgctcttgctgagaaagctttgtatcctgttattcccagagattttcctttcaccataagagttacatctgaagtcctagagtcaaatgggtcatcttctatggcatctgcatgtggcggaagtttagcattaatggattcaggggttccaatttcatctgctgttgcaggcgtagcaataggattggtcaccaaaaccgatcctgagaagggtgaaatagaagattatcgtttgctgacagatattttgggaattgaagattacaatggtgacatggacttcaaaatagctggcactaataaaggaataactgcattacaggctgatattaaattacctggaataccaataaaaattgtgatggaggctattcaacaagcttcagtggcaaaaaaggagatattacagatcatgaacaaaactatttcaaaacctcgagcatctagaaaagaaaatggacctgttgtagaaactgttcaggttccattatcaaaacgagcaaaatttgttggacctggtggctataacttaaaaaaacttcaggctgaaacaggtgtaactattagtcaggtggatgaagaaacgttttctgtatttgcaccaacacccagtgttatgcatgaggcaagagacttcattactgaaatctgcaaggatgatcaggagcagcaattagaatttggagcagtatataccgccacaataactgaaatcagagatactggtgtaatggtaaaattatatccaaatatgactgcggtactgcttcataacacacaacttgatcaacgaaagattaaacatcctactgccctaggattagaagttggccaagaaattcaggtgaaatactttggacgtgacccagccgatggaagaatgaggctttctcgaaaagtgcttcagtcgccagctacaaccgtggtcagaactttgaatgacagaagtagtattgtaatgggagaacctatttcacagtcatcatctaattctcagtgattttttttttttaaagagaattctagaattctattttgtctagggtgatgtgctgtagagcaacattttagtagatcttccattgtgtagatttctatataatataaatacattttaattatttgtactaaaatgctcatttacatgtgccatttttttaattcgagtaacccatatttgtttaattgtatttacattataaatcaagaaatatttattattaaaagtaagtcatttatacatcttagaaaaaattacatagttttgtttttacaattctgaaatatatgaaaaacttagatagaatatgtcatatgttattataacagatctcttcctacatcttatttttcttctttgtatacagtagtgacagtttaccttcagtcatcctacaggtcacctctgtgcccaacaactttacttgcctacaacggttcacaaactggaacaggccatattcaaagccagtgcctatttcttcagaactgttaacagatatagtgagttgagggagctaatctgatacacttttgataatataatgcctttcaaattagttaccaaatcataaacagagtggaataaatataaatgagattctaactaggatgaatgtggtagtaatgatgtatatttttcaaatttacctaaacagaaagattattggctaaggcaggcggatcatgagatcaggagatcgagaccatcctggctaacacggtgaaaccccgtctctactaaaagtgcaaaaattagccgggcgtggtggcgcatgcctgtaatcctagctactcaggaggctgaggcagaagaatcacttcaacccaggaggtggaggttgtagtgagccgagattgcgccattgcactccagctcaggcaacaagagcaaaactccgtctcaaaaaaaaaaaaaaaattgtttacttatcaaattgaactaggtcaatttgaatatgtgtattgctctaatgtatggaagttcttagtataagagctgaaggatcatgactttcggttaatagatgtttgaaatagatgtttagaggttactgatacacatgtcaagttgttcattcccatcatttaaaatatcaccttataattccacagaaaagtaaatggtatgtggacgtcataatttagaatgtgtaatcttttttaaaatcttataaaaatacactggactcagtggtacgtgcctgtagtcccagctacttggaaggctgaagtgggaaggtcactagagcccagaagttcgacactgtagtgtgcaacaatcatacctgtgaatagctactgtactccagcctgggcaacatagcaggactcttatctctaaaaaaaattttaaatcttataaaaatgtaggtatatctggccaggcatggtggctcacacctgtaatcccaacactttgggaggtcaaggcgggtagatcacctgaggtcaggagttcgagaccagcctggccaacatggtgaaaccccatctctactaaaaatacaaaaattagctgggtgtggtggcgggcgcctgtaatcccagctactcaggaggctgaggcaggagaatctcttgaacctgggaggcagaggtggcagtgagctgagatcgcaccactgcactccagcctgggcaaaaagagtaaaactccatctcaaaaaaaaaaatggtgggcacagaagctcatgcctgtaattccagcactttgggaggccgaggcgggtggattacatgaggtcaggaattcaagaccagcctggccaacacggtaaaaccccatctctactaaaaatacaaaaattagctgggcatggtggcaggcacctgtaatcctagctactcgggaggctgaggcaggagaatcacttgagcctgggaaatggaggttgcagtgagccgagattgcgccattgcactccagcctgggcgacagagcaagactccgtctccagaaaaaaaaaaaaaaaa

SEQ ID NO: 2 - protein, 783 aa, Homo sapiens [sequence omitted]

SEQ ID NO: 3 - DNA, 410 bp, Homo sapiens
aggcccaccacccaaattctaattcttagttcacagatccaaataataataatgtgttgttctatgacttctagtattaaaagtaaatataaatttactctgcatattaaacatttacattattctaggacttctcctttaaattacgatagtacacacacaaaaaccttatttatctaattgcctttcgttggcacataaagacaaagatcgtttgcaacataatcgcgttctgatacgtttgaccctctctagtcttcgtagaaggtggccaaggtttccgttaccccggcgtccctcgggcaccgcggaaacgaaactccatcaggctccgccccacggtctgcggagtgagccaatcagggcacagcctgcgttgaccgcgtgccgggtgtcatggcggcctgcag

SEQ ID NO: 4 - DNA, 4052 bp, Homo sapiens
cttatagtctatacaaagcagtttaaattttatccttaaagatttaggaagccactgaaaaaatttaatcaggatggagacacaattctatttgtgattgagaaagaagatccttctggtagtggtgtagcacagactttcttaaagttgagtaggctacattctttcaagagaagaattttttcctgaacacttcagggactgctgatacttttaattataatgttactatataacaaaatataacttccatattgttacagcttttagtcataaaagcaatataaatgtatatcctgtctaaactaaactacaaactaaattaaaatatagttaaagggccgggctctgtggctcacgcctgtaatcccagcattttgtgaggccaaggcaggcagatcacctgagggtgggagttggagatcagcctggccaacatggtgaaaccctgtctctattaaaagtacaaaaattagctgggcgcggtggcgcacgtctgtagtcccagctacttgggaggctgaggtaggagaattgcttgaacccaggaggtggaggttgcagtgagccaagattgtgccactgcactccagcttgggcaacagagcaagactccatctcaaaaaaaaaaaaagtagttaagcataatttaatgcaatggtgttttacttaacgtagttgcttattcagaacatgcagtggtattaactgttttgatgcaggttcatcagagtttgaagaaaagggaaaaggagatctgagaagttgctggtggatttaagttggatcatttttaatgtccttcagcagtcataggtaaataaatcccttttaaattatattttggctgttgaactacacttgacataactctacttgcagaataacatctcagactgaacctcacggattatagcttcagtagtaacaactacaagaatttaaacttattatatttttctttctacgctaatctaaaaatgcttaatataaacatataaacgcattctgtatcatctatatcttcataataaagctctgctgcaagattttagtgcctacatttgcttttgagtttactatccaattactactatttgaattatatcagatttgtgtgaggaaatgggggtgggggattcttaataggtaaatgctgctttccatcacttctggctggataaaagctaaccgattcaaggagttcaaggagttctgaagaagagaaaaatgttcagaatacctggaaactggaggaagaattaagaaaagtctggataagcctgggcgcggtggctcatgcctgtaatcccagcactttgggaggccgtggcgggtggattgcctgaggtcaggagttcaagaccaacctggccaacatgatgaaaccccatctctactaaaaatccaaaaaattccccgggttttgtgggcagcccttttactcccaactttggaggggggctgaggggggggaatttgcatcccccccggacgcgggtgttccagtgagctgagatcacgccactgcactctagcctgggcaacaagagcaaaactctgtcttgaaaaaaacgaaaaggctggatgaaagctaaatttaccatttattgagtgttgacaatgtgctaggtactattatatcatctgacacttttttttttttttttttttttgagacggagtctcgctctgtcgcccaggccggactgcggactgcagtggcgcaatctcggctcactgcaagctccgcttcccgggttcacgccattctcctgcctcagcctcccgagtagctgggactacaggcgcccgctaccgcgcccggctaattttttgtatttttagtagagacggggtttcaccttgttagccaggatggtctcaatctcctgacctcatgatccacccgcctcggcctcccaaagtgctaggattacaggcgtgagccaccgcgcccggccgacacttttaatccttacaaaaaccctattagattcatattactatctgtttttatggggaaactgagactcagagagctagagtcacacaactggtaattaacagagacaagattggaaccagctgggactaactccaagtctatgctctaaactattaggcaatactgcccaaatgaaaatgattacatagtaattaagtatattaacaaaaagatgattttgtattcttactctataaatcaaaagtcatgttcaattgtggcaagtaggttttctgagagatttatcttcatgagagatttcaatagattggtctggaaaactctgtggcaactcaaggtaaagtaaaagggaggagacgcaaaaactgctttaacactaaaaatctgtaaacaaagttaatggtaatccctctattggaatggaaatcccaaatcagtaataatagctcatgaaagcaaggtgtcagaaaactgctttgccagcaaatcatttgtctgtttagagctcatcattcttttttcttttttaaatcatcacggatcaacatttttaattttttttttttttttttttttttttagatggagtctcggtctggagtgcagtggcgcgatctcggctcactgcaacctccgcctcctggattcaagcgattctcatgcctcagcctctggagtagctgggattacaggcacgcgccaccacaccgagctaatttttgtatttttagtagagacgcggtttcagcatgttggccaggatgccctgggtctcttgacctcgtgatccacccgcctctgcctcccagcgtgctgggattacaggcatgagccactgcgcccggacttttttttttttttttttttttttttttttttttttgagaccgagtttcactcttgttgcaatgacgcgatctcggctcactgcaacctccgcctcccgggttcaagcgattcccctgcctcagcctcccgagtagctgggattacaggcataagccaccatgcccggctagttttgtatttttagtagagacggggtttctctgtgttggtcaggctggtctcgaactcaggtgatccgcccgcctcggcctcccaaagtgctgggattacaggcgtgagccacagcgtccagccaacgtttttaattcttaaaactgtgtttgaatgagtgttgggtttttgttgtttggttttctttggggtaaggcggttatcaccttacacaaaaaatttacatcataatatgaaaatgcccattggttcattcaaggaaaaacataaagaatttctaacagaatggattaatggggtggaagaaatttatccattctctaaaaatttttcaaatcccaaaaacttttttttttcattttacaaaatatttacttcaaacagtagctggcattttgcatttaagagttcgaccaaaagggacagaaatataaattgctaattattagcaatggaaatgaaaatgaatcagaagtgacaataaagatttctaaaattacaagtcgatgaaaaatgaatttactgtaaattggttaacacaaatcgctgtagtaacatatctgctatgtgaaatttttaaaataaaaaccagggcggagaaggggagaaggagatctctttccaaggcccaccacccaaattctaattcttagttcacagatccaaataataataatgtgttgttctatgacttctagtattaaaagtaaatataaatttactctgcatattaaacatttacattattctaggacttctcctttaaattacgatagtacacacacaaaaaccttatttatctaattgcctttcgttggcacataaagacaaagatcgtttgcaacataatcgcgttctgatacgtttgaccctctctagtcttcgtagaaggtggccaaggtttccgttaccccggcgtccctcgggcaccgcggaaacgaaactccatcaggctccgccccacggtctgcggagtgagccaatcagggcacagcctgcgttgaccgcgtgccgggtgtcatggcggcctgcaggtactgctgctcgtgcctccggctccggc

SEQ ID NO: 5 - DNA, 9 bp, Homo sapiens
  unsure 5: n = a, c, g, or t
gaaangaaa

SEQ ID NO: 6 - DNA, 9 bp, Homo sapiens
gaaacgaaa

SEQ ID NO: 7 - DNA, 9 bp, Homo sapiens
caactagaa

SEQ ID NO: 8 - RNA, 47 nt, Homo sapiens
uaauauuuauauauuuauauuuuuaaaauauuuauuuauuuauuuaa

SEQ ID NO: 9 - RNA, 11 nt, Homo sapiens
uauuuauuuaa

SEQ ID NO: 10 - RNA, 33 nt, Homo sapiens
uauuuauuuaaauauuuaaauuuuauauuuaau

SEQ ID NO: 11 - RNA, 34 nt, Homo sapiens
aauuaauuuauuauuuauuuauuauuuauuuauu

SEQ ID NO: 12 - RNA, 62 nt, Homo sapiens
guuuuuaauuuauuuauuaagauggauucucagauauuuauauuuuuuauuuuauuuuuuuu

SEQ ID NO: 13 - RNA, 111 nt, Homo sapiens
auuuacaugugccauuuuuuuaauucgaguaacccauauuuguuuaauuguauuuacauuauaaaucaagaaauauuuauuauuaaaaguaagucauuuauacaucuuaga

SEQ ID NO: 14 - DNA, 28 bp, Artificial Sequence
  Oligonucleotide primer
ggggtacccagtagctggcattttgcat

SEQ ID NO: 15 - DNA, 44 bp, Artificial Sequence
  Oligonucleotide primer
gaagatctggtcaacgcaggctgtgccctgattggctcactccg

SEQ ID NO: 16 - DNA, 43 bp, Artificial Sequence
  Oligonucleotide primer
cagaccgtggggcggagcctgatggagttctagttgcgcggtg

SEQ ID NO: 17 - DNA, 30 bp, Artificial Sequence
  Oligonucleotide primer
ggatcctctaagatgtataaatgacttact

SEQ ID NO: 18 - DNA, 24 bp, Artificial Sequence
  Oligonucleotide primer
cggaagtttagcattaatggattc

SEQ ID NO: 19 - DNA, 43 bp, Artificial Sequence
  Oligonucleotide primer
tgagtgcccgatcccgccgtggcagaaggaaaggaccatcgct

SEQ ID NO: 20 - DNA, 20 bp, Artificial Sequence
  Oligonucleotide primer
ttttgctcgttttgataatg

SEQ ID NO: 21 - DNA, 20 bp, Artificial Sequence
  Oligonucleotide primer
taatgggagaacctatttca

SEQ ID NO: 22 - DNA, 20 bp, Artificial Sequence
  Oligonucleotide primer
ctaattctcagtgatttttt

SEQ ID NO: 23 - DNA, 20 bp, Artificial Sequence
  Oligonucleotide primer
cgttgaccgcgtgccgggtg

SEQ ID NO: 24 - DNA, 20 bp, Artificial Sequence
  Oligonucleotide primer
attaaacaaatatgggttac

SEQ ID NO: 25 - DNA, 20 bp, Artificial Sequence
  Oligonucleotide primer
cggagtctcgctctgtcgcc

SEQ ID NO: 26 - DNA, 20 bp, Artificial Sequence
  Oligonucleotide primer
cggagccggaggcacgagca

SEQ ID NO: 27 - DNA, 20 bp, Artificial Sequence
  Oligonucleotide primer
caccgcggaaacgaaactcc

SEQ ID NO: 28 - DNA, 20 bp, Artificial Sequence
  Oligonucleotide primer
ggagtttcgtttccgcggtg

SEQ ID NO: 29 - DNA, 20 bp, Artificial Sequence
  Oligonucleotide primer
caccgcgcaactagaactcc

SEQ ID NO: 30 - DNA, 20 bp, Artificial Sequence
  Oligonucleotide primer
ggagttctagttgcgcggtg

SEQ ID NO: 31 - DNA, 19 bp, Artificial Sequence
  Oligonucleotide primer
atacggaggaacaactaaa

SEQ ID NO: 32 - DNA, 20 bp, Artificial Sequence
  Oligonucleotide primer
ggatcggcttctggaaattt

SEQ ID NO: 33 - DNA, 20 bp, Artificial Sequence
  Oligonucleotide primer
aaatttccagaagccgatcc

SEQ ID NO: 34 - DNA, 20 bp, Artificial Sequence
  Oligonucleotide primer
tgaagtcaaatcccgaccat

SEQ ID NO: 35 - DNA, 20 bp, Artificial Sequence
  Oligonucleotide primer
atggtcgggatttgacttca

SEQ ID NO: 36 - DNA, 20 bp, Artificial Sequence
  Oligonucleotide primer
caaatgtaacggtacaaagc

SEQ ID NO: 37 - DNA, 20 bp, Artificial Sequence
  Oligonucleotide primer
gctttgtaccgttacatttg

SEQ ID NO: 38 - DNA, 19 bp, Artificial Sequence
  Oligonucleotide primer
tgaaatttttatcttttat

SEQ ID NO: 39 - DNA, 24 bp, Artificial Sequence
  Oligonucleotide primer
taaaaatttcatgctgcactacga

SEQ ID NO: 40 - DNA, 21 bp, Artificial Sequence
  Oligonucleotide primer
tcattagttgcataaggagga

SEQ ID NO: 41 - DNA, 20 bp, Artificial Sequence
  Oligonucleotide primer
aaatagaagagaacttgggc

SEQ ID NO: 42 - DNA, 20 bp, Artificial Sequence
  Oligonucleotide primer
gatacaaagctttctcagca

SEQ ID NO: 43 - DNA, 20 bp, Artificial Sequence
  Oligonucleotide primer
tgctgagaaagctttgtatc

SEQ ID NO: 44 - DNA, 20 bp, Artificial Sequence
  Oligonucleotide primer
cattaatgctaaacttccgc

SEQ ID NO: 45 - DNA, 24 bp, Artificial Sequence
  Oligonucleotide primer
cggaagtttagcattaatggattc

SEQ ID NO: 46 - DNA, 22 bp, Artificial Sequence
  Oligonucleotide primer
gcaacagcagatgaaattggaa

SEQ ID NO: 47 - DNA, 19 bp, Artificial Sequence
  Oligonucleotide primer
atcctgagaagggtgaaat

SEQ ID NO: 48 - DNA, 20 bp, Artificial Sequence
  Oligonucleotide primer
tccatcacaatttttattgg

SEQ ID NO: 49 - DNA, 20 bp, Artificial Sequence
  Oligonucleotide primer
ccaataaaaattgtgatgga

SEQ ID NO: 50 - DNA, 20 bp, Artificial Sequence
  Oligonucleotide primer
aggttttgaaatagttttgt

SEQ ID NO: 51 - DNA, 20 bp, Artificial Sequence
  Oligonucleotide primer
acaaaactatttcaaaacct

SEQ ID NO: 52 - DNA, 20 bp, Artificial Sequence
  Oligonucleotide primer
aggttttgaaatagttttgt

SEQ ID NO: 53 - DNA, 20 bp, Artificial Sequence
  Oligonucleotide primer
acaaaactatttcaaaacct

SEQ ID NO: 54 - DNA, 20 bp, Artificial Sequence
  Oligonucleotide primer
ttttgataatggaacctgaa

SEQ ID NO: 55 - DNA, 20 bp, Artificial Sequence
  Oligonucleotide primer
gacctggtggctataactta

SEQ ID NO: 56 - DNA, 20 bp, Artificial Sequence
  Oligonucleotide primer
aagtctcttgcctcatgcat

SEQ ID NO: 57 - DNA, 20 bp, Artificial Sequence
  Oligonucleotide primer
atgcatgaggcaagagactt

SEQ ID NO: 58 - DNA, 20 bp, Artificial Sequence
  Oligonucleotide primer
gtatatactgctccaaattc

SEQ ID NO: 59 - DNA, 22 bp, Artificial Sequence
  Oligonucleotide primer
ataccgccacaataactgaaat

SEQ ID NO: 60 - DNA, 23 bp, Artificial Sequence
  Oligonucleotide primer
ataattttaccattacaccagta

SEQ ID NO: 61 - DNA, 23 bp, Artificial Sequence
  Oligonucleotide primer
cataacacacaacttgataacga

SEQ ID NO: 62 - DNA, 21 bp, Artificial Sequence
  Oligonucleotide primer
cctagggcagtaggatgttta

SEQ ID NO: 63 - DNA, 20 bp, Homo sapiens
taggcaacaggtaaggagtg

SEQ ID NO: 64 - DNA, 20 bp, Homo sapiens
acaggaaattgaaatatctt

SEQ ID NO: 65 - DNA, 20 bp, Homo sapiens
tacagtcagggtaaaaagtc

SEQ ID NO: 66 - DNA, 20 bp, Homo sapiens
tatgtttaagtcaggtgaca

SEQ ID NO: 67 - DNA, 20 bp, Homo sapiens
gcctttggtggtggtaagta

SEQ ID NO: 68 - DNA, 20 bp, Homo sapiens
gctgcttcaggttgactact

SEQ ID NO: 69 - DNA, 20 bp, Homo sapiens
cgaataataggtaagataat

SEQ ID NO: 70 - DNA, 20 bp, Homo sapiens
cttttggcagatcgttcaat

SEQ ID NO: 71 - DNA, 20 bp, Homo sapiens
tgatacacaggtagattctg

SEQ ID NO: 72 - DNA, 20 bp, Homo sapiens
cttttaccaggttctgtgta

SEQ ID NO: 73 - DNA, 20 bp, Homo sapiens
attaatggcggtaagtataa

SEQ ID NO: 74 - DNA, 20 bp, Homo sapiens
cttttttaagcttccgtagc

SEQ ID NO: 75 - DNA, 20 bp, Homo sapiens
ggacctgttggtaagttagg

SEQ ID NO: 76 - DNA, 20 bp, Homo sapiens
atacatttaggggcagtacg

SEQ ID NO: 77 - DNA, 20 bp, Homo sapiens
agtcagattggtaagttgtt

SEQ ID NO: 78 - DNA, 20 bp, Homo sapiens
tttaaatagtcatgttggaa

SEQ ID NO: 79 - DNA, 20 bp, Homo sapiens
atactcataagtaagaacat

SEQ ID NO: 80 - DNA, 20 bp, Homo sapiens
gcttaaatagttgctatgga

SEQ ID NO: 81 - DNA, 20 bp, Homo sapiens
gacaaagtttgtatacctga

SEQ ID NO: 82 - DNA, 20 bp, Homo sapiens
ttctttttagccagagatga

SEQ ID NO: 83 - DNA, 20 bp, Homo sapiens
caactaaaaggtaaatttta

SEQ ID NO: 84 - DNA, 20 bp, Homo sapiens
ttttctatagaaatttccag

SEQ ID NO: 85 - DNA, 20 bp, Homo sapiens
aatacaaaaggtaaacatac

SEQ ID NO: 86 - DNA, 20 bp, Homo sapiens
atcactataggtgcgatggt

SEQ ID NO: 87 - DNA, 20 bp, Homo sapiens
acaaacacaggtaatttata

SEQ ID NO: 88 - DNA, 20 bp, Homo sapiens
tcttttttaggtgctttgta

SEQ ID NO: 89 - DNA, 20 bp, Homo sapiens
cagctataaagtaagtaagt

SEQ ID NO: 90 - DNA, 20 bp, Homo sapiens
ttatttttagtgggataaaa

SEQ ID NO: 91 - DNA, 20 bp, Homo sapiens
gcactacgaggtaaggtgaa

SEQ ID NO: 92 - DNA, 20 bp, Homo sapiens
tttttttaagtttcctcctc

SEQ ID NO: 93 - DNA, 20 bp, Homo sapiens
cttgggcatggtaagtacag

SEQ ID NO: 94 - DNA, 20 bp, Homo sapiens
tctaatttaggtgctcttgc

SEQ ID NO: 95 - DNA, 20 bp, Homo sapiens
gagtcaaatggtattgaaat

SEQ ID NO: 96 - DNA, 20 bp, Homo sapiens
ctttctatagggtcatcttc

SEQ ID NO: 97 - DNA, 20 bp, Homo sapiens
atggattcaggtaaaagaat

SEQ ID NO: 98 - DNA, 20 bp, Homo sapiens
ccaattataggggttccaat

SEQ ID NO: 99 - DNA, 20 bp, Homo sapiens
agatattttggcaagtatat

SEQ ID NO: 100 - DNA, 20 bp, Homo sapiens
attcctgaagggaattgaag

SEQ ID NO: 101 - DNA, 20 bp, Homo sapiens
tgcattacaggtatttttta

SEQ ID NO: 102 - DNA, 20 bp, Homo sapiens
tccatattaggctgatatta

SEQ ID NO: 103 - DNA, 20 bp, Homo sapiens
caagcttcaggtgagctagt

SEQ ID NO: 104 - DNA, 20 bp, Homo sapiens
tctcttctagtggcaaaaaa

SEQ ID NO: 105 - DNA, 20 bp, Homo sapiens
tggacctgttgtaggtaata

SEQ ID NO: 106 - DNA, 20 bp, Homo sapiens
tacgttccagaaactgttca

SEQ ID NO: 107 - DNA, 20 bp, Homo sapiens
gctgaaacaggtaagagttg

SEQ ID NO: 108 - DNA, 20 bp, Homo sapiens
tttttaaaaggtgtaactat

SEQ ID NO: 109 - DNA, 20 bp, Homo sapiens
gcaaggatgatgtaagtata

SEQ ID NO: 110 - DNA, 20 bp, Homo sapiens
gcacttacagcaggagcagc

SEQ ID NO: 111 - DNA, 20 bp, Homo sapiens
ctgaaatcaggtaatatcaa

SEQ ID NO: 112 - DNA, 20 bp, Homo sapiens
atttttttgtagagatactg

SEQ ID NO: 113 - DNA, 20 bp, Homo sapiens
tcaacgaaaggtaaaaagac

SEQ ID NO: 114 - DNA, 20 bp, Homo sapiens
ttttttccagattaaacatc

SEQ ID NO: 115 - DNA, 20 bp, Homo sapiens
agaaattcaggtatttgtat

SEQ ID NO: 116 - DNA, 20 bp, Homo sapiens
tttaacacaggtgaaatact

SEQ ID NO: 117 - DNA, 10 bp, Homo sapiens
  unsure (5)...(6): n = a, c, g or t
gaaanngaaa

SEQ ID NO: 118 - DNA, 9 bp, Homo sapiens
  unsure (3)...(7): n = a, c, g or t
aannnnntt

SEQ ID NO: 119 - DNA, 24 bp, Artificial Sequence
  SYNTHETIC OLIGONUCLEOTIDE SENSE PRIMER FOR HEMAGGLUTININ TAG
gctagcatggcggcctgcaggtac

SEQ ID NO: 120 - DNA, 54 bp, Artificial Sequence
  SYNTHETIC OLIGONUCLEOTIDE ANTISENSE PRIMER FOR HEMAGGLUTININ TAG
ggatcctcaagcgtaatctggaacatcgtatgggtactgagaattagatgatga

(19) United States
(12) Patent Application Publication
(10) Pub. No.: US 2006/0140959 A1
(43) Pub. Date: Jun. 29, 2006

(54) Old-35, a gene associated with senescence and terminal cell differentiation, and uses thereof

(76) Inventors: Paul B. Fisher, Scarsdale, NY (US); Magdalena Leszczyniecka, Cambridge, MA (US); Dong-Chul Kang, Anyang (KR); Devanand Sarkar, Elmsford, NY (US)

Correspondence Address: BAKER & BOTTS, 30 ROCKEFELLER PLAZA, 44TH FLOOR, NEW YORK, NY, 10112, US

(21) Appl. No.: 11/186,718

(22) Filed: Jul. 21, 2005

Related U.S. Application Data
(63) Continuation of application No. PCT/US03/18382, filed on Jun. 9, 2003.
(60) Provisional application No. 60/442,105, filed on Jan. 23, 2003.
(60) Provisional application No. 60/466,040, filed on Apr. 28, 2003.
(60) Provisional application No. 60/466,678, filed on Apr. 29, 2003.

(51) Int. Cl. A61K 39/395; C12Q 1/68; C07H 21/04; C12P 21/06; C07K 14/82; C07K 16/30

(52) U.S. Cl. 424/155.1; 435/6; 435/69.1; 435/320.1; 435/325; 530/350; 530/388.8; 536/23.5

(57) ABSTRACT

The present invention relates to the old-35 gene, its encoded protein, and its promoter sequence. The old-35 gene is associated with terminal differentiation and senescence of cells, and as such the gene and its related molecules may be used in the control of cell proliferation and in the modulation

### CROSS-REFERENCE TO RELATED APPLICATION(S)

This application claims priority from U.S. Application Ser. Nos. 60/442,105 filed Jan. 23, 2003; 60/466,040 filed Apr. 28, 2003 and 60/466,678 filed Apr. 29, 2003, the entire disclosures of which are incorporated herein by reference.

### GRANT SUPPORT

The subject matter of this provisional application was supported in part by National Institutes of Health Grants 5R01CA035675 and 5R01CA074468, and National Cancer Institute Grant CA97318, so that the United States government has certain rights herein.

### 1. INTRODUCTION

The present invention relates to the old-35 gene, its encoded protein, and its promoter sequence. The old-35 gene is associated with terminal differentiation and senescence of cells, and as such the gene and its related molecules may be used in the control of cell proliferation and in the modulation of differentiation.

### 2. BACKGROUND OF THE INVENTION

Current cancer therapies are highly toxic and often nonspecific. A potentially less toxic approach to treating this prevalent disease employs agents that modify cancer cell differentiation, termed “differentiation therapy” (Leszczyniecka et al., 2001, Pharmacol. Ther. 90(2-3): 105-156). This approach is based on the tacit assumption that many neoplastic cell types exhibit reversible defects in differentiation, which upon appropriate treatment, result in tumor reprogramming and a concomitant loss in proliferative capacity and induction of terminal differentiation or apoptosis (programmed cell death). Laboratory studies that focus on elucidating mechanisms of action are demonstrating the effectiveness of differentiation therapy, which is now beginning to show translational promise in the clinical setting.

In searching for agents useful for differentiation therapy, researchers have studied model systems which reduce or eliminate the malignant characteristics of cancer cells. In one such model system, the HO-1 line of human melanoma cells, when treated with interferon β (“IFN-β”) and the antileukemic compound mezerein, manifests growth arrest, altered cellular morphology, modifications in antigenic phenotype and an increase in melanogenesis, all indices of a more “differentiated” cellular phenotype (Graham et al., 1991, Cancer Immunol. Immunother 32: 382-390; Jiang et al., 1994, Mol. Cell. Diff. 2: 221-239). Using this system, a number of genes associated with differentiation have been identified, including mda-7 (U.S. Pat. Nos. 6,355,622, 5,710,137 and 5,643,761, by Fisher et al.). Mda-7 is currently in clinical studies as a gene therapy anti-cancer agent; results of a phase 1 study by Introgen Therapeutics demonstrate that up to 70 percent of tumor cells died via apoptosis after tumors were injected with a single dose of INGN 241, a modified adenoviral vector that carries the mda-7 gene (Ad.mda-7) (presented at the June 2002 Annual Meeting of the American Society of Gene Therapy).

As the ultimate goal of cancer therapy is to kill malignant cells, another avenue of research studies the process by which normal cells reach the end of their life span and die (Huang et al., 2002, Cancer Res. 62(11):3226-3232; Schmitt et al., 2002, cell 109(3):335-346). The object of such research is to identify genes that effect senescence in normal cells, to be used to promote cell death in cancer cells.

International Patent Application No. PCT/US00/02920, published Aug. 10, 2000 as Publication No. WO 00/46391, inventors Fisher and Leszczyniecka, incorporated by reference in its entirety herein, discloses the discovery of a gene, associated with terminal differentiation and senescence, termed old-35. The gene was discovered as follows. On the theory that specific differentially expressed genes may be present within a terminally differentiated cDNA library that also display modified expression during cellular senescence, a temporally spaced subtracted differentiation inducer-treated HO-1 melanoma library was screened with a probe constructed from senescent human fibroblast total RNA. This experiment yielded twenty-eight known and ten novel cDNAs. Subsequent Northern and reverse Northern blotting analyses revealed differential expression of the identified cDNAs. Expression of one of these cDNAs, old-35, was found to be interferon-inducible and restricted to terminal differentiation and senescence. Old-35 was found to exhibit high homology to a 3′-5′ RNA exonuclease, polyribonucleotide phosphorylase (PNPase), an important enzyme implicated in the degradation of bacterial messenger RNAs (Portier et al., 1981, Mol. Gen. Genet. 183: 298-305). Use of old-35 and nucleic acids that specifically hybridize thereto in various methods were disclosed, including methods for inhibiting the growth of cancer cells and for determining whether a cell is senescent, growth arrested, and/or terminally differentiated.

### 3. SUMMARY OF THE INVENTION

The present invention is based, at least in part, on the further characterization of the old-35 gene and its promoter and on the discovery of elements within the old-35 promoter which confer interferon inducibility. It has been determined that the nucleic acid sequence of the old-35 gene reported in International Patent Application No. PCT/US00/02920 is the sequence of a variant of the wild type form of old-35. Relative to the variant, the wild-type nucleic acid sequence contains an additional cytosine residue between residues 2089 and 2090 of SEQ ID NO:39 of PCT/US00/02920. This single base insertion, residue 2159 of the nucleic acid sequence set forth in FIG. 1A and SEQ ID NO:1 herein, alters the open reading frame such that the wild type protein, as set forth in FIG. 1A and SEQ ID NO:2, contains an additional 61 amino acids relative to the OLD-35 protein previously disclosed (a total of 766 amino acids compared with 705 in the variant protein). It should be noted that amino acids 1-696 of the wild-type and variant proteins are the same.

In addition to the correct nucleic acid and amino acid sequences of wild-type old-35 gene and its encoded protein, the present invention provides for methods for using such molecules in the modulation of cell differentiation and proliferation.

In other embodiments, the present invention provides for nucleic acid molecules comprising the old-35 promoter and variants thereof. The old-35 promoter and its variants may be used in assays to identify agents that increase activity of the promoter and could be used to promote terminal differentiation and to suppress proliferation of cells, for example in the treatment of cancer. In addition, as the old-35 promoter is induced by interferon and variants lacking this inducibility have been identified, assays comparing the effects of a test agent on the activity of old-35 promoter constructs either containing or lacking interferon-inducible elements may be used to identify agents which activate the promoter by either augmenting interferon inducibility or by a mechanism complementary to interferon induction. Further, the old-35 promoter may be used in gene therapy applications to introduce genes that would be selectively expressed in the context of cellular senescence, terminal differentiation, or interferon therapy.

### 4. BRIEF DESCRIPTION OF THE FIGURES

FIG. 1A-C. (A) Sequence of old-35 cDNA (SEQ ID NO:1) and the OLD-35 protein (SEQ ID NO:2). * indicates an in-frame stop codon. (B) Representation of two alternate forms of old-35 detected on Northern blots of HO-1 cells treated with IFN-β (2000U/ml, 18 h). Dark gray region represents 5′ UTR, black region represents protein coding region, light gray represents 3′ UTR common to both old-35 variants, white region represents 3′ UTR present only in the longer form of old-35. The region between the arrows indicates the EST that was isolated from the OPS library screen and the arrows indicate the directions in which the rest of the gene was cloned. (C) Northern analysis of IFN-β treated HO-1 (2000U/ml, 18 h) human melanoma cells to determine sizes of the old-35 variants. Two membranes containing the same RNAs were probed with either the coding region (black) left panel or with the 3′ UTR of the longer variant (white) right panel. The smearing seen on the right is due to high homology of AU rich elements found in the 3′ UTR of the old-35 gene to many other mRNAs expressed in human cells.

FIG. 2A-F. (A) Northern blotting analysis of old-35 expression in HO-1 cells either untreated (control) or treated for 18 h with IFN-β (2000 units/ml), MEZ (10 ng/ml) or IFN-β+MEZ (2000 units/ml+10 ng/ml) (B) Time course treatment of HO-1 cells with IFN-β (2000 units/ml). (C) IFN-β dose response in HO-1 cells. Cells were untreated (U) or treated with 0.1 units/ml to 2000 units/ml for 7 h. (D) IFN-β dose response in HO-1 cells. Cells were untreated (U) or treated with 1 unit/ml to 2000 units/ml for 24 h. (E) Response of HO-1 cells to various types of IFNs and TNF-α. Iα (IFN-α, 1000U/ml), Iβ (IFN-β, 1000U/ml), Iγ (IFN-γ, 1000 U/ml), TNF-α (10 ng/ml) treatment for 18 h. (F) Effect of IFN-β and Poly IC on old-35 and gapdh expression in HO-1 cells. U (Untreated) or treated for 24 h with IFN-β (2000 units/ml), Poly IC, 1× (10 μg/ml) or 2× (20 μg/ml).

FIG. 3A-C. (A) Expression of old-35 and gapdh in various normal and cancer cell lines and following 18 h treatment with IFN-β. Cells were either untreated or treated with IFN-β (2000 units/ml). Lane designation: 1, WM35; 2, FO-1; 3 MeWo; 4, WM238; 5, C8161; 6, C8161-6.3; 7, HO-1; 8, HO-1+IFN-β; 9, HeLa; 10, HeLa+IFN-β; 11, human skin fibroblasts; 12, human skin fibroblasts+IFN-β. (B) Analysis of old-35 and gapdh expression in various human melanomas without (−) or with (+) IFN-β (2000 units/ml) treatment for 18 h. (C) Analysis of old-35 and gapdh expression in human breast carcinoma (MDA-MB-157, MDA-MB-231 and MCF-7) or human osteosarcoma (Saos2) cells without (−) or with (+) IFN-β (2000 units/ml) treatment for 18 h.

FIG. 4A-B. Primer extension analysis of the old-35 gene. (A) Identification of the initiation start site by primer extension in HO-1 cells treated with IFN-β with an antisense primer (PE) (see FIG. 11). A sequencing reaction with PE primer was run in parallel for size estimation. C, T, A, and G stand for cytosine, thymidine, adenine, and guanidine, respectively. P stands for the primer extension reaction. (B) Graphical representation of the cloned 2-kb old-35 promoter. The immediate 400-bp sequence of the old-35 promoter (SEQ ID NO:3) including the transcription initiation site is shown (the arrow). Additional sites that may be related to IFN signaling or to the constitutive activity of the old-35 promoter are underlined and bolded. The numbers below the elements indicate the initial bp at which the element is located.

FIG. 5A-D. (A) Graphical representation of various deletions in the old-35 promoter. The construct sizes are not drawn to scale. Numbers on the right describe the approximate construct sizes. (B) Luciferase assays with various old-35 promoter constructs monitor de novo activity and the effect of various deletions on this activity in HO-1 cells. The relative activity represents the luciferase reading per 20 μg of protein. The error bars represent the S.D. for three independent experiments containing three replicate samples for each assay. (C) Luciferase assays in HO-1, FO-1, HeLa, and 2fTGH cells with various old-35 promoter constructs. Data presented as fold induction of luciferase expression upon IFN-β treatment vs. expression in untreated HO-1. To calculate fold induction of the old-35 promoter by IFN-β, luciferase readings in the IFN-β treated HO-1 samples were divided by luciferase activity in the untreated HO-1 controls. The values presented represent the average of three independent experiments±S.E. using triplicate samples for each experiment. Abbreviations: ISRE (Interferon Stimulated Response Element), GAS (Gamma Activation Site), IRF-1 (Interferon Response Factor 1). (D) Nucleic acid sequence of old-35 promoter (SEQ ID NO:4). The end of the promoter and the beginning of the gene is indicated by underlining of the gene sequence. The portion of the promoter incorporated in p2000 is set forth in boldface print.

FIG. 6A-D. (A) Sequences of the consensus ISRE (SEQ ID NO: 5), the old-35 ISRE (SEQ ID NO:6) and the mutant old-35 ISRE (SEQ ID NO:7) used for gel shift (EMSA) assays. (B) Luciferase assay with a p400 containing wild type ISRE and a p400/mISRE containing a mutant ISRE in HO-1 and HeLa cells. Fold induction of the old-35 promoter by IFN-β was calculated as described in the legend to FIG. 5A-D. The values presented represent the average of three independent experiments±S.E. using triplicate samples per assay. (C) Gel shift assay with wild type (WT) and mutant (MT) ISRE oligonucleotides in HeLa cells. (D) Gel shift assay with wild type (WT) and mutant (MT) ISRE oligonucleotides in untreated and IFN-β treated HO-1 cells. The arrow points to the ISGF3 complex. Abbreviations: cWT (cold wild type), cMT (cold mutant), WT ([γ-³²P]ATP labeled wild type), and MT ([γ-³²P]ATP labeled mutant).

FIG. 7A-B. (A) Northern blot analysis of old-35 expression in 2fTGH, U1, U3, U4, and U5 cell lines treated with IFN-β for 7 h. Ethidium bromide (EtBr) staining was used as a loading control. (B) Luciferase assays of p1000 in 2fTGH, U1, U3, U4, and U5 cell lines upon IFN-β (2000U/ml) treatment for 7 h.

FIG. 8A-E. (A) Schematic representation of the 3′ UTRs (Untranslated Regions) of M-CSF (SEQ ID NO:8), IFN-α (SEQ ID NO:9), IL-2 (SEQ ID NO:10), TNF-α (SEQ ID NO:11), c-fos (SEQ ID NO:12) and old-35 (SEQ ID NO:13). AUUUA repetitive sequences found in the 3′ UTRs of these genes, underlined, have been implicated in controlling mRNA stability of these transcripts. (B) Half-life of old-35 mRNA in untreated and IFN-β (2000 units/ml) treated HO-1 cells determined by Actinomycin D (AD) treatment. For mRNA half-life studies, HO-1 cells were first untreated or treated with IFN-β (2000 units/ml) and then cultured for the indicated times in medium lacking or containing actinomycin D (AD) (5 μg/ml). Samples were withdrawn every 2 h and total RNAs were extracted as in materials and methods. (C) De novo activity of p400 and p400/UTR in HO-1 cells. The relative activity represents the luciferase reading per 20 μg of protein. The error bars represent the S.D. of three independent experiments containing triplicate samples per experimental point. (D) Luciferase assays with p400 and p400/UTR showing fold induction of luciferase by IFN-β (2000 units/ml). Fold upregulation of old-35 promoter activity by IFN-β was determined as described in the legend to FIG. 5A-D. The values presented represent the average of three independent experiments±S.E. using triplicate samples per experimental point. (E) Cycloheximide (“CHX”; 50 μg/ml) treatment of HO-1 cells with and without treatment with IFN-β (2000 units/ml). HO-1 cells were either treated with CHX alone, IFN-β alone or a combination of agents. For CHX treatment, HO-1 cells were pre-treated with CHX for 3 h and treated with IFN-β (2000 units/ml) for the indicated times. For controls, HO-1 cells were treated with either IFN-β (2000 units/ml) or CHX (50 μg/ml). Abbreviations: M-CSF (Macrophage Colony Stimulating Factor), IFN-α (Human Interferon α), IL-2 (Interleukin 2), TNF-α (Human Tumor Necrosis Factor alpha), U-untreated, AD (Actinomycin D treated), CHX (Cycloheximide treated).

FIG. 9A-D. Chromosomal localization of the old-35 gene and the old-35 pseudogenes. (A) Total human chromosomes. (B) Close-up of chromosome 2p. (C) Tabulation of the results shown in (A) and (B). (D) Chromosomal localization of old-35 obtained using the Radiation Hybrid Panel Gene Bridge 4 (GB4). Results were obtained from NCBI database using a BLAST search with the old-35 full-length cDNA sequence.

FIG. 10. Exon/intron structure of the old-35 gene and graphical representation of the old-35 gene. The numbers above the picture represent the exon numbers. The small numbers below the exon numbers indicate the intron sizes. The numbers below the figure indicate the exon sizes. * Since there are two mRNA variants, the last exon in the 4.3 kbp old-35 variant is 1960-bp.

FIG. 11. Primer list (SEQ ID NOS:14-62).

FIG. 12. Exon/intron organization of Old-35 gene (SEQ ID NOS: 63-116).

FIG. 13A-F. Transduction by Ad.old-35 induces death and morphological changes in HO-1 melanoma cells. (A) Schematic representation of the Ad.old-35 vector (B) Untransduced HO-1 cells after five days of culture. (C) HO-1 cells transduced by Ad.vec at a MOI of 100 pfu/cell and cultured for five days. (D) HO-1 cells transduced by Ad.old-35 at a MOI of 100 pfu/cell and cultured for five days. (E) Untransduced HO-1 cells after 10 days of culture. (F) Untransduced HO-1 cells after five days of culture in the presence of IFN-β (2000 units/ml) and mezerein (10 ng/ml). The cells were photographed using a light microscope at 100×.

FIG. 14A-F. Transduction by Ad.old-35 inhibits growth of melanoma cells, normal melanocytes and normal melanocytes immortalized with SV40 Tag. (A) HO-1 cells. (B) FO-1 cells. (C) WM278 cells. (D) WM35 cells. (E) MeWo cells. (F) FM-516. Cells were transduced by either Ad.vec or Ad.old-35 at a MOI of 100 pfu/cell and cell viability was monitored by standard MTT assay.

FIG. 15A-B. Transduction by Ad.old-35 reduces colony formation in HO-1 cells. (A) Plating efficiency in HO-1 cells transduced by either Ad.vec or Ad.old-35 at a MOI of 100 pfu/cell. (B) Photomicrograph of colony formation in HO-1 cells transduced by either Ad.vec or Ad.old-35.

FIG. 16A-B. Transduction by Ad.old-35 induces apoptosis and inhibits DNA synthesis in HO-1 cells. (A) HO-1 cells were either non-transduced (top panel) or transduced with Ad.vec or Ad.old-35 at a MOI of 100 pfu/cell. Cell cycle was then analyzed by flow cytometry at four days post-transduction. M1 represents the apoptotic cell population. (B) Proportion of cells at G0/G1 (top), S (middle), or G2/M (lower) following transduction by Ad.vec (left bars) or Ad.old-35 (right bars).

FIG. 17. Transduction of HO-1 cells by Ad.old-35 reduces telomerase activity, as measured using TeloTAGGG Telomerase PCR ELISA^{PLUS} (Roche).

FIG. 18. Transduction of HO-1 cells by Ad.old-35 downregulates c-myc and bcl-xl expression and upregulates mad-1 expression.

FIG. 19A-B. Overexpression of c-myc protects the HO-1 cells from Ad.old-35-mediated killing. (A) Photomicrograph of colony formation in HO-1 cells transfected by the control vector or by the c-myc-expressing plasmid, and transduced by either Ad.vec or Ad.old-35. (B) Plating efficiency in HO-1 cells transfected by the control vector or by the c-myc-expressing plasmid, and transduced by either Ad.vec or Ad.old-35, at the MOIs indicated.

FIG. 20. Overexpression of bcl-xl protects the HO-1 cells from Ad.old-35-mediated killing.

FIG. 21. Transduction of HOI-1 cells by Ad.old-35 downregulates p21 and upregulates p27.

FIG. 22. Transduction of HOI-1 cells by Ad.old-35 induces phosphorylation of double-stranded RNA dependent protein kinase (PKR) and eIF2α.

FIG. 23. Transduction of HOI-1 cells by Ad.old-35 induces the expression of GADD153 and fibronectin.

FIG. 24A-C. Transduction of HOI-1 cells by Ad.old-35 induces senescence associated β-galactosidase activity (SA-β-GAL). (A) Non-transduced HO-1 cells. (B) HO-1 cells transduced by Ad.vec. (C) HO-1 cells transduced by Ad.old-35. The cells were photographed using a light microscope at 100×.

FIG. 25A-D. Infection with Ad.hPNPase^{old-35} induces G1 arrest, decreases S-phase and induces apoptosis in HO-1 human melanoma cells. A. HO-1 cells were either uninfected or infected with Ad.vec or Ad.hPNPase^{old-35} at an m.o.i. of 100 pfu/cell and cell cycle was monitored by flow cytometry 4 days post-infection. B. Percentage of cell population in different phases of the cell cycle following the infection protocol as in A. White, gray and black bars represent control, Ad.vec-infected and Ad.hPNPase^{old-35}-infected cells, respectively. C. Cell cycle analysis by flow cytometry of HO-1 cells either uninfected or infected with Ad.vec or Ad.hPNPase^{old-35} at an m.o.i. of 25 pfu/cell 3 days post-infection. D. Percentage of cell population in different phases of the cell cycle following the infection protocol as in B at 3, 6 and 8 days post-infection. White, gray and black bars represent control, Ad.vec-infected and Ad.hPNPase^{old-35}-infected cells, respectively.

FIG. 26A-B. Infection with Ad.hPNPase^{old-35}-inhibits ³H-thymidine incorporation and telomerase activity. A. HO-1 cells were either uninfected or infected with Ad.vec (m.o.i. 100 pfu/cell) or Ad.hPNPase^{old-35} at an m.o.i. of either 25 or 50 pfu/cell. ³H thymidine incorporation assay was performed 4 days post-infection. The data represent mean±SD of two independent experiments each performed in triplicates. B. HO-1 cells were either untreated (filled diamonds) or treated with IFN-beta+MEZ (filled squares) for the indicated time periods and telomerase activity was measured as in Materials & Methods. The data represents mean±SD of two independent experiments.

FIG. 27A-C. Effect of Ad.hPNPase^{old-35} on Myc, Mad1, Max and EF1 alpha expression and cell growth. A. HO-1 cells were either uninfected or infected with either Ad.vec or Ad.hPNPase^{old-35} at an m.o.i. of 100 pfu/cell. Samples were collected on day 4 post-infection in case of control and Ad.vec-infected cells. Expression of the mRNAs indicated was analyzed by Northern blot analysis. B. HO-1 cells were treated as in A. Expression of the proteins indicated was analyzed by Western blot analysis. C. HEK-293 cells were transfected with either empty vector or p290-myc. The cells were harvested after 48 h and the expression of Myc was analyzed by Western blot analysis.

FIG. 28A-C. hPNPaseold-35 degrades c-myc mRNA in vitro. A. HEK-293 cells were transfected with either empty vector or hPNPaseold-35-HA. The cells were harvested after 48 h and the expression of hPNPaseOLD-35-HA was analyzed by Western blot analysis using anti-HA antibody. B. HO-1 cells were transfected with either empty vector or hPNPaseold-35-HA. Colony formation assays were performed as described in Materials & Methods. The columns are graphical representations of the plating efficiency. The data represents mean±SD of two independent experiments each done in pentaplicate. C. In vitro degradation assay was performed as described in Materials & Methods. The expression of c-myc, GAPDH, GADD34 and c-jun mRNAs were detected by Northern blot analysis.

FIG. 29. Expression profiles of regulators of G1 checkpoint following Ad.hPNPase^{old-35} infection. Infection protocol was performed as described in FIG. 27A. Expression of the indicated proteins were detected by Western blot analysis.

FIG. 30A-D. Antisense inhibition of hPNPase^{old-35} provides protection against IFN-beta-mediated growth inhibition. A. HO-1 cells were transfected with 5 micrograms each of either pcDNA3.1 or hPNPase^{old-35}-HA. The cells were infected with either Ad.vec or Ad.hPNPase^{old-35}AS at an m.o.i. of 50 or 100 pfu/cell 6 h later. The expression of hPNPase^{OLD-35}-HA was analyzed in cell lysates by Western blot analysis using Anti-HA antibody 48 h post-infection. B. HO-1-pREP4 and HO-1-hPNPase^{old-35}AS cells were infected with either Ad.vec or Ad.hPNPase^{old-35}AS at an m.o.i. of 5, 25 and 100 pfu/cell. 24 h later, the cells were treated with IFN-beta for 5 days and cell viability was assayed by MTT assay. C. HO-1-pREP4 and HO-1-hPNPase^{old-35}AS cells were infected as in 30B and were treated with MEZ (10 ng/ml). Cell viability was assayed after 5 days. D. HO-1-pREP4 and HO-1-hPNPaseold-35AS cells were infected as in 30B and were treated with IFN-beta+MEZ. Cell viability was assayed after 5 days. For B, C and D the column numbers represent the legends at the bottom of the figure and the data represent mean+SD of two independent experiments each performed in octaplicates.

FIG. 31A-B. Effect of Ad.hPNPase^{old-35} on the expression of pro- and anti-apoptotic molecules. A. Infection protocol was performed as described in FIG. 27A. Expressions of the indicated proteins were detected by Western blot analysis. B. HO-1 (white column), HO-1-bcl-2 (gray column) and HO-1-bcl-xL (black column) cells were either uninfected or infected with Ad.vec or Ad.hPNPase^{old-35} at an m.o.i. of 100 pfu/cell. Cell viability was determined by MTT assay 5 days post-infection. The data represent mean+SD of three independent experiments each performed in octaplicates.

### 5. DETAILED DESCRIPTION OF THE INVENTION

For clarity of presentation, and not by way of limitation, the detailed description of the invention is divided into the following subsections:
(i) old-35 nucleic acid molecules and proteins;
(ii) uses of old-35;
(iii) the old-35 promoter and its variants; and
(iv) uses of the old-35 promoter and its variants.

### 5.1 Old-35 Nucleic Acid Molecules and Proteins

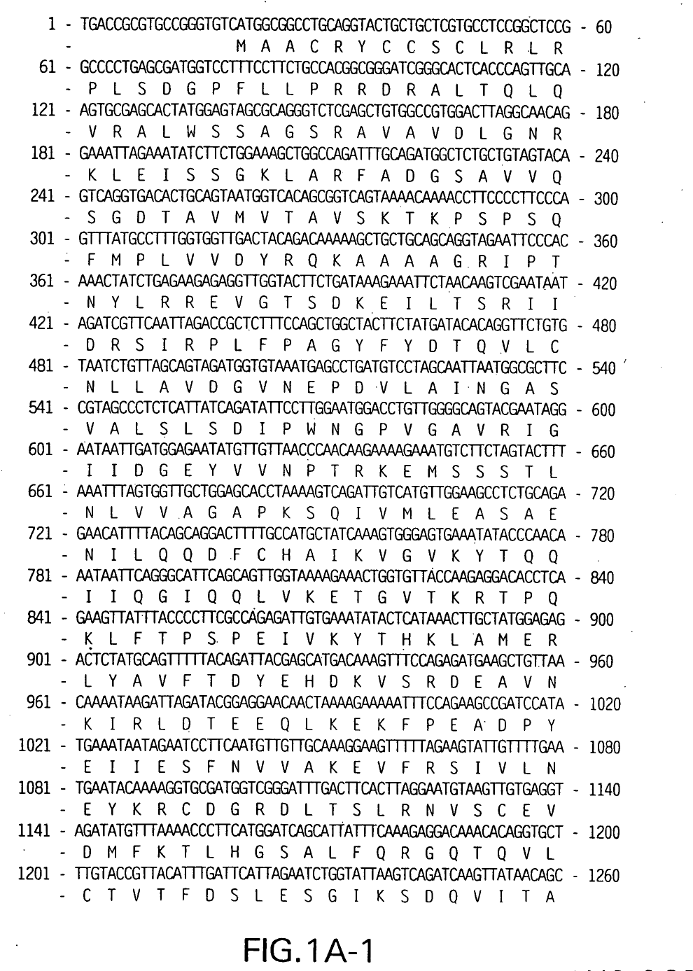

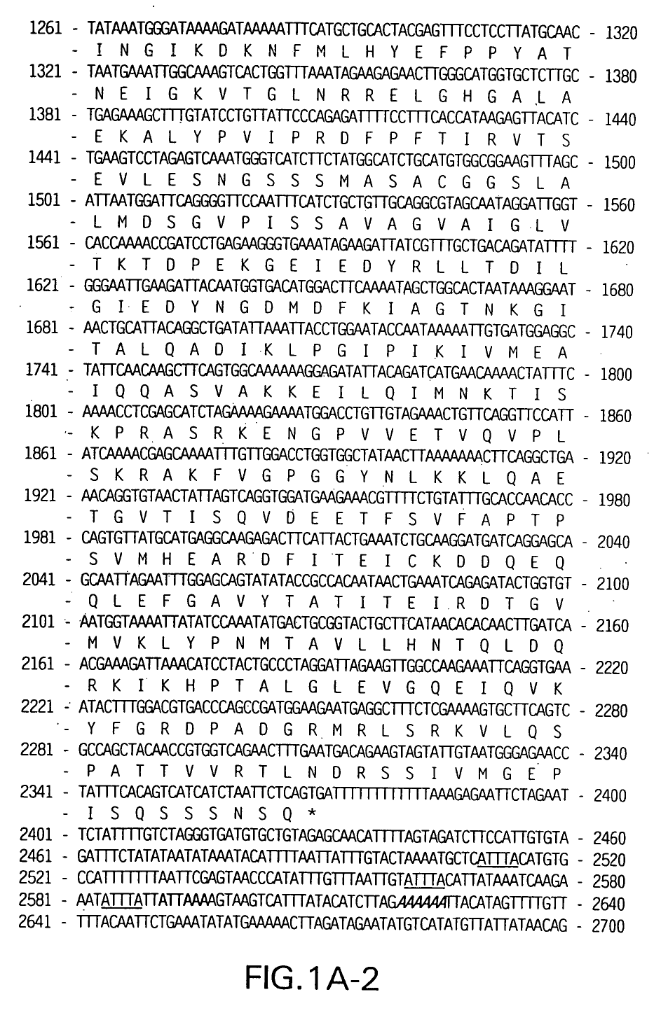

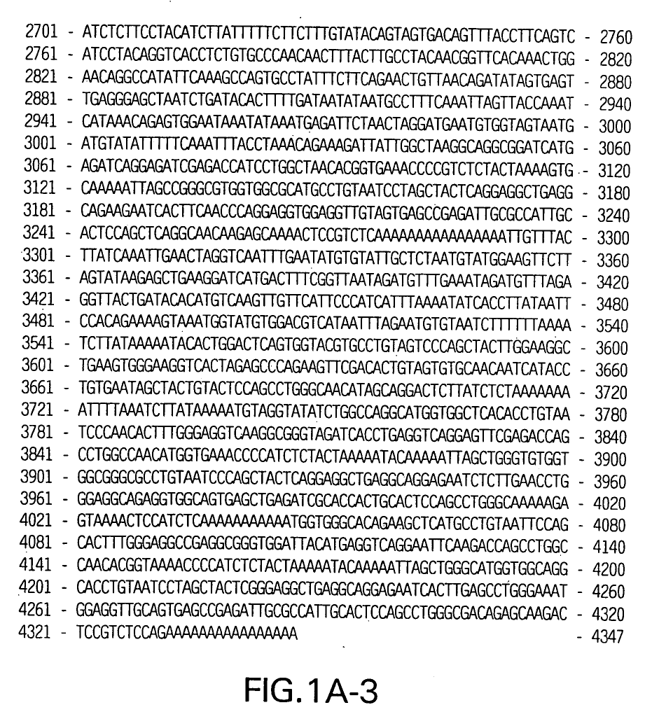

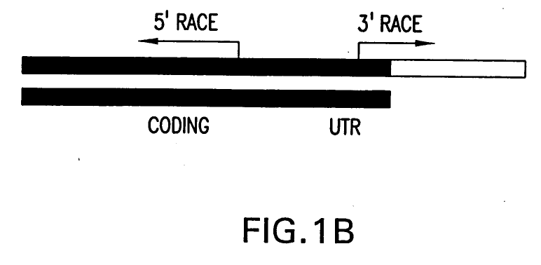

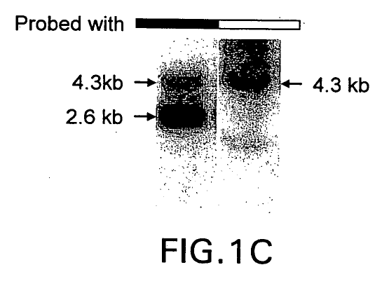

The present invention provides for isolated nucleic acid molecules encoding a protein having a sequence as set forth in SEQ ID NO:2 (FIG. 1A). Such molecules are referred to herein as “old-35 nucleic acids.” In one specific, non-limiting embodiment, the nucleic acid molecule has a sequence as set forth in SEQ ID NO:1 (FIG. 1A). An old-35 nucleic acid may be comprised in a larger nucleic acid as set forth below; in addition to being linked to expression control elements, the old-35 nucleic acid may be comprised in a nucleic acid encoding a larger protein, for example an OLD-35 fusion protein.

As stated in the preceding paragraph, an old-35 nucleic acid may be linked to one or more element associated with gene expression. Such elements may include one or more of a promoter/enhancer element, a transcription start site, a transcription termination signal, a polyadenylation site, a ribosome binding site, etc.

An old-35 nucleic acid may be operatively linked to a suitable promoter element, which may be its endogenous promoter or a variant thereof (as described in section 5.3 below) or a heterologous promoter. Examples of suitable heterologous promoters include but are not limited to the cytomegalovirus immediate early promoter, the Rous sarcoma virus long terminal repeat promoter, the human elongation factor 1α promoter, the human ubiquitin c promoter, etc. It may be desirable, in certain embodiments of the invention, to use an inducible promoter. Non-limiting examples of inducible promoters include the murine mammary tumor virus promoter (inducible with dexamethasone); commercially available tetracycline-responsive or ecdysone-inducible promoters, etc. In specific non-limiting embodiments of the invention, the promoter may be selectively active in cancer cells; one example of such a promoter is the PEG-3 promoter, as described in International Patent Application No. PCT/US99/07199, Publication No. WO 99/49898 (published in English on Oct. 7, 1999); other non-limiting examples include the prostate specific antigen gene promoter (O'Keefe et al., 2000, Prostate 45:149-157), the kallikrein 2 gene promoter (Xie et al., 2001, Human Gene Ther. 12:549-561), the human alpha-fetoprotein gene promoter (Ido et al., 1995, Cancer Res. 55:3105-3109), the c-erbB-2 gene promoter (Takakuwa et al., 1997, Jpn. J. Cancer Res. 88:166-175), the human carcinoembryonic antigen gene promoter (Lan et al., 1996, Gastroenterol. 111:1241-1251), the gastrin-releasing peptide gene promoter (Inase et al., 2000, Int. J. Cancer 85:716-719), the human telomerase reverse transcriptase gene promoter (Pan and Koenman, 1999, Med. Hypotheses 53:130-135), the hexokinase II gene promoter (Katabi et al., 1999, Human Gene Ther. 10:155-164), the L-plastin gene promoter (Peng et al., 2001, Cancer Res. 61:4405-4413), the neuron-specific enolase gene promoter (Tanaka et al., 2001, Anticancer Res. 21:291-294), the midkine gene promoter (Adachi et al., 2000, Cancer Res. 60:4305-4310), the human mucin gene MUC1 promoter (Stackhouse et al., 1999, Cancer Gene Ther. 6:209-219), and the human mucin gene MUC4 promoter (Genbank Accession No. AF241535).

An old-35 nucleic acid may be incorporated into a vector for replication and/or expression. Suitable vectors include but are not limited to plasmids, phage, phagemids, and viruses, as are known in the art.

Where the vector is an expression vector, suitable expression vectors include virus-based vectors and non-virus based DNA or RNA delivery systems. Examples of appropriate virus-based gene transfer vectors include, but are not limited to, those derived from retroviruses, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6(2):113-138; Connelly, 1999, Curr. Opin. Mol. Ther. 1(5):565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy.

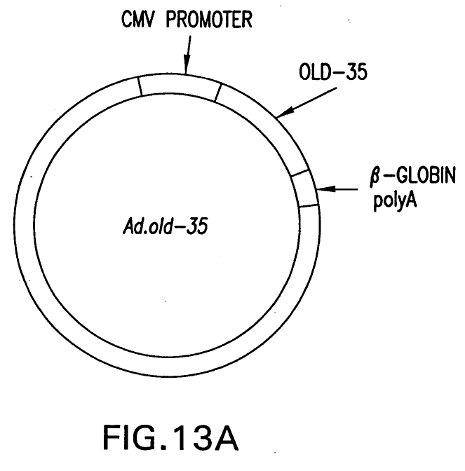

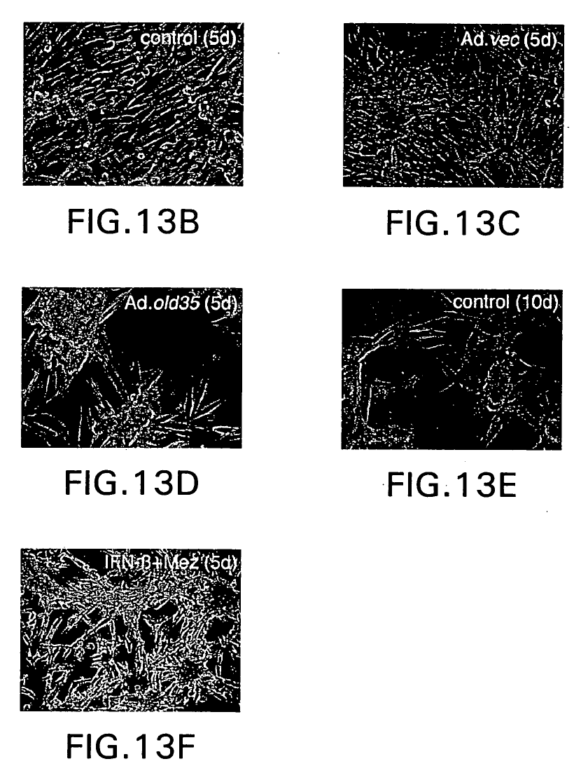

In a preferred embodiment, the vector is a recombinant adenovirus vector that comprises a cassette for the expression of the OLD-35 protein in various target cells. A schematic diagram of one possible recombinant adenovirus vector, Ad.old-35, is shown in FIG. 13A. In this vector, transcription of the old-35 cDNA is driven by the cytomegalovirus immediate early (CMV) promoter. One of ordinary skill in the art would recognize that the CMV promoter may be replaced by other suitable promoter element, such as those described above, to achieve constitutive, inducible, or cell- or tissue-specific promoters.

Non-limiting examples of non-virus-based delivery systems which may be used according to the invention include, but are not limited to, so-called naked nucleic acids (Wolff et al., 1990, Science 247:1465-1468), nucleic acids encapsulated in liposomes (Nicolau et al., 1987, Methods in Enzymology 149:157-176), nucleic acid/lipid complexes (Legendre and Szoka, 1992, Pharmaceutical Research 9:1235-1242), and nucleic acid/protein complexes (Wu and Wu, 1991, Biother. 3:87-95).

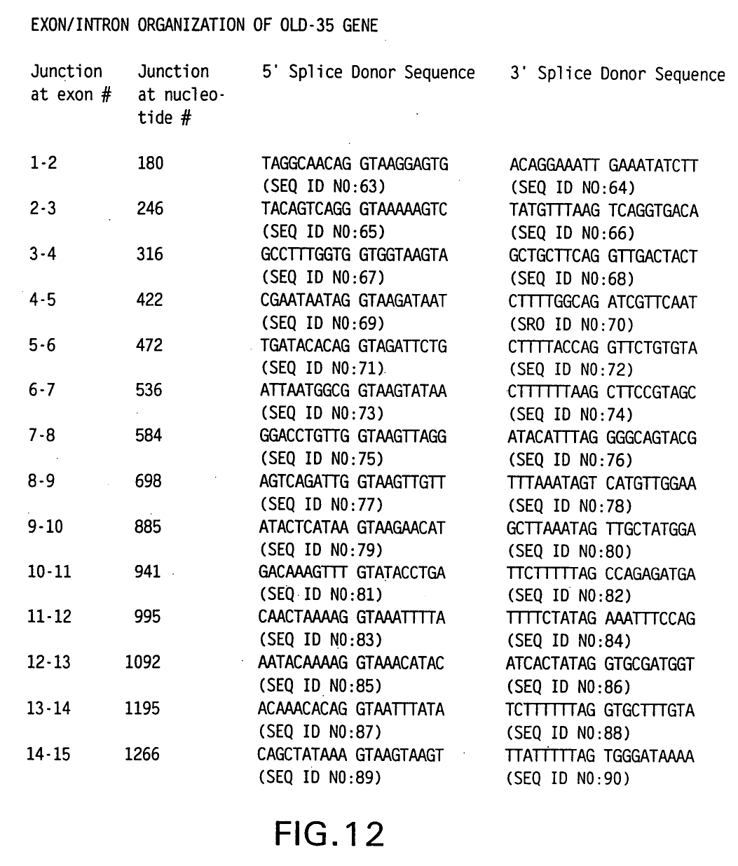

The present invention further provides for nucleic acid fragments of the old-35 gene, for example intronic and exonic sequences and combinations thereof. Primers which may be used to produce such fragments are also provided for, as are set forth in FIG. 12 and SEQ ID NOS: 63-116.

The present invention further provides for an OLD-35 protein having a sequence set forth as SEQ ID NO:2 (FIG. 1A). This protein may optionally be comprised in a larger protein, for example, as a fusion protein. The protein may also be linked to one or more element, for example, a carbohydrate, detectable label (for example a radioactive, fluorescent, cofactor, or antibody label), polyalkylene oxide (for example polyethylene glycol (“PEG”)) or another chemical crosslinker, a nucleic acid, a lipid, etc.

### 5.2 Uses of Old-35 Nucleic Acids and Porteins

An old-35 nucleic acid may be used in production methods to express an OLD-35 protein.

In further embodiments, the present invention provides for methods of promoting terminal differentiation in a cell comprising introducing, into the cell, an old-35 nucleic acid operatively linked to a promoter element, such that an amount of OLD-35 protein is produced sufficient to increase at least one indicia of terminal differentiation. Examples of such indices include growth suppression, as evaluated by FACS analysis, or by MTS or MTT assays, morphological changes including dendrite extension, and expression of terminal differentiation markers, which in the case of melanoma cells would include synthesis of melanin. In the case of most mammalian cells, indicia of growth suppression would also include a reduction in the expression of telomerase and c-myc, alterations in the level of expression of one or more of the CDKIs (e.g. p15, p16, p21 and p27), increases in the level of expression of double-stranded RNA-dependent protein kinase R (PKR) or the DNA damage-inducible gene GADD153, as well as phosphorylation of eukaryotic initiation factor 2α (eIF2α). In a preferred non-limiting embodiment of the invention, the old-35 nucleic acid has a sequence as set forth in SEQ ID NO:1. In a related non-limiting embodiment, the OLD-35 protein expressed has a sequence as set forth in SEQ ID NO:2.

The present invention also provides for methods of promoting senescence in a cell comprising introducing, into the cell, an old-35 nucleic acid operatively linked to a promoter element, such that an amount of OLD-35 protein is produced sufficient to increase at least one indicia of senescence. Examples of such indices include growth suppression, morphological changes including enlargement of cell bodies, expression of the CDKIp21, reduced telomerase activity, and positive staining for expression of senescence-associated β-galactosidase (SA-β-gal). In a preferred non-limiting embodiment of the invention, the old-35 nucleic acid has a sequence as set forth in SEQ ID NO:1. In a related non-limiting embodiment, the OLD-35 protein expressed has a sequence as set forth in SEQ ID NO:2.

The present invention also provides for methods of reversing, partially or completely, a transformed phenotype of a cell comprising introducing, into the cell, an old-35 nucleic acid operatively linked to a promoter element, such that an amount of OLD-35 protein is produced sufficient to decrease at least one indicia of the transformed phenotype. Examples of such indices include the ability to form colonies in soft agar, lack of contact inhibition, an undifferentiated phenotype, increased rate of cell division, and expression of transformation-associated molecules. In a preferred non-limiting embodiment of the invention, the old-35 nucleic acid has a sequence as set forth in SEQ ID NO:1. In a related non-limiting embodiment, the OLD-35 protein expressed has a sequence as set forth in SEQ ID NO:2.

The present invention also provides for methods of decreasing the rate of cell proliferation and/or DNA synthesis comprising introducing into the cell an old-35 nucleic acid operatively linked to a promoter element, such that an amount of OLD-35 protein is produced sufficient to decrease the rate of cell proliferation. In a preferred non-limiting embodiment of the invention, the old-35 nucleic acid has a sequence as set forth in SEQ ID NO:1. In a related non-limiting embodiment, the OLD-35 protein expressed has a sequence as set forth in SEQ ID NO:2.

The present invention also provides for methods of inducing cellular apoptosis comprising introducing into the cell an old-35 nucleic acid operatively linked to a promoter element, such that an amount of OLD-35 protein is produced sufficient to produce cellular apoptosis or to alter at least one determinant of apoptosis. Examples of such determinants include the level of expression of the anti-apoptotic bcl-2 and bcl-xl proteins and the pro-apoptotic bax protein. In a preferred non-limiting embodiment of the invention, the old-35 nucleic acid has a sequence as set forth in SEQ ID NO:1. In a related non-limiting embodiment, the OLD-35 protein expressed has a sequence as set forth in SEQ ID NO:2.

The present invention further provides for methods of promoting terminal differentiation and/or senescence, reversing the transformed phenotype, decreasing the rate of cell proliferation and/or DNA synthesis, and/or inducing apoptosis comprising introducing, into a cell, an effective amount of an OLD-35 protein. Preferably the OLD-35 protein has an amino acid sequence as set forth in SEQ ID NO:2. The protein may be introduced directly, via a carrier molecule, via a microparticle, via a liposome, or by other methods known in the art.

### 5.3 The Old-35 Promoter and its Variants

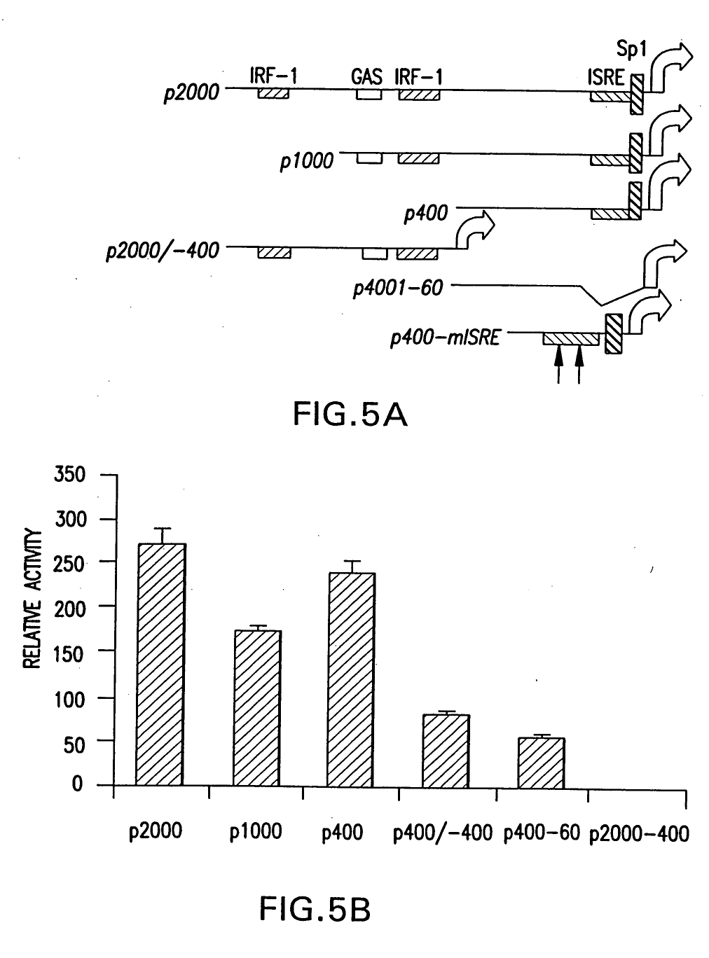

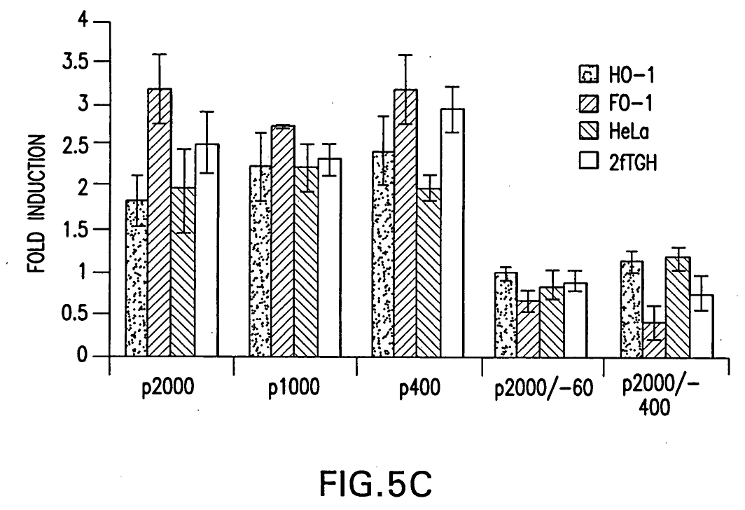

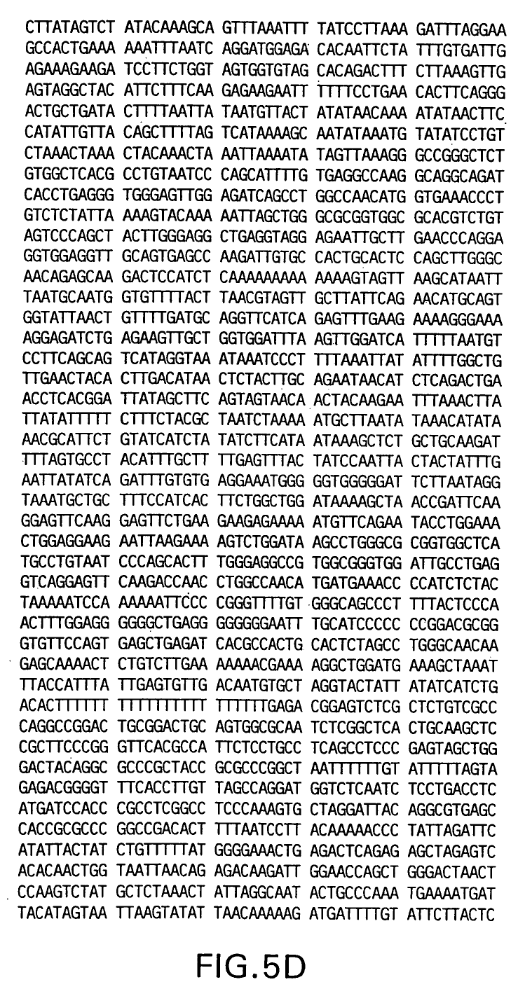

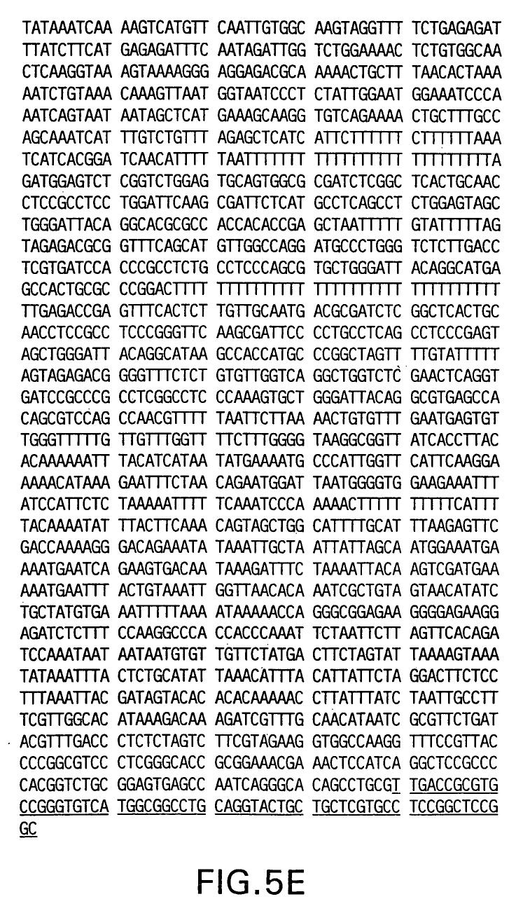

The present invention provides for the promoter of the human old-35 gene and variants thereof. The term “variants” includes fragments, deletion mutants, insertional mutants, point mutants, substitution mutants, nucleic acid molecules comprising one or more modified nucleic acid, etc. The wild-type old-35 promoter and variants thereof are collectively referred to as “old-35 promoters.” Preferably variants are at least 85 percent, preferably at least 90 percent homologous to a nucleic acid molecule having a sequence set forth in SEQ ID NO:4 (FIG. 5D) and/or hybridize to a nucleic acid molecule having a sequence set forth in SEQ ID NO:4 (FIG. 5D), or its complementary strand, under stringent conditions for detecting hybridization of nucleic acid molecules as set forth in “Current Protocols in Molecular Biology”, Volume I, Ausubel et al., eds. John Wiley: New York N.Y., pp. 2.10.1-2.10.16, first published in 1989 but with annual updating, wherein maximum hybridization specificity for DNA samples immobilized on nitrocellulose filters may be achieved through the use of repeated washings in a solution comprising 0.1-2×SSC (15-30 mM NaCl, 1.5-3 mM sodium citrate, pH 7.0) and 0.1% SDS (sodium dodecylsulfate) at temperatures of 65-68° C. or greater. For DNA samples immobilized on nylon filters, a stringent hybridization washing solution may be comprised of 40 mM NaPO₄, pH 7.2, 1-2% SDS and 1 mM EDTA. Again, a washing temperature of at least 65-68° C. is recommended, but the optimal temperature required for a truly stringent wash will depend on the length of the nucleic acid probe, its GC content, the concentration of monovalent cations and the percentage of formamide, if any, that was contained in the hybridization solution.

Deletion mutants of the old-35 promoter preferably hybridize to a nucleic acid molecule having a sequence as set forth in SEQ ID NO:4 under stringent conditions.

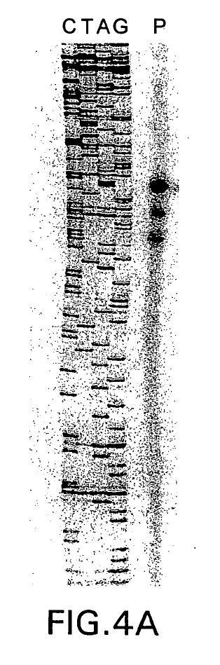

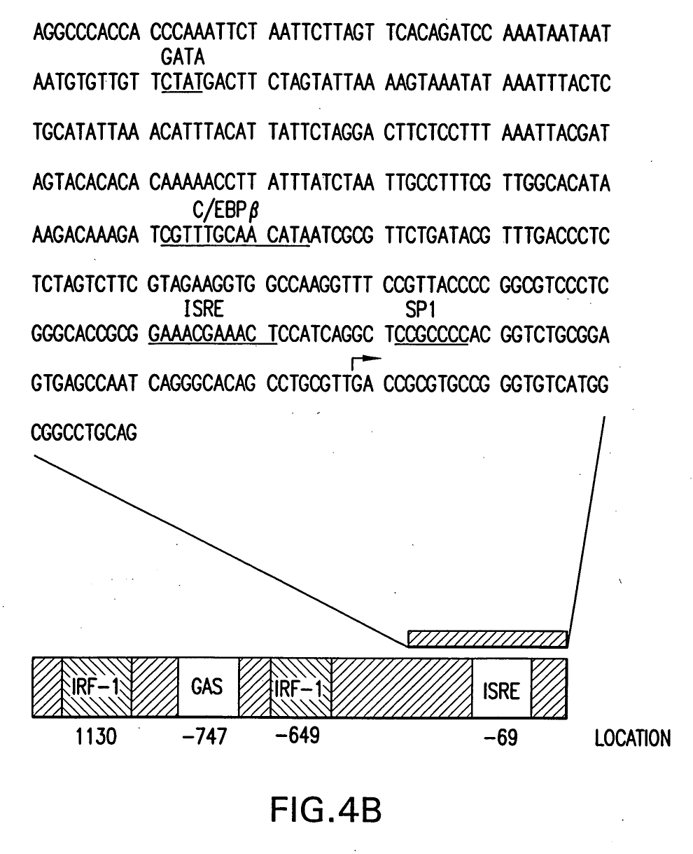

In specific, non-limiting embodiments, the invention provides for an old-35 promoter and variants thereof as described in section 6 below. In one embodiment, an old-35 promoter is contained in p2000, as depicted in FIG. 5A, and the sequence of which is set forth as bold face text in FIG. 5D. Specific examples of old-35 promoter variants include p1000, p400, p2000/−400, p400/−60 and p400-mISRE, as depicted in FIG. 5A and described in section 6 below. The nucleic acid sequence of p400 is depicted in FIG. 4B and SEQ ID NO:3.

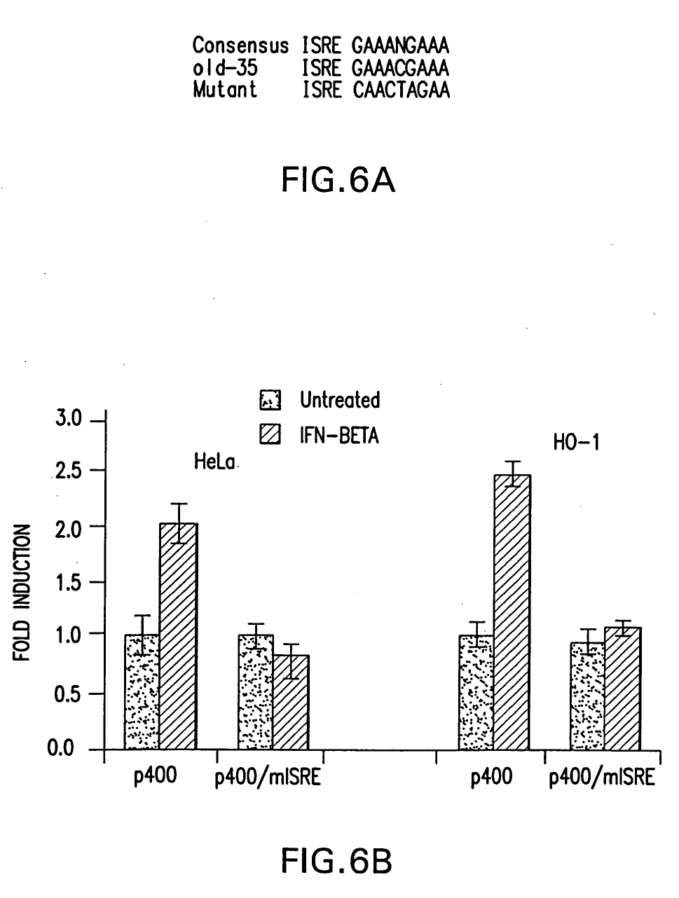

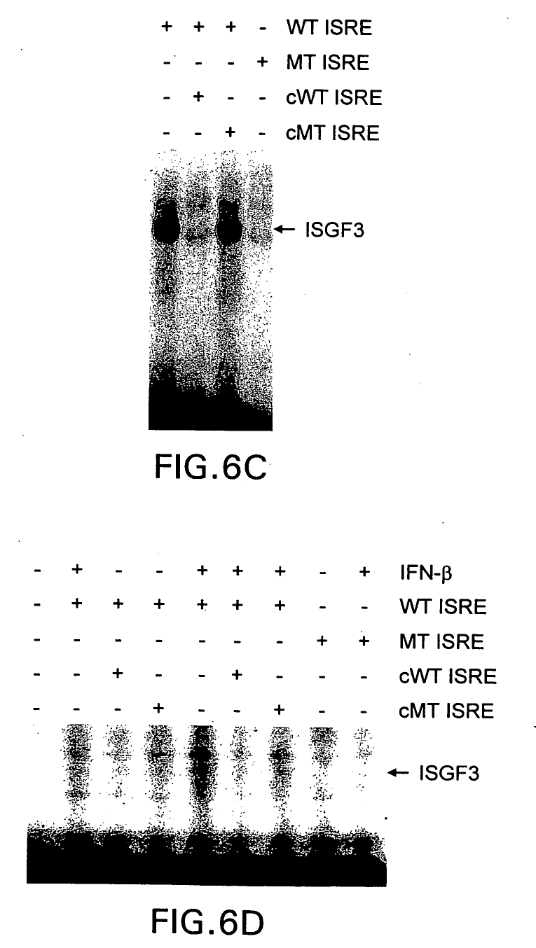

The present invention further provides for isolated nucleic acid molecules comprising subregions of an old-35 promoter, including but not limited to an old-35 Interferon-Stimulated Response Element (“ISRE”) having a sequence as set forth in SEQ ID NO:6 and depicted in FIG. 6A and a mutant ISRE having a sequence as set forth in SEQ ID NO:7 and depicted in FIG. 6A.

Data demonstrating that IFN-β was more effective in upregulating p400 than the p2000 construct (see section 6 below) indicate that one or more repressor element(s) is present in the p2000 construct. It may be desirable to omit this one or more repressor element from constructs intended to optimize promoter activity. One non-limiting example of an old-35 promoter variant lacking the repressor is the p400 variant.

The present invention provides for an old-35 promoter operatively linked to a gene of interest which, when introduced into a suitable host cell, results in the transcription of the gene of interest and preferably in the expression of a protein encoded by the gene of interest. The gene of interest may be an old-35 gene or may be another gene (a “heterologous”) gene. Examples of non-old-35 genes of interest include but are not limited to reporter genes, such as the genes encoding green fluorescent protein, 13-glucuronidase, β-galactosidase, luciferase, and dihydrofolate reductase, genes which increase cell proliferation and/or inhibit terminal cell differentiation and/or senescence such as the c-myc or telomerase genes, genes which decrease cell proliferation and/or promote terminal cell differentiation and/or senescence such as the p21. p53, p27, p16(ink), mda-7 and PML genes, genes which have an antiviral effect such as those encoding intereforons or PKR, and genes that increase cellular proliferation, such as c-myc, c-fos, raf, ras, and Alk.

Nucleic acid molecules comprising an old-35 promoter, for example operatively linked to a gene of interest, may optionally be incorporated into a vector molecule suitable for replication and/or expression. Suitable vectors include those described in Section 5.1.

### 5.4 Uses of the Old-35 Promoter and its Variants

The present invention provides for assay systems for identifying agents that modulate old-35 promoter activity. An agent that increases old-35 promoter activity may be used to promote terminal cell differentiation and/or cellular senescence and/or to decrease the rate of proliferation of a cell, for example a tumor cell, for example a tumor cell in a subject in need of such treatment.

An agent that decreases old-35 promoter activity may be used to inhibit terminal cell differentiation and/or cellular senescence and/or to increase the rate of proliferation of a cell. Such agents may be useful in the treatment of disorders of premature senescence (for example progeria), for the preservation of viable cells and tissues in culture, for treatment of cells prior to transplant, and for other applications where maintenance of cell viability and/or plasticity is desirable.

An assay system of the invention comprises a cell containing an old-35 promoter (which may be a wild-type old-35 promoter or a variant thereof) operatively linked to a gene of interest wherein if the gene of interest is transcribed, a detectable product (nucleic acid or protein) is directly or indirectly produced. An example of indirect production is where the gene of interest induces the expression of a second gene, the product of which is detectable. The gene of interest may be virtually any gene which directly or indirectly produces a detectable product. In specific non-limiting embodiments of the invention, the gene of interest is a reporter gene known and used as such in the art, such as, but not limited to, green fluorescent protein, luciferase, β-glucuronidase, β-galactosidase, etc. The assay system of the invention may further comprise a test agent wherein the test agent is to be evaluated for its effect on old-35 promoter activity.

The present invention further provides assay methods comprising exposing a cell containing an old-35 promoter linked to a gene of interest to a test agent and determining the effect of the test agent on the production of a direct or indirect product of the gene of interest. Preferably, in a parallel experiment, the production of the product is determined where the cell has not been exposed to a test agent. Exposure of the cell to the test agent may be continuous or for a limited period of time. Indices of production include but are not limited to concentration of the product and rate of its accumulation or destruction. Where the product is a direct product of the gene of interest (for example, an RNA transcribed from the gene or the protein encoded by it), an increase in product correlates with an increase in old-35 promoter activity and a decrease in product correlates with a decrease in old-35 promoter activity. Where the product is indirect, interpretation will depend on the relationship between the gene of interest and the indirect product to be measured.

The ability of agents to activate or inhibit promoter activity in constructs either containing a repressor (such as p2000) or lacking a repressor (such as p400) may be compared in order to identify agents that modulate promoter function.

For example, an agent that inhibits repressor activity may be identified and used in methods of enhancing old-35 activity, either alone or together with another promoter activating agent. Alternatively, an agent that increases repressor activity may be used to inhibit senescence and/or terminal differentiation and/or to augment cell proliferation.

A repressor-enhancing agent may also be used in conduction with an old-35 promoter activating agent, for example, where the promoter activating agent is used to control the proliferation of cells in a tumor but the repressor enhancing element is used to protect the viability of non-cancerous cells. Such selective activation/repression may be achieved by local administration or other known methods of targeting molecules.

Removal of the region most proximal to the transcription initiation start site, which contains ISRE and Sp1 elements (as in p400/−60), was observed to abrogate the ability of IFN-β to enhance old-35 expression (see section 6, below, and FIG. 5B). Agents that modulate interferon inducibility may be identified using assay systems that compare the effects of a test agent on (i) the activity of the old-35 promoter or a variant, such as p400, which comprises an interferon-inducible element and (ii) the activity of an old-35 promoter variant lacking an interferon-inducible element, such as p400/−60. Activating agents identified by such an assay may be used to increase promoter activity by either augmenting interferon inducibility or by a mechanism complementary to interferon induction; such agents may be particularly useful as adjuvants of interferon therapy.

### 6. EXAMPLE

### Cloning, Expression Regulation and Genomic Characterization of Old-35

6.1 Materials and Methods

Cell lines and culture conditions. HO-1 is a melanotic melanoma cell line established from a metastatic inguinal lymph node lesion from a 49 year-old female (Fisher et al., 1985, J. Inter. Res. 5: 11-22). WM35 was derived from a radial growth phase primary melanoma (Herlyn, 1990, Cancer Metastasis Rev 9: 101-112). C8161 is a highly metastatic amelanotic human melanoma cell line derived from an abdominal wall metastasis (Welch et al., 1991, Int. J. Cancer 47: 227-237). C8161 clones containing a normal human chromosome 6, designated C8161-6.3, were established as described in Welch et al., 1994, Oncogene 9: 255-262. Additional human melanoma cell lines isolated from patients with metastatic melanomas included FO-1, MeWo, 3S5 (a non-metastatic variant of MeWo), WM239, SK-MEL wt p53 (SK-MEL 470) and SK-MEL wt p53 (SK-MEL 110) (Graham et al., 1991, Cancer Inmunol. Immunother 32: 382-390; Jiang et al., 1995, Oncogene 11: 2477-2486; Fisher et al., 1985, J. Inter. Res. 5: 11-22; Herlyn, 1990, Cancer Metastasis Rev 9: 101-112). 2ftGH cells are human HT1080 fibrosarcoma cells transfected with a bacterial gpt gene controlled by an IFN inducible promoter (Pellegrini et al, 1989, Mol. Cell. Biol. 9: 4605-4612). U1, U3, U4, and U5 are derived from 2ftGH (Pellegrini et al, 1989, Mol. Cell. Biol. 9: 4605-4612). GM01379A is a human fibroblast cell line derived from a lung biopsy of a 12 year old male (Coriell Repository, Camden, N.J.). AG0989B are human skin fibroblasts derived from a patient with progeria, Hutchinson-Gilford syndrome (Coriell Repository, Camden, N.J.). HeLa cells were derived from a patient with cervical carcinoma and were obtained from the ATCC. MCF-7, MDA-MB-157 and MDA-MB-231 human breast carcinoma cell lines and Saos2 human osteosarcoma cells were obtained from the ATCC. All cells were grown in Dulbecco's modified Eagle's medium (DMEM) supplemented with 10% fetal bovine serum and penicillin/streptomycin (100 U/100 μg/ml) at 37° C. in a 5% CO₂/95% air humidified incubator. Prior to all treatments, cells were cultured in fresh DMEM and exposed to the indicated compound(s) at the concentrations specified.

RNA extraction, Northern blotting and primer extension analysis. Total RNA was purified from cells using the RNA easy kit (Qiagen). For Northern blotting, ten μg of total RNA was resolved in 1% agarose gel with 2% formaldehyde and transferred to Nylon membranes (Hybond-N™). The quality of RNA was determined by examining intact 28S and 18S rRNA bands. XhoI fragment of old-35 cDNA (1.5-kb) and 0.7 kb fragment of gapdh were labeled with [α-³²P]dCTP using a Multiprime Labeling Kit™ (Roche). RNA levels were quantitated in comparison with gapdh. The membrane was hybridized in ExpressHyb® according to the manufacturer's recommendations (Clontech) with old-35; the blots were stripped and reprobed with gapdh. Primer extension analysis was performed as described in Su et al., 2000, Oncogene 19: 3411-3421.

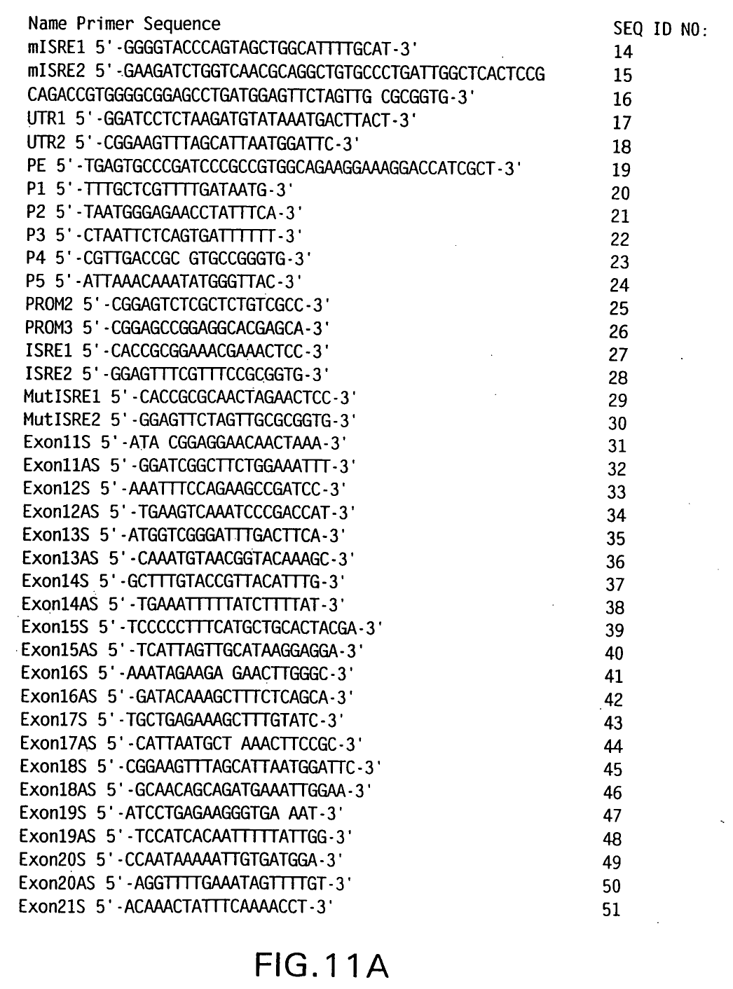

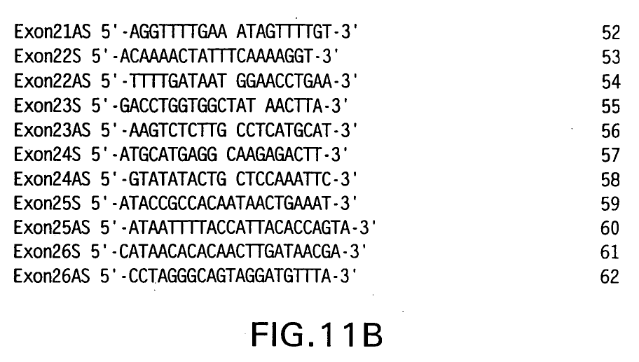

Library screening and cloning of old-35. Using a subtractive hybridization approach, a library enriched in genes from different stages in terminal differentiation of human melanoma cells, referred to as a differentiation induction subtraction hybridization (DISH) library, was constructed as described in Jiang and Fisher, 1993, Mol. Cell. Diff. 1: 285-299 and Huang et al., 1999, Oncogene 18: 3546-3552. To prepare the senescent probe for library screening, AG0989B progeria cells were cultured until their population doubling times increased to more than three weeks. When collected, in addition to profound morphological changes, the cells stained positive for Senescence Associated β-galactosidase (SA-β-gal), which is an established marker for determination of the senescent state (Dimri et al., 1995, Proc Natl. Acad. Sci. USA 92: 9363-9367). Three μg of poly(A)₊RNA, extracted using Poly(A) Pure kit (Ambion), was reverse transcribed using Superscript reverse transcriptase (Gibco BRL) in the presence of [α-³²P]dCTP according to the manufacturers suggestions (Gibco BRL). The library was screened as described in Jiang and Fisher, 1993, Mol. Cell. Diff. 1: 285-299. Positive clones showing upregulation in IFN-β +MEZ treated cells were sequenced to establish their identity with ABI automatic sequencer. One of the identified clones was named old-35. The full-length old-35 cDNA was cloned using standard and modified RACE protocols in both the 5′ and the 3′ directions (Jiang et al., 1995, Oncogene 11: 2477-2486; Jiang et al., 1995, Oncogene 10: 1855-1864) (FIG. 1). The 5′ region of old-35 was cloned using an old-35 specific primer P1 (FIG. 11) from IFN-β treated HO-1 cells using a modified RACE protocol. The 3′ region of old-35 was cloned using the 3′ RACE procedure with gene specific nested primers P2 and P3 (FIG. 11) and dT primer, yielding an ˜400-bp product. 5′ and 3′ RACE products were cloned into pT-Adv vector (Clontech) and sequenced with an ABI automatic sequencer. After sequence confirmation, primers P4 and P5 (FIG. 11) were designed in the 5′ and 3′ region of the cDNA respectively and the full-length old-35 cDNA was obtained using RT-PCR with total RNA isolated from IFN-β treated HO-1 cells. To identify the larger ˜4.3-kb old-35 variant a bioinformatics approach was used. The DEST database was searched for clones longer than 2.6-kb that contained an old-35 signature (www.ncbi.nlm.nih.gov/blast). An ˜4.3-kb clone of old-35 cDNA was identified and obtained from the ATCC (#213524).

Genomic mapping and exon/intron analysis. A BLAST search was used to screen the HTGS genomic database with the old-35 cDNA. Two BAC clones were identified which contained sequences homologous to old-35. BAC RP11-327M20 and RP11-152018 were obtained from Research Genetics and analyzed by sequencing with gene specific primers (FIG. 11) using an ABI automated sequencer to establish exon-intron boundaries and to determine exon size. The intron length was established using PCR and BAC RP11-152018 as a template. The exon-intron boundaries for exon 1 to exon 10 (FIG. 12) were derived from available sequences in HTGS database (www.ncbi.nlm.nih.gov).

Cloning of the old-35 promoter, construction of deletion mutants and old-35 promoter analysis. A ˜2-kb fragment of the old-35 promoter, containing the ISRE element and the first intron up to the ATG, was amplified using PCR with PROM3 and PROM2 (FIG. 11) using RP11-152018 BAC containing the old-35 genomic sequence as a template. The fidelity of the PCR reaction was confirmed by sequencing and the amplified fragment was cloned into PCR ready pT-Adv vector (Clontech). The insert was released by KpnI and XhoI digestion and cloned into the pGL3-basic vector (Promega). This plasmid, designated as p2000, was used as a parental plasmid to generate the deletion constructs. To construct p1000, the parental plasmid was digested with SmaI and XhoI. The released insert was cloned into pGL3-basic in the SmaI and XzoI sites. p400 was created by restriction digest of the parental plasmid with BglII and HindIII and cloned into pGL3-basic at the BglII and HindIII site. p2000/−400 was created by BglII digestion, which released a 400-bp fragment which was then religated to the remainder of the construct. mISRE was constructed using a PCR method by amplifying the p400 with mISRE1 and mISRE2. The PCR amplified fragment was digested with BglII and cloned into pGL3-basic. P400/−60 was created by SacII (promoter region) and KpnI (plasmid region) digestion of the p400 construct, blunt ending and religation. The 2.6-kb old-35 UTR was amplified using RT-PCR with dT primer and Superscript (Gibco BRL), and UTR1 and UTR2 primers (FIG. 11) and Advantage cDNA Polymerase (Clontech), respectively. The amplified fragment was digested with BglII and BamHI and cloned into the BamHI site in p400 in the correct orientation (p400 UTR).

Luciferase assays. Luciferase assays were performed as described in Su et al., 2000, Oncogene 19: 3411-3421. HO-1, FO-1, HeLa or 2ftGH cells were transfected in 6-well plates by the Superfect transfection method (Qiagen). Five μg of the reporter plasmid DNA was used for each transfection. Transfected cells were allowed to recover overnight. The next day, transfected cells were treated with IFN-β (2000 units/ml) for 7 h. Cells were harvested, lysed, and luciferase activity was measured according to the manufacturer's suggestions (Promega). All experiments were done in triplicate and repeated at least 3 times.

Electrophoretic mobility shift assay (EMSA). ISRE1 and ISRE2 primers were used to generate ISRE DNA and mutISRE1 and mutISRE2 were used to produce the mutated ISRE designated as mutISRE. Gel shift assays were performed as described in Dimri et al., 1995 (Proc Natl. Acad. Sci. USA 92: 9363-9367).

6.2 Results

Cloning of the old-35 cDNA. Screening of a subtracted library, enriched for genes upregulated during the process of terminal differentiation with a senescent probe derived from progeria skin fibroblasts, identified a differentially expressed 600-bp EST, old-35 (clone-35). The original 600-bp fragment of old-35 contained an internal region of old-35 cDNA and lacked 3′ and 5 ′ flanking sequences. The 5′ and 3′ region of old-35 was cloned from IFN-β treated HO-1 cells using a modified RACE protocol (FIG. 1B). This protocol resulted in the cloning of a 2629-bp cDNA fragment (FIG. 1A; the 3′ region of this variant ends in the sequence AAATTAC). Northern blotting analysis indicated that the old-35 EST hybridized to two mRNA species of ˜4.0-kb and 2.6-kb in IFN-β treated HO-1 RNA samples (FIG. 1C). Since the cDNA cloned from IFN-β treated HO-1 cells was 2.6-kb, we attempted to identify the ˜4-kb variant using a bioinformatics approach with a blast search of the dbest database. A 4.3-kb clone was purchased from ATCC and sequenced. Comparison of the old-35 cDNA clone and the ATCC clone #213524 revealed that they were 100% homologous. However, the 4.3-kb clone had a longer 3∝ UTR, possibly due to differential polyadenylation. To determine whether the upper band on the old-35 Northern blot corresponded to the ˜4.3-kb old-35 hybridizing mRNA species, a Northern blot containing total RNA from IFN-β treated HO-1 cells was probed with either the coding region of the old-35 gene or with the 3′ UTR of the longer ˜4.3-kb variant (FIG. 1C). When this Northern blot was probed with a coding region probe, ˜2.6-kb and ˜4-kb bands were detected. On the other hand, when the 3′ UTR probe was used to probe this Northern blot, only the upper ˜4-kb band was detected. These results indicate that the ˜4.3-kb mRNA is a variant of old-35. The coding region of the ˜4.3-kb and 2.6-kb old-35 mRNAs exhibit 100% identity, but the ˜4.3-kb variant has a longer 3′ UTR.

Sequence analysis of old-35. The two variants of old-35 are 2629-bp and 4331-bp long and they contain an ORF that extends from 53- to 2404-bp, encoding a protein of 783 amino acids with a predicted Mᵣ of 86 kDa with a pI of 7.87. The ORF starts at the first AUG codon. Although A₋₃ in Kozak consensus sequence (AXXaugG) is not conserved, G₊₄ is conserved (Kozak, 1996, Mamm Genome 7(8): 563-574). Sequence analysis of old-35 revealed that this cDNA (˜2.6-kb) contains a less frequently used polyadenylation site (AUUAAA) (found in only ˜10% of cDNAs) (Manley, 1988, Biochim Biophys Acta 950: 1-12). A canonical polyadenylation site was not detected in the ˜4.3-kb variant (FIG. 1A).

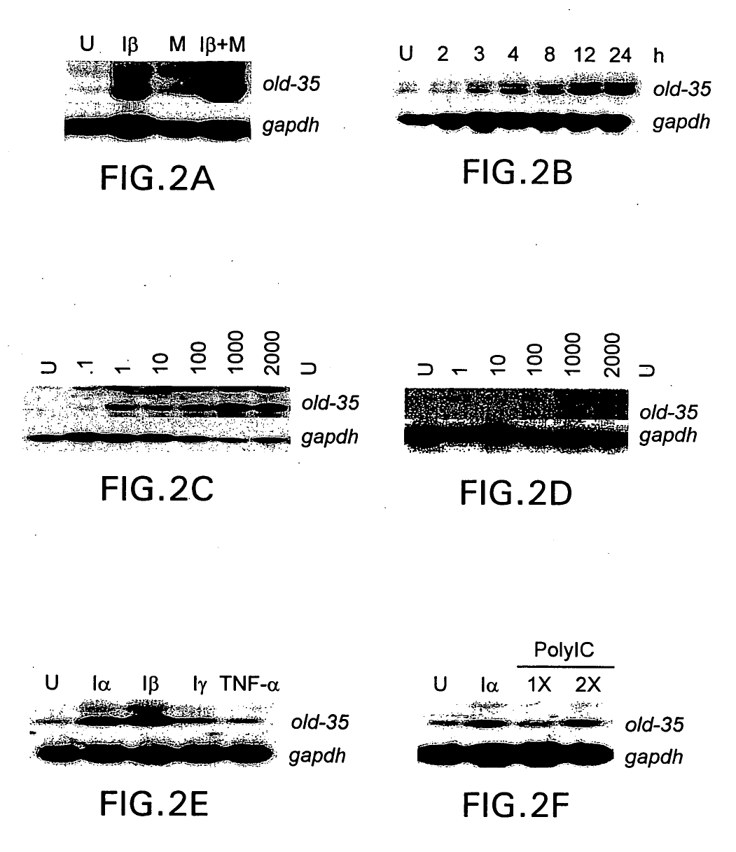

Old-35 is an early Type IIFN-inducible gene. Since old-35 was cloned as a result of screening a temporally spaced IFN-β+MEZ treated human melanoma subtracted library, it was considered important to establish whether old-35 expression was induced by IFN-β, MEZ or the combination of IFN-β+MEZ. Treatment of HO-1 with IFN-β (2000 units/ml) stimulated old-35 expression in HO-1 cells. Although MEZ (10 ng/ml) did not affect old-35 expression, the combination of IFN-β+MEZ (2000 units/ml+10 ng/ml) stimulated old-35 to a higher extent than IFN-β alone (FIG. 2A). Time-course and dose-response experiments were performed in HO-1 cells to determine the temporal kinetics of old-35 induction by IFN-β and the concentration of IFN-β capable of inducing old-35 expression, respectively. Old-35 was induced within 3 h by IFN-β (2000 units/ml) (FIG. 2B). Since IFN-β induces growth suppression in HO-1 cells at 2000 units/ml, it was important to establish whether up-regulation of old-35 could occur in the absence of growth suppression. Old-35 expression was induced in HO-1 cells with as little as 1 unit/ml of IFN-β (FIG. 2C), a dose of IFN that is not growth inhibitory, suggesting that IFN modulates expression of old-35 in the absence of growth suppression. When HO-1 cells were treated for 24 h with IFN-β, a higher concentration (1000 to 2000 units/ml) of IFN-β was required to induce old-35 expression (FIG. 2D). Treatment of HO-1 cells with IFN-α also resulted in significant up-regulation of old-35 in HO-1 cells, whereas expression of old-35 was marginally stimulated by IFN-γ and no detectable or consistent induction occurred with TNF-α (FIG. 2E). Double-stranded RNA, poly inosinic-cytidylic acid (PolyIC), a known inducer of IFN-α and IFN-β genes also stimulated old-35 expression (FIG. 2F).

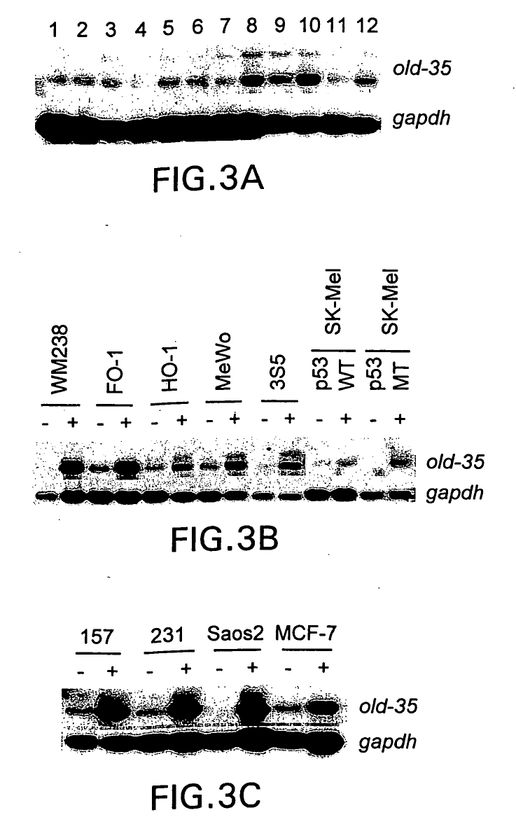

Since old-35 was cloned from HO-1 cells, a metastatic human melanoma cell line, old-35 expression was examined in additional melanoma cell lines. The steady state de novo expression of old-35 was comparable in FO-1, HO-1, MeWo and 3S5 (nonmetastatic variant of MeWo) human melanomas with reduced de novo expression in the WM238, SK-Mel p53mt (mutant p53) and SK-Mel p53wt (wild type p53) melanoma cell lines (FIG. 3A, B). However, when treated with IFN-β, expression of old-35 was elevated or induced to variable extents in all of the melanoma cell lines.

To test for upregulation of old-35 by IFN-β in cells other than melanoma, HeLa (human cervical carcinoma), human skin fibroblasts, MDA-MB-157 (p53-null), MDA-MB-231 (mut-p53) and MCF-7 (wt-p53) (human breast carcinoma) and Saos2 (p53- and Rb-null) (human fibrosarcoma) cells were treated with IFN-β for 18 h and old-35 mRNA levels were determined (FIG. 3A). The level of old-35 was induced to a greater extent in HO-1 (FIG. 3A, lane 7, 8) and human fibroblasts (FIG. 3A, lane 11, 12) than in HeLa (FIG. 3A, lane 9, 10). It was also apparent that de novo old-35 expression was higher in HeLa cells as compared to the other cell lines tested (FIG. 3A, lane 9).

These experiments document differential regulation of old-35 expression by different cytokines, with Type I IFNs (IFN-α/IFN-β) being the most active cytokines tested in inducing old-35 expression in HO-1 cells. In addition, expression of wt-p53 or Rb is not required for induction of old-35 by IFN-β.

Identification of the old-35 transcription start site. To determine the transcription initiation start site of the old-35 gene, the primer extension method was performed. Labeled antisense primer was hybridized to total RNA from IFN-β treated HO-1 cells and the extension products were separated on a sequencing gel (FIG. 4A). This experiment demonstrated that the major transcript is being initiated from a thymidine residue located 19-bp upstream of the ATG start codon. Accordingly, this base was designated as +1-bp and extended the 5′-end of the previously cloned old-35 cDNA by 5-bp (FIG. 4A, B). Two additional minor bands were observed during the primer extension procedure and might be due to high GC content of the 5′-region or may represent alternative transcriptional start sites.

The old-35 promoter responds to IFN through its ISRE element. To further characterize the influence of IFN on old-35 expression, we cloned and studied the old-35 promoter region to identify sequence elements and trans-acting factors involved in IFN-induced old-35 expression. A bioinformatics analysis of the old-35 promoter was initiated by examining its sequence with MatInspector V2.2 for recognizable transcription elements. This analysis indicated that the old-35 promoter region does not contain defined TATA or CAAT elements, but it possesses an IFN-stimulated response element (ISRE), a GAS element, two IRF-1 binding and C/EBPβ sites which play a role in initiating transcription of various IFN stimulated genes (ISGs) (Roy et al., 2000, J. Biol. Chem. 275: 12626-12632) (FIGS. 4B and 5A). The old-35 promoter also contains a GC-rich Sp1 site, which plays an important role in potentiating transcription from TATA-less promoters (Azizkhan et al., 1993, Crit. Rev. Eukaryot. Gene Expr. 3: 229-254).

Since old-35 is expressed in HO-1 cells at low levels, it was important to determine the effect of various deletions on the activity of the old-35 promoter. Deletions of the distal region did not affect the activity of the old-35 promoter (p2000, p1000) (FIGS. 5A and 5B). On the other hand, deletion of the proximal region from p2000 (p2000/-400) completely abolished promoter activity. Examination of the p400 sequence for potential elements that might contribute to old-35 transcriptional activation identified one ISRE and one SpI element. Deletions of these elements from p400 (p400/−60) resulted in a three-fold reduction in old-35 promoter activity suggesting that these two elements are important, however they were not sufficient to produce maximum transcription of old-35 in these cells (FIG. 5B).

To distinguish between Sp1 and ISRE dependent activation, a point mutation was engineered in the ISRE in the p400 construct (p400/mISRE). As with the p400/−60 construct, a point mutation in the ISRE resulted in a threefold reduction in the activity of the old-35 promoter suggesting that it is the ISRE and not the Sp1 in p400/−60 that contributes to the observed three-fold reduction.

Since old-35 is an IFN stimulated gene, it was considered relevant to determine whether the old-35 promoter was responsive to IFN-β treatment. IFN-βtreatment of four different cell lines including HO-1, FO-1, HeLa and 2fTGH resulted in a 2-3 fold upregulation of the p2000 old-35 promoter construct (FIG. 5C). It is worth noting that IFN-β was more effective in upregulating p400 than the p2000 construct indicating that a repressor element(s) may be present in the p2000 construct. The pattern of IFN-β mediated stimulation of the various old-35 constructs was similar in the four cell lines. However, upregulation of the old-35 promoter was highest in FO-1 and lowest in HeLa cells (FIG. 5C). Moreover, a similar pattern of old-35 upregulation by IFN-β was observed by Northern blotting (FIGS. 3A and 3B). Removal of one or both IRF-1 sites did not decrease the ability of IFN-β to stimulate old-35 transcription. Similarly, when the GAS sequence was deleted, as in p400, luciferase activity was not decreased (FIG. 5C). In contrast, removal of the region most proximal to the transcription initiation start site, which contains ISRE and Sp1 elements (as in p400/−60), abrogated the ability of IFN-β to enhance old-35 expression (FIG. 5B).

Since it is recognized that the ISRE plays an important role in the activation of ISGs, the p400/mISRE construct was tested (FIG. 6A). Introduction of two point mutations in the ISRE element extinguished IFN-β stimulation of the old-35 promoter and decreased constitutive promoter activity in these cells by ˜3-fold, thereby implicating the ISRE as the major IFN-responsive element in the old-35 promoter (FIG. 6B).

To confirm that the loss in IFN responsiveness was due to lack of ISGF3 complex binding to the ISRE, oligonucleotides containing either the wild type or mutant ISRE (FIG. 6A) were examined by electrophoretic mobility shift (gel shift) assays (EMSA) (FIG. 6B). When the wild-type ISRE was incubated with nuclear extracts from HeLa (FIG. 6C) or HO-1 (FIG. 6D) cells, a shifted band was observed indicating the formation of a DNA-protein complex. This complex was specific since competition was observed when incubating with wild type cold ISRE DNA and not with mutant ISRE DNA (FIGS. 6C and 6D). As anticipated, IFN-β treatment enhanced formation of the ISRE-ISGF3 complex (FIG. 6D). This complex was also decreased following incubation with unlabeled wild type oligonucleotides, but not with the mutant oligonucleotides. Additionally, the mutant ISRE could not bind to DNA, since no gel shifted complex was observed (FIGS. 6C and 6D). Overall, these experiments demonstrate that constitutive and IFN-β mediated expressions of old-35 are controlled by different elements. IFN-β upregulation of the old-35 promoter was dependent upon complex formation between ISGF3 and the ISRE element. On the other hand, since deletion of the ISRE did not completely abrogate the constitutive activity of old-35 in untreated HO-1 cells, it is evident that additional elements between −400-bp and −60-bp are important for this activity.

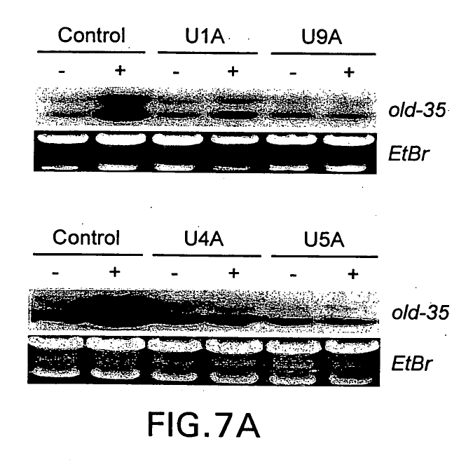

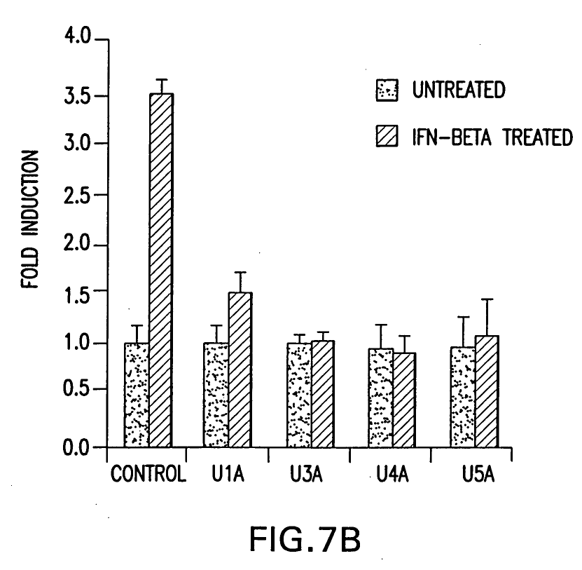

Old-35 expression is dependent on JAK/STAT signaling. Experiments demonstrating that old-35 is an early Type I IFN inducible gene and transcription is dependent on ISGF3-ISRE complex formation strongly suggested the involvement of JAK/STAT signal transduction in old-35 transcriptional activation following IFN-β treatment. To test this possibility, we utilized a series of human fibrosarcoma cells derived from 2fTGH cells which are technically wild type in terms of IFN signaling while its derivatives U1, U3, U4, and U5 are defective in Tyk2, STAT1, JAK1 and IFNAR1, respectively (Stark and Gudkov, 1999, Hum. Mol. Genet. 8: 1925-1938). To test whether old-35 expression is affected by specific mutations in IFN signaling, Northern blots containing total RNA from 2fTGH and its derivatives were probed with an old-35 cDNA. These results showed that old-35 is upregulated by IFN-β treatment only in the control (2fTGH) and not in the mutant cell lines (FIG. 7A). Similarly, the old-35 promoter (p1000) was stimulated by IFN-β in 2fTGH cells and not in the 2fTGH JAK/STAT mutant cells (FIG. 7B) indicating that the JAK/STAT pathway is crucial for old-35 activation.

Old-35 3′ UTR does not contribute to regulation of old-35 by IFN-β. Gene expression is regulated at multiple levels, including transcription and mRNA stability. Early response genes, such as cytokines, lymphokines and proto-oncogenes are regulated by a cis-acting adenylate/uridylate-rich element (ARE) found in the 3′ untranslated region (UTR) of their mRNA (Chen and Shyu, 1995, Trends Biochem. Sci. 20: 465-470).

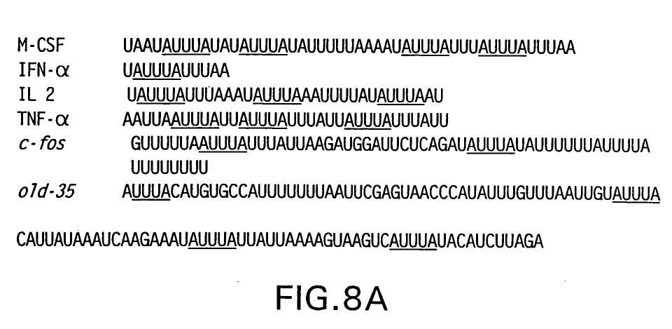

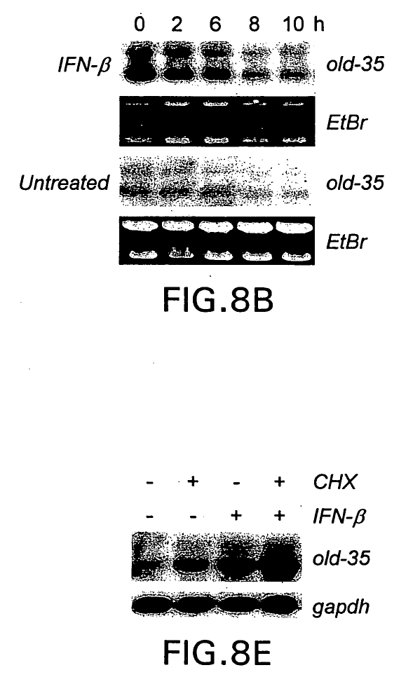

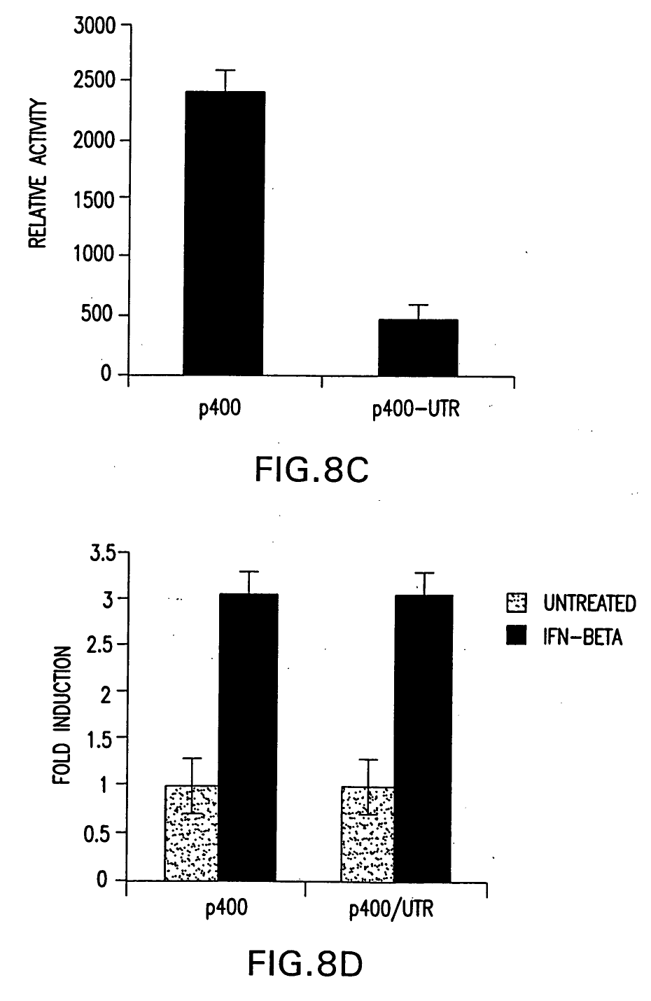

Since old-35 has an AU-rich 3′ UTR (FIG. 8A) it is possible that a post-transcriptional mechanism may also contribute to regulating old-35 mRNA levels in cells following IFN-β stimulation. To address this question old-35 mRNA stability was determined using an actinomycin D (AD) treatment protocol. Since AD inhibits RNA polymerase activity, mRNA synthesis is terminated and the decay of mRNA synthesized before AD treatment can be determined. Total RNA was collected at different time points and quantified by Northern blotting. Using this protocol, the half-life of old-35 mRNA in untreated and IFN-β treated HO-1 cells was determined. The half-life of old-35 in both untreated and IFN-β-treated HO-1 was ˜6 h (FIG. 8B). To confirm these results by a different method, the old-35 3′ UTR was cloned into the p400 construct and this construct and the unmodified p400 construct were transfected into HO-1 cells and luciferase activity was determined. These experiments revealed that even though the stability of the original old-35 promoter fragment mRNA was decreased upon cloning of the old-35 3′ UTR into the p400 plasmid (FIG. 8C), this modification did not alter stability following IFN-β treatment (FIG. 8D). These experiments suggest that post-transcriptional modifications of old-35 mRNA may not be major contributing factors in IFN-β upregulation of old-35 mRNA in HO-1 cells.

New protein synthesis is not required for induction of old-35 mRNA by IFN-β. To test whether old-35 expression requires newly synthesized proteins, the effect of inhibiting protein synthesis, using cycloheximide (CHX), on old-35 expression was determined. Treatment of HO-1 cells with IFN-β in the absence of CHX resulted in elevated old-35 expression. This upregulation was further potentiated when HO-1 cells were treated with IFN-β in the presence of CHX (FIG. 8E). These experiments suggest that IFN-β mediated upregulation of old-35 is not dependent on newly synthesized proteins and implicate old-35 as an early IFN response gene.

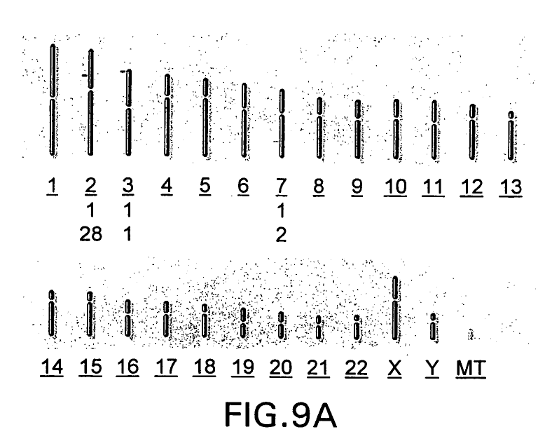

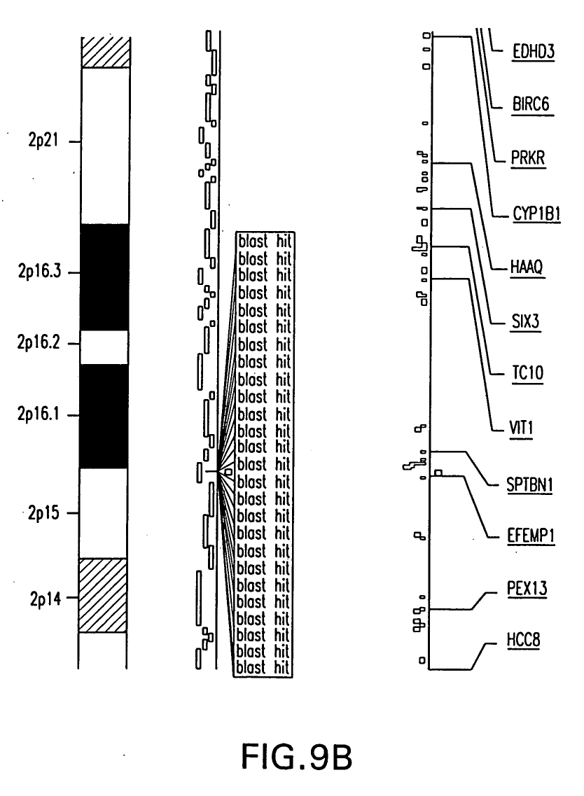

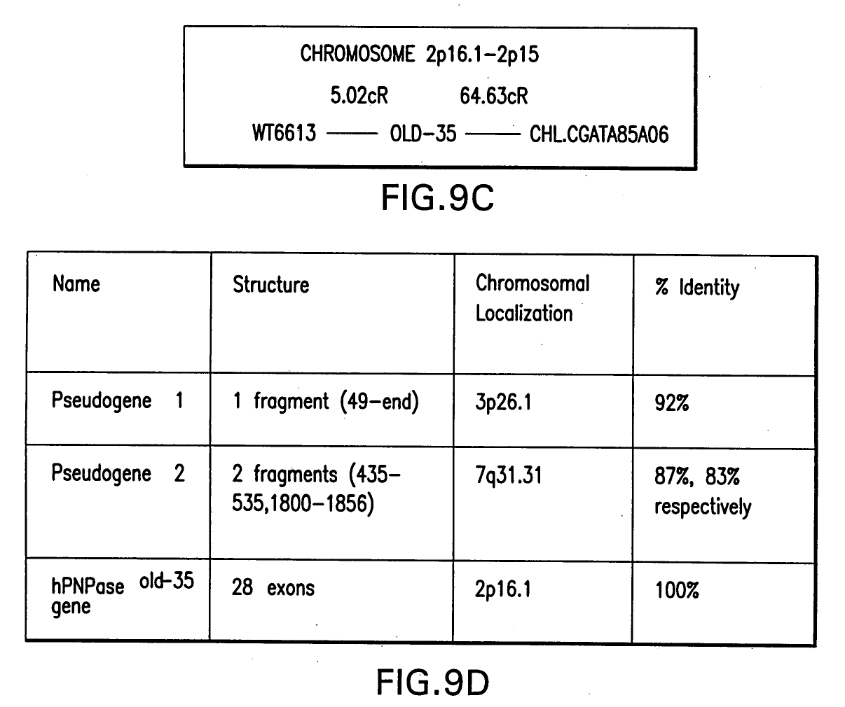

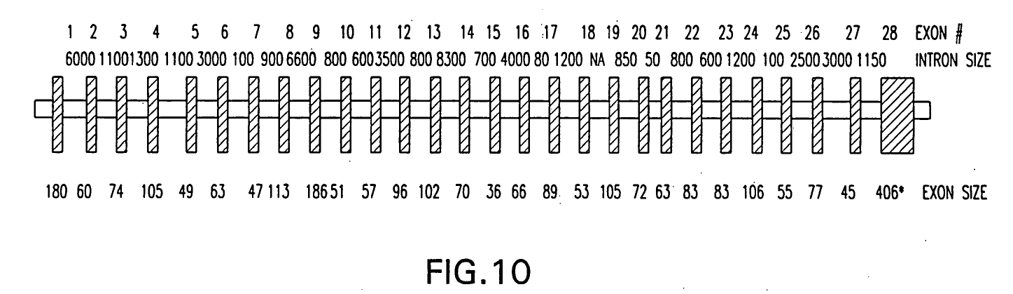

Genomic structure of old-35. To elucidate old-35 genomic structure, the NCBI database was screened using a BLAST search through the existing, partially sequenced, BAC clones. Using this bioinformatics approach we identified two BACs that contained old-35 sequences. The RP11-327M20 BAC clone contains an old-35 pseudogene that localizes to 3p26.1. This genomic fragment contains old-35 cDNA beginning from nucleotide 49 to the end of the cDNA and it is 92% homologous to old-35 cDNA (FIG. 9D). Using the Gene Jockey sequence analysis program, it was determined that this DNA does not have an open reading frame due to numerous mutations, thus it cannot encode OLD-35 protein. The second BAC clone, RP11-152O18, contains the old-35 gene since genomic sequences within this BAC clone exhibit 100% homology to the old-35 cDNA (FIG. 9D). The exon-intron junctions of old-35 were determined starting from the last exon and continuing in the 5′ direction. The exon-intron boundaries for exon 1 to exon 10 were determined from available sequence information in the database. The approximate sizes of the introns were determined by PCR using primers near the exon-intron boundaries (FIG. 10, FIG. 12). The complete genomic region of old-35 spans ˜54 kb and consists of 28 exons and 27 introns (FIG. 10). Exon sizes vary from 1960 bp (exon 28) to 36 bp (exon 15) with the last exon being the longest. Intron sizes range from 80 bp (intron 16) to over 8 kb (intron 13) with the average being ˜1 kb. FIG. 12 summarizes the locations of the exon-intron junctions as well as the sequences at the junctions. Almost all exon-intron junctions follow the consensus rule of the splice acceptor-AG/GT-splice donor for splicing.

Chromosomal localization of old-35. Two methods were used to determine chromosomal localization of the old-35 gene. Using Gene Bridge 4 (Research Genetics) we were able to determine that old-35 localizes to the second chromosome and it is positioned 5.02 cR from WI-6613 and 64.63 cR from CHLC.GATA85A06 markers (FIG. 9C). This result was confirmed by a BLAST search of the genomic database which localized old-35 to the border between 2p15-2p16.1 (FIGS. 9A and 9B). This search also identified two additional old-35 pseudogenes; the first localized to 3p26.1 (a highly mutated form of the old-35 cDNA), and the second to 7q31.31 (FIGS. 9A and 9B).

Forced expression of old-35 induces morphological changes and cell death. To better understand the functional role of old-35 during differentiation and senescence, the recombinant adenovirus (Ad) shown in FIG. 13A (Ad.old-35), in which transcription of the old-35 cDNA is driven by the CMV promoter, was constructed and employed to achieve forced expression of old-35. Because old-35 was originally cloned from HO-1 cells, the effects of forced expression of old-35 were initially examined in these cells.

As demonstrated in FIG. 13, transduction of HO-1 cells by Ad.old-35 or by an Ad vector lacking any expressible transgene (Ad.vec), at a multiplicity of infection (MOI) of 100 plaque-forming units (pfu) per cell, resulted in an inhibition of growth and eventually in cell death. During the process of dying, the Ad.old-35-transfected cells exhibited characteristic morphological changes in which the dying cells aggregated to form a clump. Similar morphological changes were also observed in HO-1 cells undergoing terminal differentiation following IFN-β and Mez treatment or if kept in culture for a prolonged period of time. These findings indicate that old-35 is directly involved in the molecular machinery controlling differentiation and senescence.

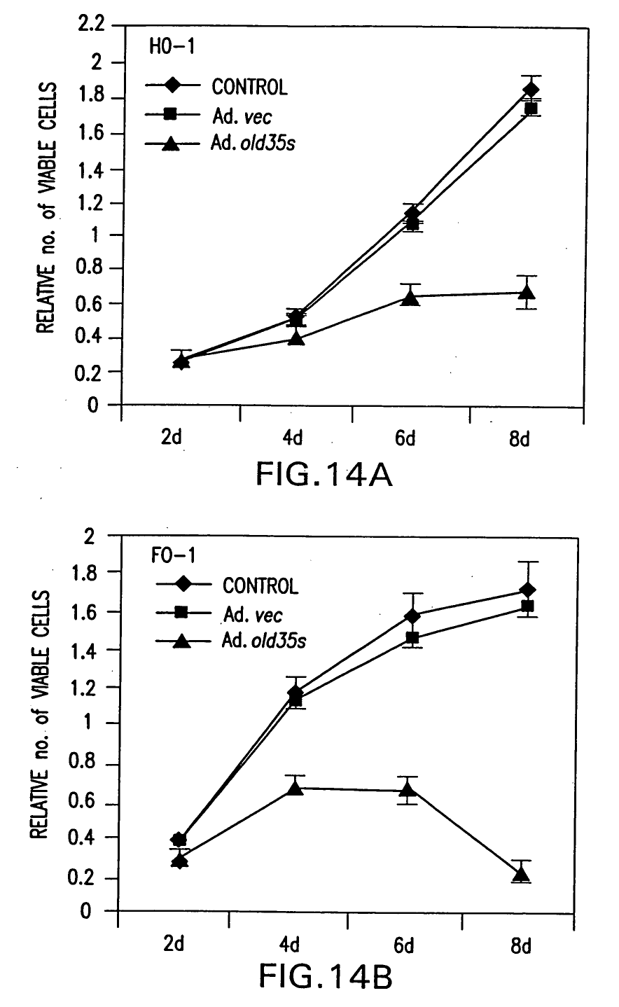

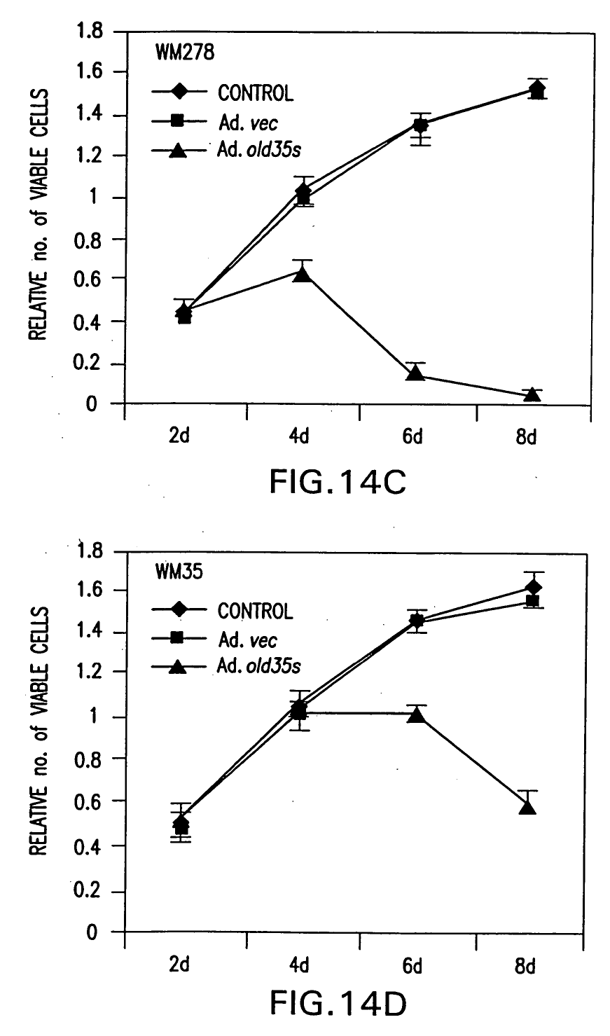

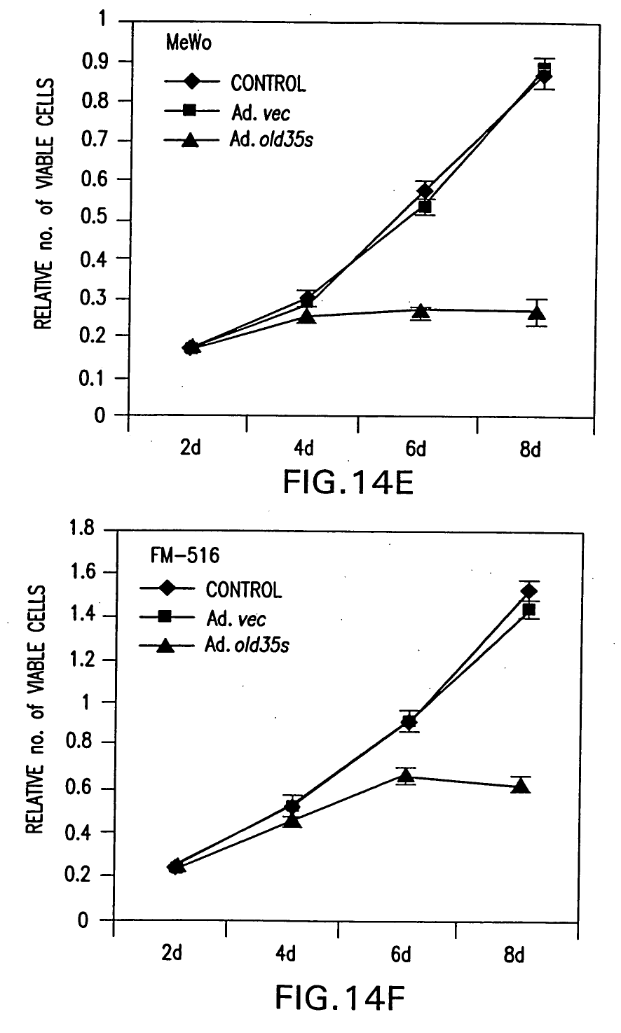

Additional studies, shown in FIG. 14, were performed to quantify these findings in HO-1 cells and to extend these findings to other cell populations including FO-1, WM278, WM35, and MeWo melanoma cells, and normal melanocytes immortalized with SV40 Tag (FM-516). The viability of all of these cell types, as determined using a standard MTT assay, was significantly reduced by exposure to Ad.old-35 at a MOI of 100 pfu/cell (FIG. 14). In addition, transduction by Ad.old-35 also inhibited growth of a diverse array of cancer cell lines, e.g. prostate, breast and colorectal carcinoma, glioblastoma multiforme, osteosarcoma (SaOS2), and fibrosarcoma (2fTGH), and also normal cells e.g. skin fibroblasts and primary fetal astrocytes. The growth inhibitory effect of Ad.old-35 was independent of p53 or Rb status of the cells.

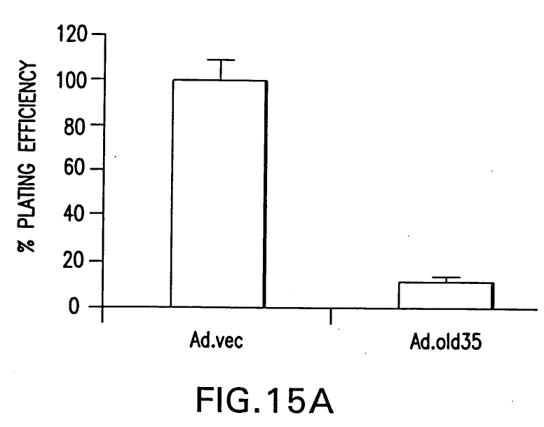

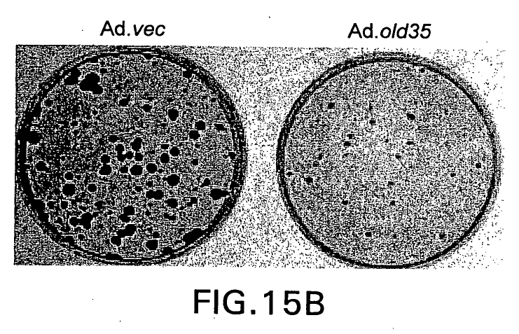

Forced expression of old-35 reduces plating efficiency, induces apoptosis and inhibits DNA synthesis in HO-1 cells. The inhibitory effects of old-35 expression on HO-1 cell growth were confirmed in studies of HO-1 colony formation. In these studies, HO-1 cells were transduced by Ad.old-35 or Ad.vec at a MOI of 100 pfu/cell, and after six hours of culture, the cells were trypsinized and 1000 cells were plated per 6-cm dish. The number of colonies formed was then determined after an additional three weeks of culture. As shown in FIG. 15, transduction by Ad.old-35 decreased the colony formation by ˜90% relative to HO-1 cells transduced by the control vector.

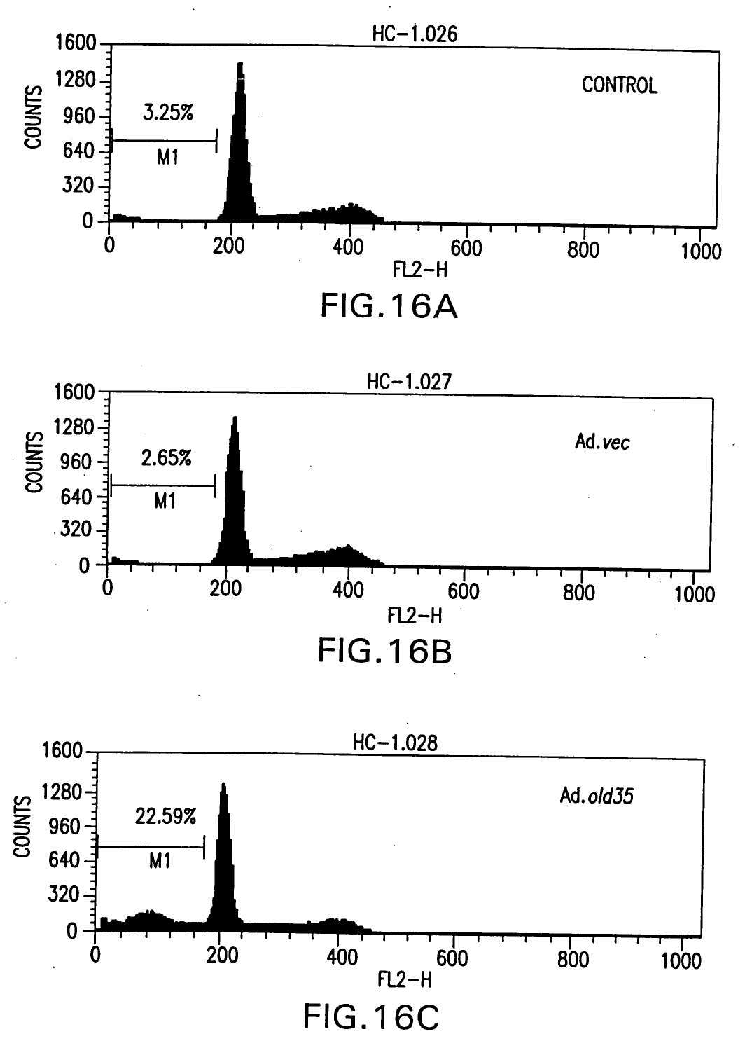

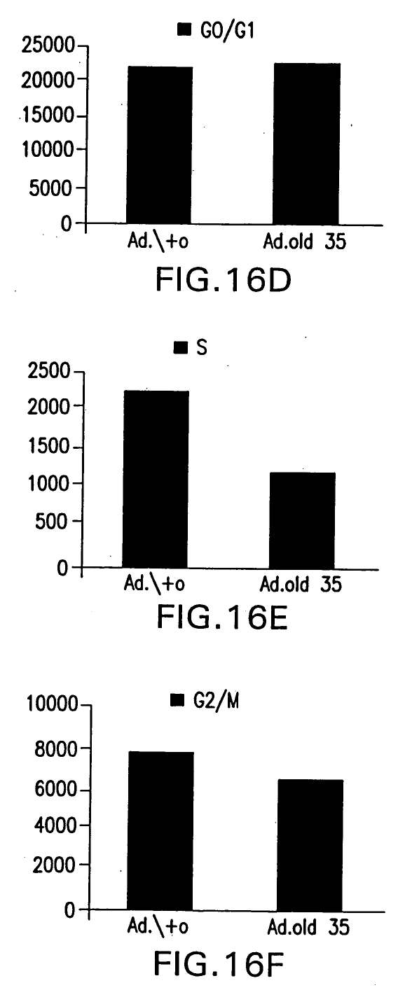

To understand the mechanism of Ad.old-35 mediated growth inhibition, cell cycle analysis by flow cytometry was carried out in HO-1 cells. As shown in FIG. 16A, a significant population (22.59%) of apoptotic cells could be detected at four days after transduction by Ad.old-35. In contrast, only a few (2.65% and 3.29%) apoptotic cells could be detected in non-transduced or control transduced (Ad.vec) cells, respectively. Additionally, as shown in FIG. 16B, the number of cells in S-phase of cell cycle was also significantly decreased in HO-1 cells transduced by Ad.old-35 as compared to control transduced cells, indicating that expression of old-35 also results in inhibition of DNA synthesis. The induction of apoptosis and decrease in the S-phase observed in Ad.old-35-transduced HO-1 cells was also detected in other cell lines that are susceptible to transduction by recombinant adenoviruses (data not shown). Terminally differentiating cells also show decreased S-phase and ultimately go into apoptosis and one of the markers of senescent cells is reduced S-phase. Thus, cells in which expression of old-35 is forced display many of the same features as cells in which terminal differentiation and senescence are occurring.

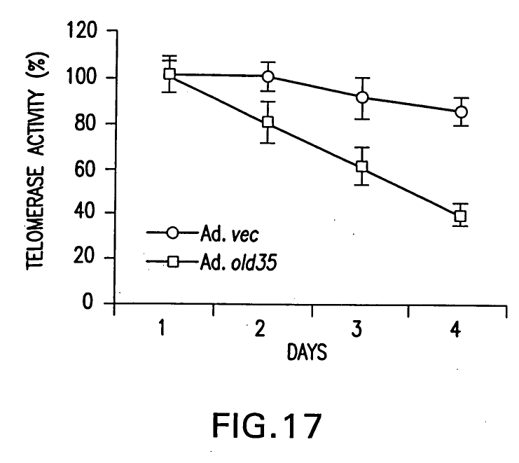

Forced expression of old-35 inhibits telomerase activity and alters the expression of pro- and anti-apoptotic proteins. Both terminal differentiation and senescence are associated with reduced telomerase activity. Therefore, telomerase activity was examined directly in cells transduced by Ad.old-35. As shown in FIG. 17, transduction by Ad.old-35, at a MOI of 100 pfu/cell, significantly reduced telomerase activity in HO-1 cells beginning from two days post-transduction.

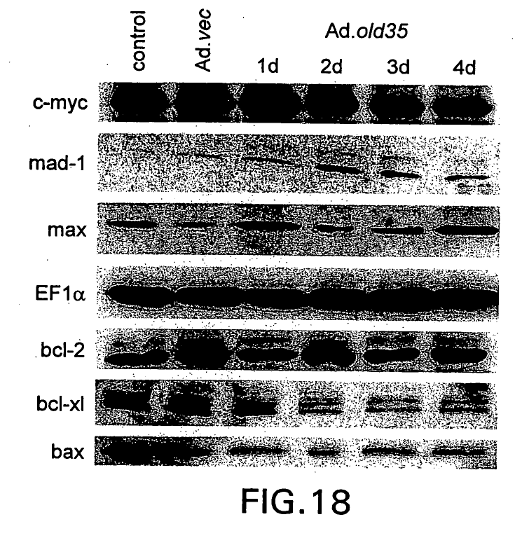

The transcription factor c-myc is an important regulator of hTERT (human telomerase reverse transcriptase), a protein that controls telomerase activity, and previous studies had shown that expression of c-myc decreases significantly in terminally differentiated HO-1 cells (Jiang et al, 1995, Oncogene 11:1179-1189). In the studies described herein, HO-1 cells were either non-transduced or transduced by Ad.vec or Ad.old-35 at a MOI of 100 pfu/cell. Whole cell extracts were prepared at the indicated time points. For non-transduced and Ad.vec-transduced cells, whole cell extracts were prepared at day 4 post-transduction. 30 μg of whole cell extract were electrophoresed and transferred to a nitrocellulose membrane. The expression levels of the indicated proteins were analyzed by Western blot analysis. As shown in FIG. 18, forced expression of old-35 in HO-1 cells by transduction with Ad.old-35 resulted in a significant decrease in c-myc expression, which began a two days post-transduction. This decrease in c-myc expression was correlated with the reduction in telomerase activity observed above, indicating a close relationship between decreased c-myc expression and telomerase activity downregulation.

In normal cells, c-myc heterodimerizes with max, and the myc/max heterodimer binds to specific DNA sequences to facilitate progression of the cell cycle from G1 to S-phase. Under certain circumstances, including differentiation, the expression of c-myc goes down and the expression of mad-1 goes up. The mad-1/max heterodimer inhibits progression of the cell cycle resulting in growth arrest. A similar phenomenon was observed in HO-1 cells transduced by Ad.old-35, where c-myc expression went down, mad-1 expression gradually went up, and the expression of max was unchanged (FIG. 18).

As discussed above, forced expression of old-35 induced apoptosis in HO-1 cells. To further explore this phenomenon, the levels of the anti-apoptotic bcl-2 and bcl-xl proteins and the pro-apoptotic bax protein were also examined by Western analysis, performed as described above for c-myc. In these studies, the results of which are also shown in FIG. 18, transduction of HO-1 cells by Ad.old-35 significantly reduced intracellular levels of bcl-xl, while the levels of bcl-2 and bax remained unchanged.

c-myc expression plays an important role in old-35-mediated cell death. To better understand the role of c-myc in Ad.old-35 mediated growth inhibition, HO-1 cells were transiently transfected with a c-myc-expressing plasmid and, after 36 hrs in culture, the transfected cells were transduced by Ad.old-35 at a MOI of 50 or 100 pfu/cell. At 6 hrs post-transduction, the cells were trypsinized, counted and plated at a density of 1000 cell per 6-cm dish. The cells were allowed to form colonies for 3 weeks and the number of colonies were then counted.

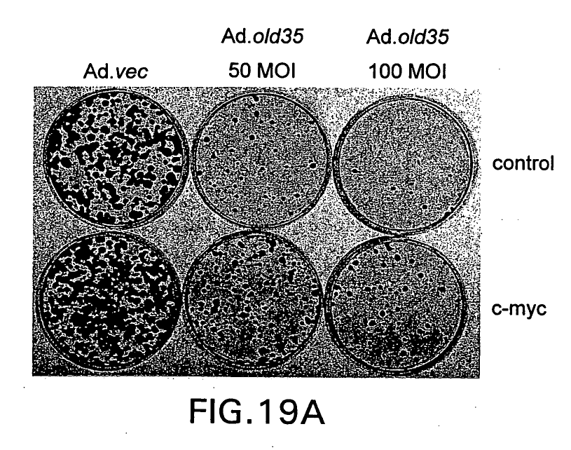

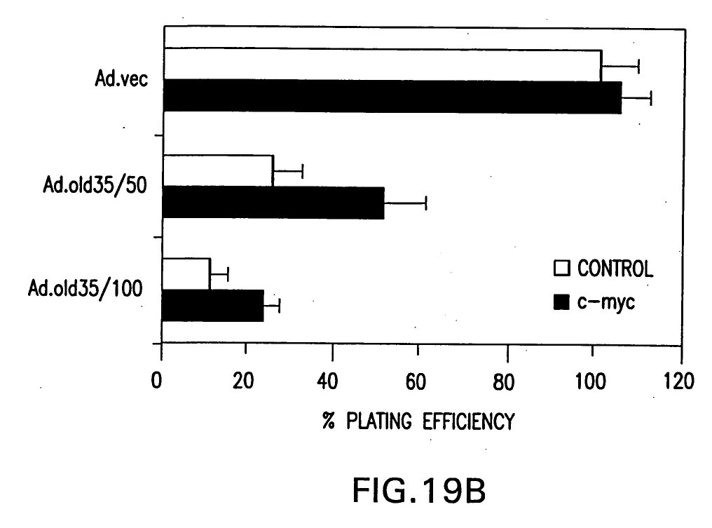

As shown in FIG. 19, transduction by Ad.old-35 at a MOI of 50 pfu/cell reduced the colony number by ˜75% while transduction by Ad.old-35 at 100 pfu/cell reduced colony formation by 90%. Overexpression of c-myc provided partial but significant protection against Ad.old-35-mediated killing. This finding indicates that downregulation of c-myc plays an important role in Ad.old-35-mediated cell death.

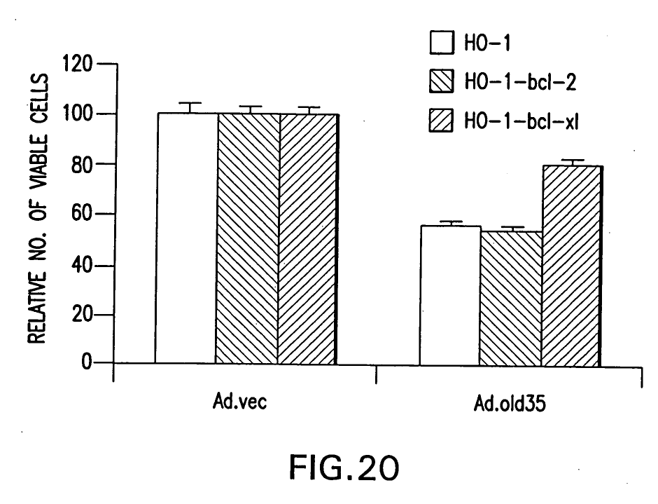

Overexpression of bcl-xl protects against the apoptotic effects of old-35 expression. To confirm a protective role for bcl-xl in the old-35-mediated toxicity of HO-1 cells, HO-1 cells stably transformed to overexpress either bcl-2 or bcl-xl were transduced by either Ad.vec or Ad.old-35. Seven days after transduction, cell viability was measured by standard MTT assay. As shown in FIG. 20, overexpression of bcl-xl partially protected against Ad.old-35-mediated killing, while bcl-2 overexpression exerted no protective effects. Without being bound by any particular theory, these findings indicate that Ad.old-35-mediated apoptosis goes through a mitochondrial pathway involving bcl-xl.

Forced expression of old-35 alters the expression of cyclin-dependent kinase inhibitors but not p53. Senescent cells arrest irreversibly in G1 phase of the cell cycle resulting in a profound decrease in DNA synthesis (S-phase). These cells also resist apoptosis, while terminally differentiated cells ultimately die by apoptosis. As shown above in FIG. 16, transduction of HO-1 cells by Ad.old-35 did not induce any G1-arrest, but did reduce S-phase.

Several factors control progression of the cell cycle beyond G1 phase. One of these is c-myc, which facilitates cell cycle progression and is down-regulated by expression of old-35 (FIG. 18). Another group of proteins that prevent cell cycle progression are the cyclin-dependent kinase inhibitors (CDKIs). Growth arrest in senescent cells is associated with overexpression of one or more of the CDKIs, such as p15, p16, p21 and p27. In some models of senescence, overexpression of one of the CDKIs is associated with downregulation of others.

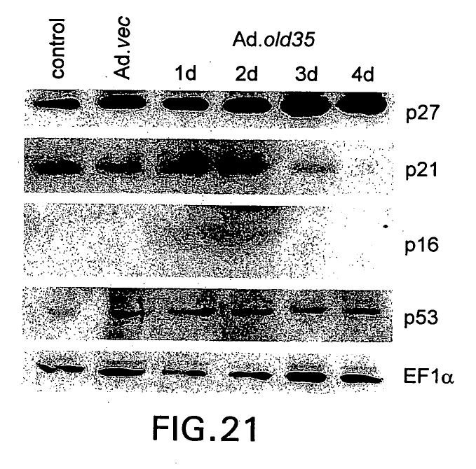

To examine the effects of old-35 expression on CDKIs, HO-1 cells were either non-transduced or transduced with Ad.vec or Ad.old-35 at a MOI of 100 pfu/cell. Whole cell extracts were prepared at four days post-transduction for the non-transduced or Ad.vec-transduced cells, or at the time points indicated in FIG. 21 for the cells transduced by Ad.old-35. 30 μg of whole cell extract were electrophoresed and transferred to a nitrocellulose membrane. The expressions of the indicated proteins were then analyzed by Western blot analysis.

As shown in FIG. 21, forced expression of old-35 resulted in a significant increase in the expression of p27, and a significant decrease in the expression of p21. The expression of p16 could not be detected in HO-1 cells, which might be due to the fact that a majority of the melanomas have genetic alteration in p16. The expression of another important gene that controls cell growth, cell cycle and apoptosis, p53, appeared unaltered following forced expression of old-35. The expression patterns of CDKIs and p53 observed in HO-1 cells following forced expression of old-35 are similar to those observed in premature senescence models in hepatocytes by iron-chelation or in fibroblasts by inhibition of P13 kinase pathway.

Forced expression of old-35 increases the phosphorylation of PKR and eIF2α Old-35 is an interferon-inducible gene and it is primarily a 3′-5′ RNA exonuclease. Thus, it is likely that old-35 is a part of the interferon-regulated RNA processing machinery. The growth inhibitory effect of interferons are partially mediated by double-stranded RNA-dependent protein kinase R (PKR), which phosphorylates eukaryotic initiation factor 2α (eIF2α), thereby resulting in translational inhibition and growth arrest. Phosphorylation of eIF2α also induces the expression of growth arrest and DNA damage-inducible gene (GADD153), which induces growth arrest and apoptosis.

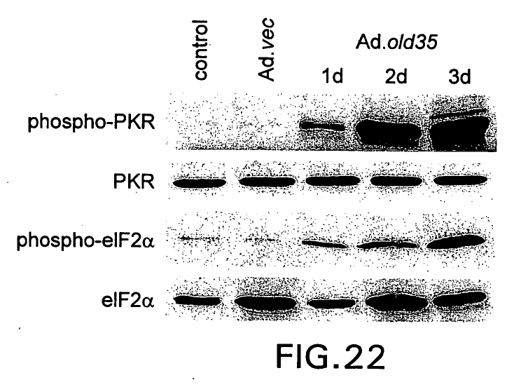

To determine if any relationship existed between Ad.old-35-mediated growth inhibition and the PKR pathway, HO-1 cells were either non-transduced or transduced with Ad.vec or Ad.old-35 at a MOI of 100 pfu/cell. Whole cell extracts were prepared at three days post-transduction for the non-transduced or Ad.vec-transduced cells, or at the time points indicated in FIG. 22 for the cells transduced by Ad.old-35. 30 μg of whole cell extract were electrophoresed and transferred to a nitrocellulose membrane. The expressions of the indicated proteins were then analyzed by Western blot analysis. As shown in FIG. 22, transduction of HO-1 cells by Ad.old-35 resulted in phosphorylation of PKR as well as eIF2α.

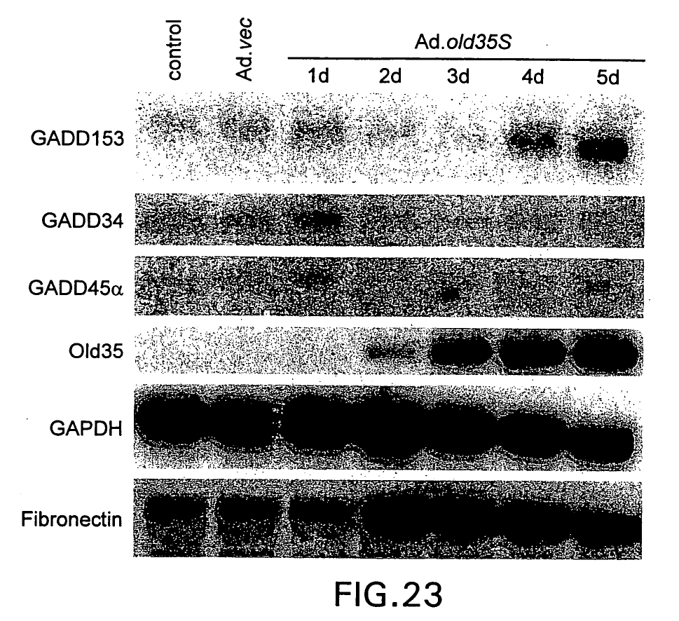

As shown in FIG. 23, the expression of GADD153 was also subsequently induced in the Ad.old-35-transduced cells. In these studies, HO-1 cells were either non-transduced or transduced with Ad.vec or Ad.old-35 at a MOI of 100 pfu/cell. Whole cell extracts were prepared at five days post-transduction for the non-transduced or Ad.vec-transduced cells, or at the time points indicated in FIG. 23 for the cells transduced by Ad.old-35. 30 μg of whole cell extract were electrophoresed and transferred to a nitrocellulose membrane. The expressions of the indicated proteins were then analyzed by Western blot analysis. Transduction by Ad.old-35 did not change the expression of other GADD family genes, like GADD45 and GADD34, indicating the specificity of this pathway. Although old-35 is an RNA exonuclease, the lack of effect of forced expression of old-35 did not cause generalized degradation of RNA. Thus, old-35 exerts substrate specificity.

Forced expression of old-35 increases the expression of fibronectin. Because the cell death associated with the forced overexpression of old-35 was associated with cell clumping, the effects of old-35 expression on the expression of the fibronectin protein were examined. In these studies, the results of which are also shown in FIG. 23, the expression of fibronectin increased significantly in the cells transduced by Ad.old-35 beginning approximately two days post-transduction. Fibronectin is also upregulated both during both terminal differentiation and senescence, further suggesting the similarity between old-35 expression and these two cellular processes.

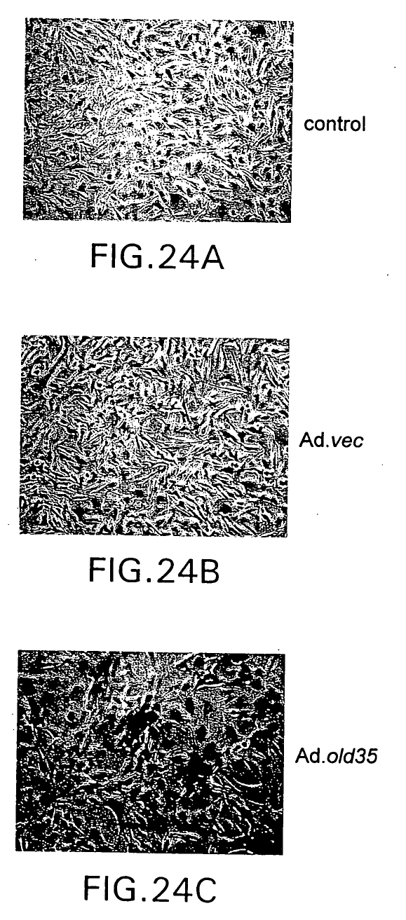

Forced expression of old-35 induces senescence-associated β-galactosidase activity. Because of the similarity between the morphological and biochemical changes induced by old-35 overexpression and senescence, the effects of forced overexpression of old-35 on senescence-associated β-galactosidase (SA-β-GAL) activity were observed. In these studies, HO-1 cells were either non-transduced or transduced by either Ad.vec or Ad.old-35 at a MOI of 100 pfu/cell. SA-β-GAL activity was then measured at four days post-transduction. As shown in FIG. 24, transduction of HO-1 cells by Ad.old-35 significantly induced SA-β-GAL activity, while non-transduced or Ad.vec-transduced HO-1 cells did not show any SA-13-GAL positive cells. Together, the findings summarized above indicate that expression of old-35 induces profound morphological, biochemical and molecular changes in HO-1 cells, many of which correlate with the features of terminal differentiation and senescence.

6.3 Discussion

IFNs represent physiologically important cytokines with potent growth and immune regulatory properties (Fisher and Grant, 1985, Pharmacol. Ther. 27: 143-166; Greiner et al., 1985, Pharmacol. Ther. 31: 209-236; Pestka et al., 1987, Annu. Rev. Biochem. 56: 727-777; Stark et al., 1998, Annu. Rev. Biochem. 67: 227-264). Their effects are mediated primarily through the activation of transcription of many downstream effector genes (Stark et al., 1998, Annu. Rev. Biochem. 67: 227-264; Schindler and Darnell, 1995, Annu. Rev. Biochem. 64: 621-651; Der et al., 1998, Proc. Natl. Acad. Sci USA 95: 15623-15628). IFNs bind to the IFN receptor and activate the JAK/STAT signaling cascade resulting in the upregulation of many IFN stimulated genes (ISGs) (Stark et al., 1998, Annu. Rev. Biochem. 67: 227-264; Schindler and Darnell, 1995, Annu. Rev. Biochem. 64: 621-651; Der et al., 1998, Proc. Natl. Acad. Sci USA 95: 15623-15628; Colamonici et al., 1994, J. Biol. Chem. 269:3518-3522). Understanding the regulation and function of the ISGs is germane since it will contribute to our knowledge of IFN function. Here we describe the cloning, expression, genomic structure and regulation of old-35, a Type 1 IFN gene induced by IFN-α and IFN-β in melanoma and other cell types. Melanoma cells express two old-35 mRNA species, a predominant ˜2.6-kb and an ˜4.3-kb variant, that exhibit 100% homology in their coding region. These mRNA species differ, however, in the length of their 3′ UTRs, possibly resulting from differential polyadenylation. Expression of old-35 is induced as early as 3 h by as little as 1 unit/ml of IFN-β in HO-1 melanoma cells suggesting that old-35 is an early response gene and that its expression depends on the JAK/STAT signaling cascade. Since double stranded RNA also stimulates old-35 expression, it is possible that old-35 may be involved in cellular response to viral infection mediated by the Type 1 IFNs. The fact that old-35 can be induced by IFNs in most cell types, including those with a wild-type or a mutant p53 genotype, suggests that induction of old-35 by IFNs may represent a general cellular response to these cytokines. It is worth noting that old-35 mRNA expression as monitored by Northern blotting in HO-1, FO-1 and HeLa cells corresponds with old-35 promoter activity. This indicates that differences in old-35 expression in these cells are most likely a consequence of differential transcription of the old-35 gene.

Defining the biological consequence of old-35 expression may provide important insights into cellular responses to Type 1 IFN. To obtain clues into the mechanism by which old-35 expression is enhanced by Type 1 IFN treatment, the promoter region of the old-35 gene was isolated and characterized. Sequence analysis of the old-35 promoter region identified several IFN-related binding sites: including two IRF-1 binding sites, one GAS element and one ISRE element. The ISRE element, which consists of the sequence GAAAN(N)GAAA (SEQ ID NO:117), binds IFN-stimulated gene factor 3 (ISGF3), a complex composed of STAT1/STAT2 heterodimer and the IFN regulatory factor (IRF) p48 (48-50). Mutation of this ISRE element in the old-35 promoter eliminates binding of the ISGF3 complex and inhibits IFN-mediated old-35 induction (FIGS. 6C and 6D). A similar point mutation in the ISRE elements in other ISG promoters also renders them unresponsive to IFNs (Brakebusch et al., 1999, Genomics 57: 268-278; Lefebvre et al., 2001, J. Biol. Chem. 276: 6133-6139). The IRF-1 element has also been shown to bind to ISRE-like sequences, suggesting that IRF-1 and ISGF3 might regulate an overlapping set of genes regulated by ISG promoters (Levy et al., 1986, Proc. Natl. Acad. Sci. USA 83: 8929-8933; Friedman et al., 1984, Cell 38: 745-755; Li et al., 1998, J. Interferon Cytokine Res. 18: 947-952). In a manner analogous to the IRFs, STATs can also bind DNA. Stat1 homodimer, also called IFN-activation factor (GAF), binds the IFN-activation site (GAS), which contains the AANNNNNTT consensus sequence (SEQ ID NO:118; Schindler and Darnell, 1995, Annu. Rev. Biochem. 64: 621-651). Deletion of either the IRF-1 or GAS element does not affect the IFN-β inducible activity of the old-35 promoter in HO-1, FO-1, HeLa or 2fTGH cells. The IFN responsive 2′-5′ oligoadenylate synthetase (OAS) promoter contains the same arrangement of IRF-1 and ISRE as does the old-35 promoter (Wang and Floyd-Smith, 1998, Gene 222: 83-90). However, all elements in the OAS promoter are necessary to achieve maximal activity upon IFN stimulation. The reason for the differential IFN-dependency of the old-35 and OAS promoters is not known, but it may reflect the cellular context in which these promoters were tested. Alternatively, subtle differences in the structures of these promoters, or the presence of additional binding elements, may define differences in IFN responses. Other factors such as C/EBPβ may act as a co-activator in regulating IFN responsiveness (Roy et al., 2000, J. Biol. Chem. 275: 12626-12632). Moreover, different combinations of ISRE and GAS elements in the promoters of diverse IFN stimulated genes may also help orchestrate the complex regulation of gene expression changes induced by the IFNs. JAK/STAT signaling pathways can induce transcription of specific genes in the absence of new protein synthesis (Lehtonen et al., 1997, J. Immunol. 159: 794-803). However, the inability of IFN to induce specific ISGs in cells pretreated with a protein synthesis blocker, such as cycloheximide, suggests that other inducible factors may contribute to IFN-activation of these genes (Gongora et al., 2000, Nucleic Acids Res. 28 2333-2341). Old-35 upregulation by IFN-β does not depend on new protein synthesis, suggesting that old-35 is an early response gene directly induced by the JAK/STAT signaling cascade. It is demonstrated that the JAK/STAT pathway is essential for old-35 activation since old-35 expression is abolished in the fibrosarcoma JAK/STAT mutants. Additionally, incubation of HO-1 cells with CHX in the absence of IFN-β treatment results in induction of old-35 mRNA suggesting that either old-35 transcription or old-35 mRNA stability may be suppressed in HO-1 cells. However, since the half-life of old-35 is not altered in HO-1 cells following IFN-β treatment, it appears more likely that proteins affecting old-35 transcription limit expression of this gene in the uninduced state.

Gene expression is regulated not only by a transcriptional mechanism but also by post-transcriptional mechanisms, which regulate mRNA stability (Spicher et al., 1998, Mol. Cell. Biol. 18: 7371-7382). In mammals, post-transcriptional regulation appears to be important in cells responding to environmental stress, proliferation and differentiation (Sierra and Zapata, 1994, Mol. Biol. Rep. 19: 211-220). Currently, three classes of destabilizing elements have been identified: AUUUA-lacking elements and AUUUA-containing elements grouped into those with scattered AUUUA motifs (such as proto-oncogenes) and those with overlapping AUUUA motifs (such as growth factors) (Chen and Shyu, 1995, Trends Biochem. Sci. 20: 465-470). Exchange of a 3′ UTR containing ARE for a 3′ UTR of a stable message, such as β-globin, targets this very stable mRNA for rapid degradation (Shaw and Kamen, 1986, Cell 46: 659-667). In accordance with previous reports, insertion of old-35 UTR in the p400 construct resulted in lower luciferase activity suggesting a destabilizing effect of this UTR in HO-1 cells (FIG. 8C).

Additionally the results showed that that differential stability of old-35 does not play a role in higher levels of old-35 mRNA observed following IFN-β treatment (FIG. 8D). However, it is still possible that 3′ UTR may induce a stabilizing effect following other treatments or in specific cellular states, such as terminal differentiation or senescence, thereby contributing to elevated old-35 expression.

Defining the genomic structure of a novel gene can prove valuable in addressing questions relevant to gene function, since these findings can provide information about various splice variants, polyadenylation differences and expression controlling elements in the promoter. Genomic analysis of the old-35 gene revealed that this gene is positioned on 28 exons that span ˜54-kb (FIG. 10). Analysis of old-35 genomic structure assisted in determining that the difference between the two old-35 mRNA variants was due to differential polyadenylation and not differential splicing, since there were no introns separating the shorter ˜2.6-kb and longer ˜4.3-kb variant. In addition to the old-35 gene, two old-35 pseudogenes were identified in the human genome. The old-35 gene maps to 2p16, with a region 2p15-p21 frequently involved in cytogenetic alterations in human cancers (Kirschner et al., 1999, Genomics 62: 21-33). Moreover, this region contains genes potentially involved in multiple genetic disorders, including Type I hereditary nonpolyposis colorectal cancer, familial male precocious puberty, Carney complex, Doyne's honeycomb retinal dystrophy and DYX-3 a form of familial dyslexia (Shaw and Kamen, 1986, Cell 46: 659-667; Kirschner et al., 1999, Genomics 62: 21-33; Stratakis, 2001, Ann. Endocrinol. (Paris) 62: 180-184).

The OLD-35 protein exhibits high homology to a 3′-5′ RNA exonuclease, polyribonucleotide phosphorylase (PNPase), an important enzyme implicated in the degradation of bacterial messenger RNAs (Portier et al., 1981, Mol. Gen. Genet. 183: 298-305). This enzyme has also been found in plants where it functions in processing of plastid AU-rich 3′ UTR during chlororoplast differentiation (Hayes et al., 1996, EMBO J. 15: 1132-1141). It is possible that throughout evolution, starting from a simple degradation process, PNPases have been recruited to degrade more specific mRNAs during processes such as differentiation and defense against viral infection.

### 7. EXAMPLE

### Downregulation of Myc as a Potential Target for Growth Arrest Induced by Old-35 in Human Melanoma Cells

Cell Lines and Cell Viability Assays. Normal immortal human melanocyte (FM516-SV; FM516), WM35 early radial growth phase (RGP) and WM278 vertical growth phase (VGP) primary human melanomas, and HO-1, FO-1 and MeWo metastatic melanoma cell lines and HEK-293 cells were cultured as described in Lebedeva et al., 2002, Oncogene 21: 708-718. HO-1-pREP4 and HO-1-hPNPaseold-35AS cell lines were generated by stable transfection of HO-1 cells with pREP4 (HO-1-pREP4) or antisense hPNPase^{old-35} expressing pREP4 (HO-1-hPNPase^{old-35}AS), respectively (old-35 is also referred to in this section as human polynucleotide phosphorylase,^{old-35}, or hPNPase^{old-35}, with the corresponding protein designated hPNPase^{OLD-35}) and selection with hygromycin. HO-1-Bcl-2 and HO-1-Bcl-xL cell lines were produced by stable transfection of HO-1 cells with Bcl-2 and Bcl-xL expression plasmids (kindly provided by Dr. John C. Reed) and selection with G418. Cell growth and viable cell numbers were monitored by 3-(4,5-dimethylthiazol-2-yl)-2,5-diphenyl tetrazolium bromide (MTT) staining as described in Lebedeva et al., 2002, Oncogene 21: 708-718. Cultures were incubated with interferon-beta (2000 units/ml) and mezerein (10 ng/ml) for 5 days prior to assaying for cell viability.

Virus Construction and Infection Protocol. The construction and purification of hPNPase^{old-35} expressing replication-defective Ad.hPNPase^{old-35} were described in Leszczyniecka et al., 2002, Proc Natl Acad Sci USA 99: 16636-16641 and Valerie, 1999, Biopharmaceutical Drug Design and Development (Wu-Pong and Rojanasakul, Eds.), Humana press, Totowa, N.J. A similar method was employed to generate an antisense hPNPaseold-35 expressing replication-defective adenovirus (Ad.hPNPase^{old-35}AS). The empty adenoviral vector (Ad.vec) was used as a control. Viral infections were performed as described in Lebedeva et al., 2002, Oncogene 21: 708-718.

Plasmid Construction, Transfection and Colony Formation Assays. 3′-HA-tagged hPNPase^{old-35} was created by PCR using the primers, sense: GCT AGC ATG GCG GCC TGC AGG TAC and antisense: GGA TCC TCA AGC GTA ATC TGG AAC ATC GTA TGG GTA CTG AGA ATT AGA TGA TGA. The authenticity of the amplified product was verified by sequencing and it was cloned into the NheI/BamHI sites of pcDNA3.1 (Invitrogen) to generate hPNPase^{old-35}-HA. hPNPase^{old-35}AS was generated by ligating hPNPase^{old-35} in an antisense orientation into BamHI/NotI sites of pREP4 (Invitrogen). The c-myc expression plasmid [p290-myc (Fisher et al., 1985, J Interferon Res 5: 11-22; Graham, et al., 1991, Cancer Immunol Immunother 32: 382-390)] was provided by Dr. Riccardo Dalla-Favera. HO-1 cells were plated at a density of 3×10⁵ cells per 6-cm dish and 24 h later were transfected with 5 μg of either empty vector or p290-myc (Fisher et al., 1985, J Interferon Res 5: 11-22; Graham, et al., 1991, Cancer Immunol Immunother 32: 382-390) using Superfect® (Qiagen, Hilden, Germany) transfection reagent according to the manufacturer's protocol. After 36 h, the cells were infected with Ad.hPNPase^{old-35} at an m.o.i. of 50 or 100 pfu/cell, 6 h later, the cells were trypsinized, counted and 10³ cells were plated in 6 cm dishes. Colonies were counted after 3 weeks. Colony formation assays using hPNPase^{old-35}-HA in HO-1 cells were performed as described (Kang, et al., 2002, Proc Natl Acad Sci USA 99: 637-642).

RNA Isolation and Northern Blot Analysis. Total RNA was extracted from the cells using Qiagen RNeasy mini kit (Qiagen) according to the manufacturer's protocol and Northern blotting was performed as described in Sarkar, et al., 2002, Proc Natl Acad Sci USA 99: 10054-10059. The cDNA probes used were a 400-bp fragment from human c-myc, a 500-bp fragment from hPNPase^{old-35}, a 500-bp fragment from human GADD34, full-length human c-jun and full-length human GAPDH.

In-Vitro Translation and In Vitro mRNA Degradation Assays. In vitro translation was performed using the TNT coupled Reticulocyte Lysate Systems (Promega, Madison, Wis.) using the plasmids pcDNA3.1 as a control, GADD153 expression plasmid and hPNPase^{old-35}-HA according to the manufacturer's protocol. Five μg of total RNA from HO-1 cells were incubated with 5 μl of each in-vitro translated protein at 37° C. from 0.5 to 3 h. The RNA was repurified using the Qiagen RNeasy mini kit (Qiagen) and Northern blotting was performed (Sarkar, et al., 2002, Proc Natl Acad Sci USA 99:10054-10059).

Western Blot Analysis. Western blotting was performed as described in Sarkar, et al., 2002, Proc Natl Acad Sci USA 99: 10054-10059. Briefly, cells were harvested in RIPA buffer containing protease inhibitor cocktail (Roche, Mannheim, Germany), 1 mM Na₃VO₄ and 50 mM NaF and centrifuged at 12,000 rpm for 10 min at 4° C. The supernatant was used as total cell lysate. Thirty μg of total cell lysate were used for SDS-PAGE and transferred to a nitrocellulose membrane. The primary antibodies included: from Santa Cruz Biotechnology, Santa Cruz, Calif.: Myc (1:200; mouse monoclonal), Max (1:200; rabbit polyclonal), Mad1 (1:200; rabbit polyclonal), p16 (1:200; rabbit polyclonal), p21 (1:200; rabbit polyclonal), p27 (1:200; rabbit polyclonal), p53 (1:200; mouse monoclonal), cyclin E (1:200, rabbit polyclonal), E2F1 (1:200, rabbit polyclonal); from BD biosciences: Rb (1:500, mouse monoclonal), cyclin A (1:500, mouse monoclonal); Bcl-2, Bcl-xL and Bax (1:1000; rabbit polyclonal; kindly provided by Dr. John C. Reed); anti-HA (1:3000; mouse monoclonal; Covance Research Products, Inc, Berkeley, Calif.) and EF1 alpha (1:1000; mouse monoclonal; Upstate Biotechnology, Waltham, Mass.).

³H-thymidine Incorporation Assay. HO-1 cells were plated at a density of 5×10⁴ cells in each well of a 12-well plate. The next day the cells were infected with Ad.hPNPaseold-35 at an m.o.i. of 25 or 50 pfu/cell. After 4 days the cells were incubated with 10 μCi/ml 3H-thymidine for 12 hours. The cells were washed with PBS and incubated with 2 ml of ice-cold 10% trichloroacetic acid (TCA) at 4° C. for 30 min. TCA precipitated materials were collected by centrifugation and solubilized with 1 ml of 2% SDS and 100 microliter aliquots were counted in a liquid scintillation counter.

Cell Cycle Analysis. Cells were harvested, washed in PBS and fixed overnight at −20° C. in 70% ethanol. The cells were treated with RNase A (1 mg/ml) at 37° C. for 30 min and then with propidium iodide (50 μg/ml). Cell cycle was analyzed using a FACScan flow cytometer and data were analyzed using CellQuest software (Becton Dickinson, San Jose, Calif.).

Telomerase assay. HO-1 cells were infected with either Ad.vec or Ad.hPNPase^{old-35} for 1 to 4 days or untreated or treated with fibroblast interferon (IFN-beta, 2000 units/ml) plus mezerein (MEZ, 10 ng/ml) for 1 to 4 days and telomerase assays were performed as described in Wood, et al., 2001, Oncogene 20: 278-288. Briefly, protein concentrations of cell extracts were determined and equal amounts of protein were used for the elongation process in which telomerase added telomeric repeats (TTAGGG) to the 3′-end of the biotin-labeled primer. These elongation products were amplified by PCR and the PCR products were denatured, hybridized to digoxigenin-labeled detection probes, specific for the telomeric repeats. The resulting products were immobilized via the biotin label to a streptavidin-coated microtiter plate. Immobilized amplicons were detected with an antibody against digoxigenin that is conjugated to horseradish peroxidase and the sensitive peroxidase substrate TMB. The telomerase activity was quantified by measuring the absorbance of the samples at 450 nm (with a reference wavelength of 690 nm) using a microtiter plate reader.

Statistical analysis. Statistical analysis was performed using one-way analysis of variance (ANOVA), followed by Fisher's protected least significant difference analysis.

Results: Previous studies demonstrated that infection with Ad.hPNPase^{old-35} inhibited colony formation in HO-1 melanoma cells (Leszczyniecka, et al., 2002, Proc Natl Acad Sci USA 99: 16636-16641). The present studies were conducted to comprehend the molecular mechanism underlying the growth arresting property of Ad.hPNPase^{old-35}. Different melanoma cell lines and SV40 T-Ag immortalized primary human melanocytes (FM-516-SV) were infected with Ad.hPNPase^{old-35} and the growth of the cells was monitored by standard MTT assays. As shown in FIG. 14, infection with Ad.hPNPase^{old-35} resulted in significant growth inhibition in all of the cells. The growth inhibitory effect became significant from 4 days post-infection and in certain cell lines (WM278 and MeWo) infection with Ad.hPNPase^{old-35} completely inhibited cell growth. In addition, Ad.hPNPase^{old-35} infection inhibited the growth of other cell types, including breast, prostate, colon and pancreatic carcinomas, glioblastoma multiforme, fibrosarcoma and osteosarcoma irrespective of their p53 or Rb status.

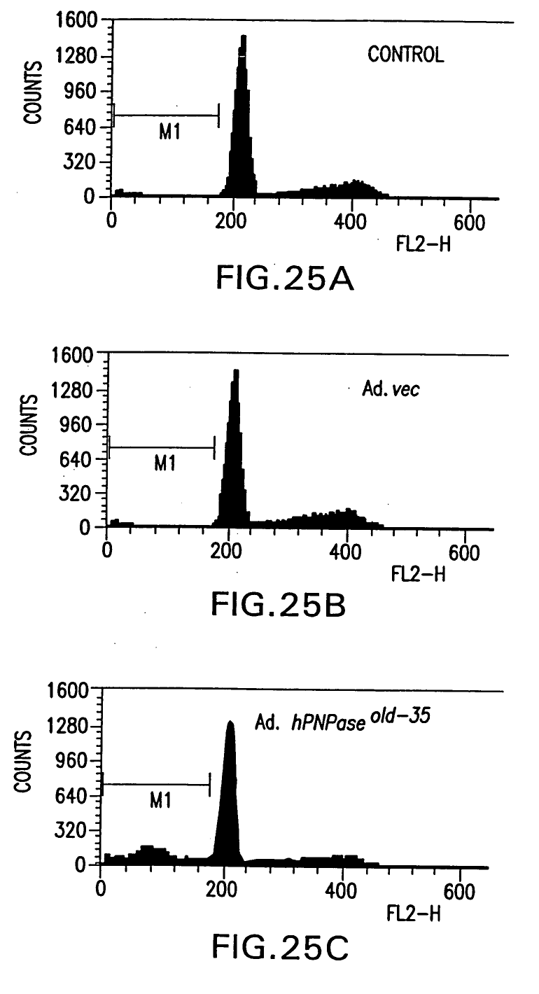

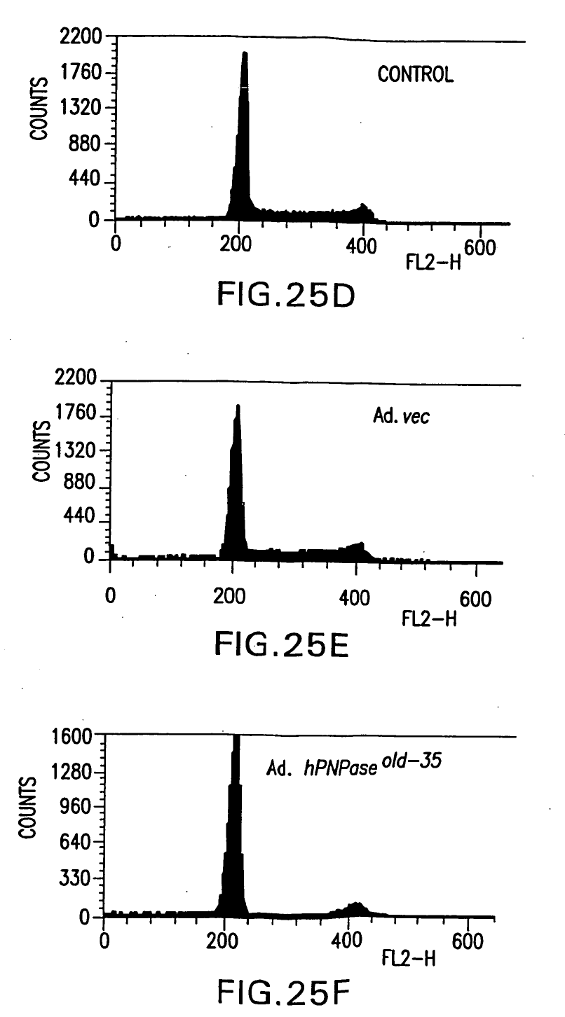

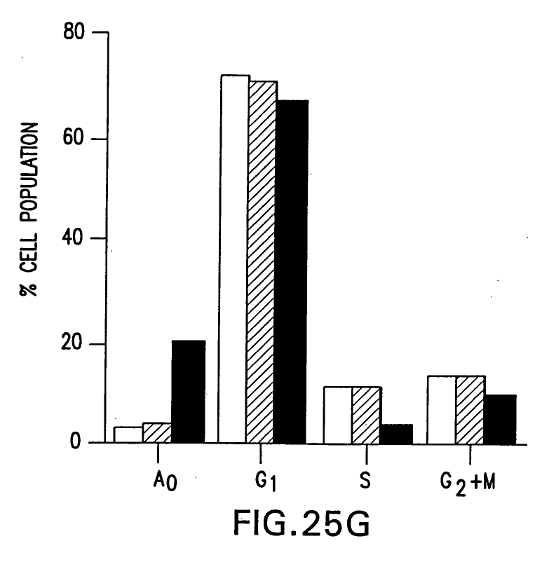

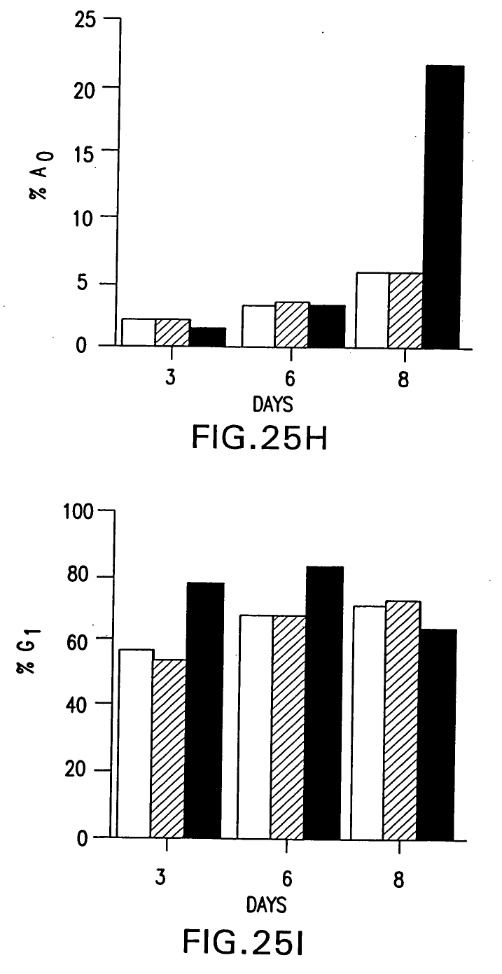

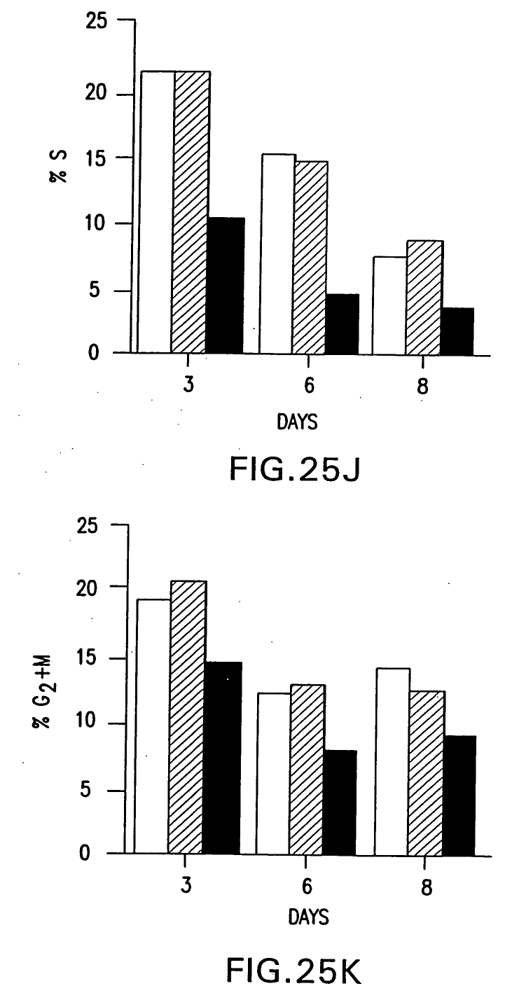

To investigate the mechanism of Ad.hPNPase^{old-35}-mediated growth inhibition, cell cycle analysis was performed following Ad.hPNPase^{old-35} infection in HO-1 cells. When the cells were infected with Ad.hPNPase^{old-35} at a high multiplicity of infection (m.o.i) of 100 pfu/cell for 4 days, there was a significant increase in sub-G0 population of cells indicating apoptosis and a decrease in the S-phase indicating inhibition of DNA synthesis (FIGS. 25A 25B). When the kinetics of killing was slowed down by infecting cells at a low m.o.i of 25 pfu/cell, there was an initial significant increase in cells in the G1 phase of the cell cycle at 3 and 6 days post-infection (FIGS. 25C and 25D). This increase was also accompanied by a marked decrease in the S-phase. At later time points, the cells infected with Ad.hPNPase^{old-35}, but not control or Ad.vec-infected cells, started to die by apoptosis (FIG. 25D). The kinetics of cell death was very slow when HO-1 cells were infected with Ad.hPNPase^{old-35} at a low m.o.i. It is worth noting that at 25 pfu/cell ˜90% of the cells are infected with adenovirus. From these observations it might be inferred that infection with Ad.hPNPase^{old-35} induces cell cycle arrest at the G1 phase that ultimately culminates in apoptosis.

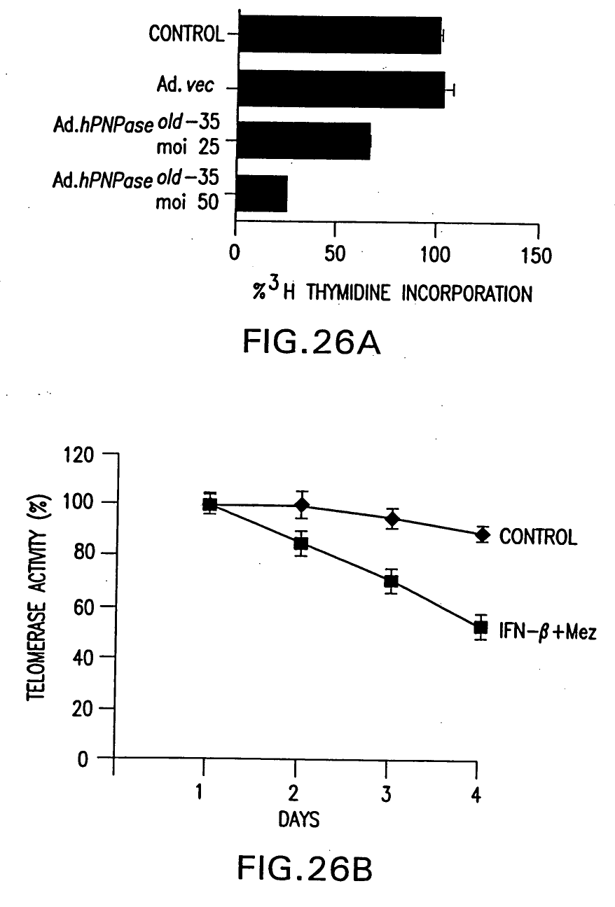

The inhibition of DNA synthesis following Ad.hPNPase^{old-35} infection was confirmed using a ³H-thymidine incorporation assay. As shown in FIG. 26A, infection with Ad.vec did not have an impact on DNA synthesis. Infection with Ad.hPNPase^{old-35} reduced DNA synthesis by ˜40% at an m.o.i of 25 pfu/cell and by ˜75% at 50 pfu/cell 4 days post-infection.

Telomerase activity is decreased in both terminal differentiation and senescence. As shown in FIG. 26B, telomerase activity decreased in a time-dependent manner to ˜50% when HO-1 cells were treated with IFN-beta+MEZ for up to 4 days. This treatment protocol results in the induction of irreversible growth arrest and terminal differentiation in HO-1 melanoma cells. Based on these findings, telomerase activity was also determined following Ad.hPNPase^{old-35} infection. As shown in FIG. 17, infection with Ad.hPNPase^{old-35} at an m.o.i of 100 pfu/cell, but not with Ad.vec, inhibited telomerase activity by almost 60% at day 4 post-infection.

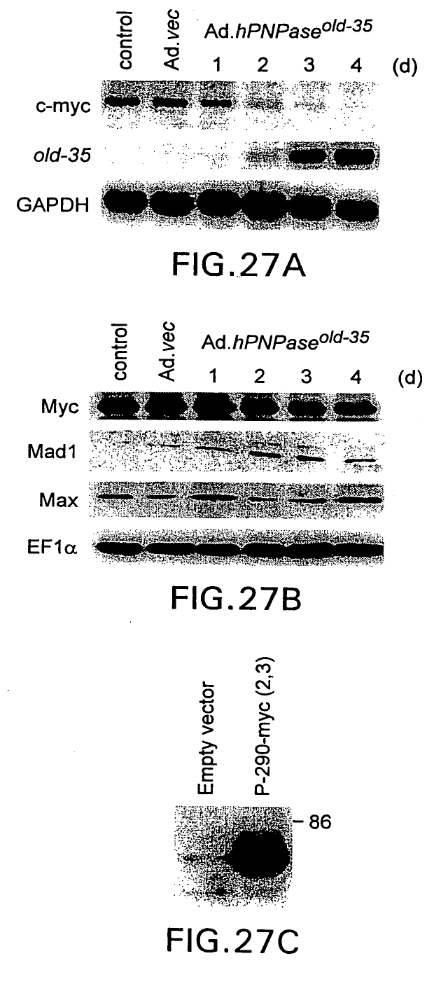

One of the factors that facilitate entry into the S-phase of the cell cycle is Myc. During terminal differentiation of melanoma cells, c-myc mRNA expression is downregulated (Jiang, et al., 1995, Oncogene 11: 1179-1189). The expression level of c-myc mRNA following Ad.hPNPase^{old-35} infection was, therefore, determined by Northern blot analysis. The expression of c-myc mRNA began decreasing 2 days post-Ad.hPNPase^{old-35}-infection but not in uninfected or Ad.vec infected cells even at 4 days post-infection (FIG. 27A). This decrease correlates with the expression of hPNPase^{old-35} mRNA that was also detected 2 days post-infection. It should be noted that under basal condition, hPNPase^{old-35} mRNA is undetectable in HO-1 cells. The expression of the housekeeping gene GAPDH remained unchanged following Ad.hPNPase^{old-35} infection.

Downregulation of Myc protein by different stimuli is usually accompanied by upregulation of Mad1, the transcriptional repressor belonging to the Max family of transcription factors (Grandori, C., Cowley, S. M., James, L. P., and Eisenman, R. N. (2000) Annu Rev Cell Dev Biol 16, 653-699). In this context, the expressions of Myc, its heterodimer partner Max and Mad1 were determined by Western blot analysis following Ad.hPNPase^{old-35} infection. As anticipated from Northern blot analysis, Myc expression started decreasing 2 days post-Ad.hPNPase^{old-35}-infection but not in uninfected or Ad.vec infected cells at 4 days post-infection (FIG. 27B). This downregulation was accompanied by upregulation of Mad1 protein. The level of Max protein remained unchanged indicating that infection with Ad.hPNPase^{old-35} switches the Myc-Max transcriptional activator to Mad1-Max transcriptional repressor. The expression level of the housekeeping gene E1alpha did not change under any condition.

We next addressed whether c-myc overexpression could protect HO-1 cells from Ad.hPNPase^{old-35}-mediated cell death. At first we determined whether the Myc expression plasmid generates the appropriate protein. For this assay, HEK-293 cells were used since transfection efficiency in these cells is very high permitting easy detection of expressed protein by Western blot analysis. As shown in FIG. 27C, transfection of p290-myc in HEK-293 cells resulted in significant overexpression of Myc in comparison to the cells transfected with empty vector. For protection assays, HO-1 cells were transfected with p290-myc, infected with Ad.hPNPase^{old-35} and the growth of the cells were analyzed by colony formation assays. Overexpression of Myc provided partial but significant protection against Ad.hPNPaseold-35 mediated cell death (FIGS. 19A and 19B), consistent with the possibility that one pathway by which Ad.hPNPaseold-35 induces growth suppression and cell death is by downregulation of Myc.

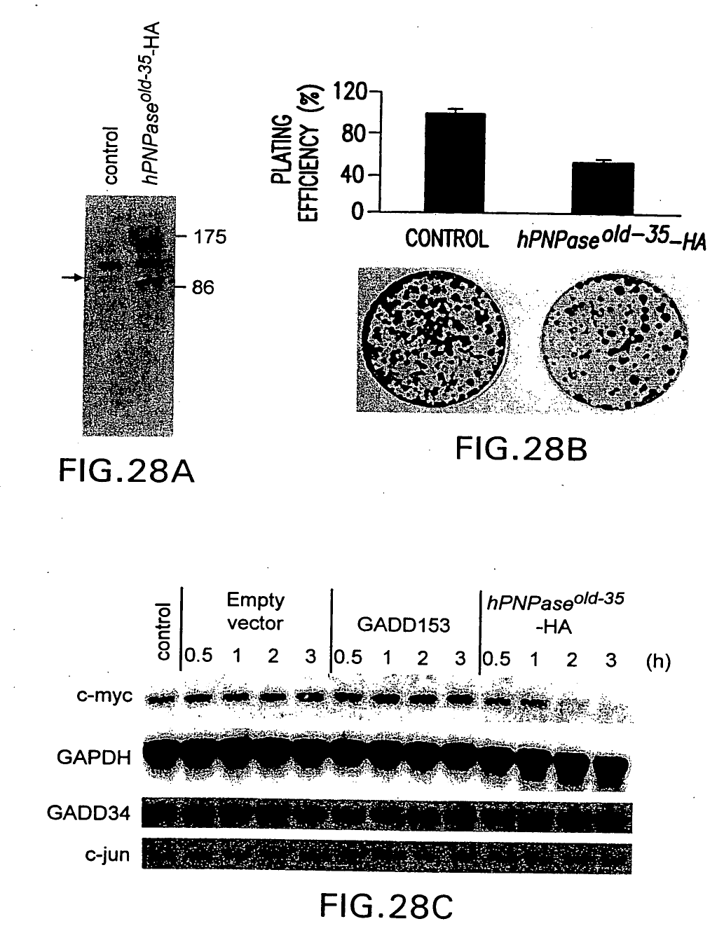

hPNPase^{old-35} is a 3′-5′ exoribonuclease prompting us to determine whether it can directly degrade c-myc mRNA. For this analysis a C-terminal HA-tagged hPNPase^{old-35} expressing construct (hPNPase^{old-35}-HA) was created. The authenticity of the construct was first confirmed by transfecting it into HEK-293 cells. As shown in FIG. 28A, Western blot analysis using Anti-HA antibody detected a single protein of ˜99 kD in size only in the hPNPase^{old-35}-HA-transfected cells. To check whether this construct has functional similarity to Ad.hPNPaseold-35, HO-1 cells were transfected with hPNPaseold-35-HA and cell growth was monitored by colony formation assay. As shown in FIG. 28B, overexpression of hPNPase^{old-35}-HA reduced colony forming ability by ˜45% indicating that hPNPase^{old-35}-HA also has growth suppressing properties. After establishing that hPNPase^{old-35}-HA generates functional protein this construct was used to prepare in vitro translated hPNPase^{OLD-35}. The effect of hPNPase^{OLD-35} protein on c-myc mRNA was investigated by in vitro mRNA degradation assays. As shown in FIG. 28C, incubation with in vitro translated hPNPase^{OLD-35} resulted in degradation of c-myc mRNA. This effect was specific for c-myc because the mRNAs for the housekeeping gene GAPDH, cell growth regulatory gene c-jun and apoptosis-inducing gene GADD34 were not degraded. This effect was also specific for hPNPase^{OLD-35} because incubation with in vitro translated GADD153, a transcription factor, did not result in mRNA degradation.

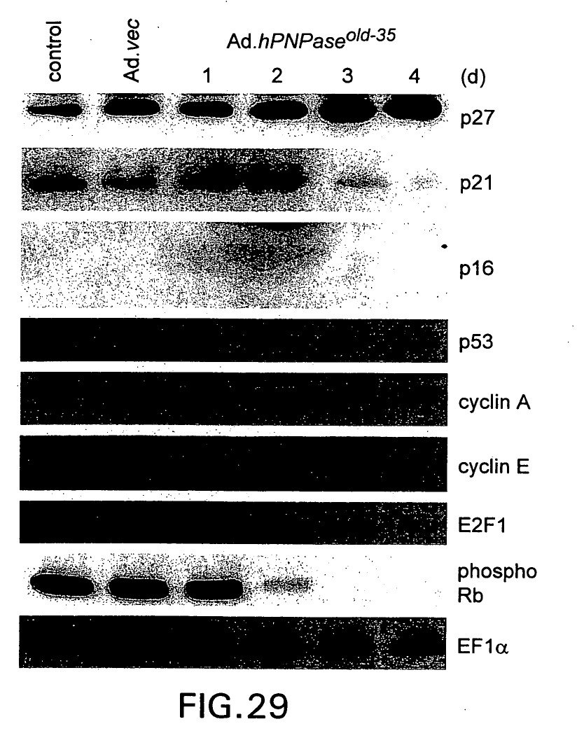

Since Ad.hPNPase^{old-35} infection reduces the S phase of the cell cycle the expression level of the regulators of G1 to S transition was checked. This checkpoint is guarded by cyclin dependent kinase inhibitors (CDKI). Ad.hPNPase^{old-35} infection resulted in progressive upregulation of p27KIP1 and the level of p21CIP1/VAF-1/MDA-6 was downregulated (FIG. 29). The expression of p16INK4A could not be detected in these cells, which is due to the fact that a majority of melanomas have genomic abnormalities in the p16INK4A gene. The expression level of p53 did not change upon Ad.hPNPase^{old-35} infection. There was a significant decrease in the levels of cyclin A, cyclin E, hyperphosphorylated form of Rb and E2F1 following Ad.hPNPase^{old-35} infection when compared to control or Ad.vec-infected cells at 4 days post-infection. The expression level of the housekeeping gene EF1alpha did not change under any condition.

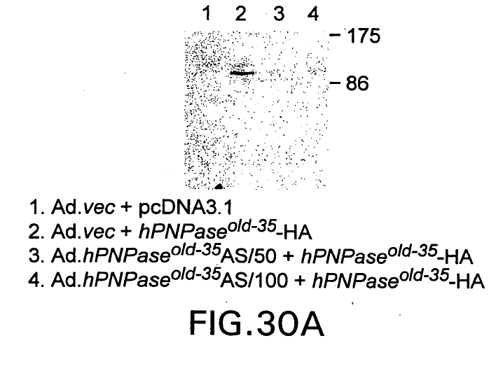

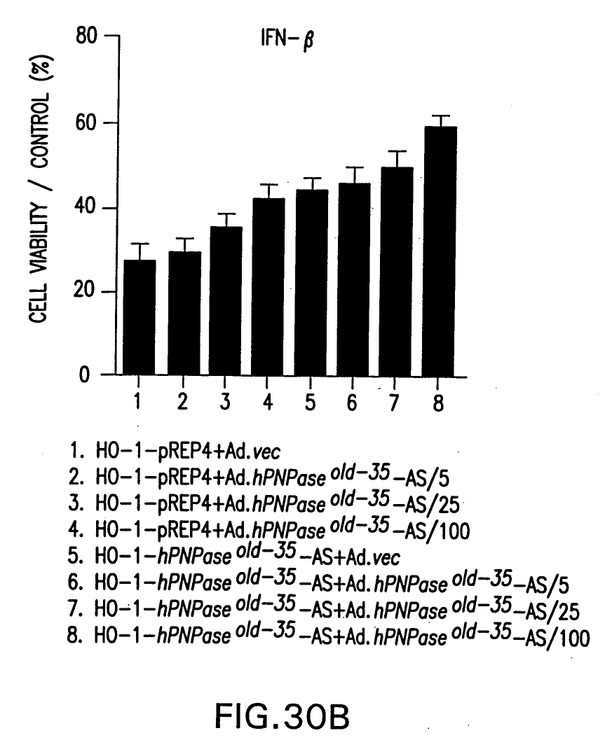

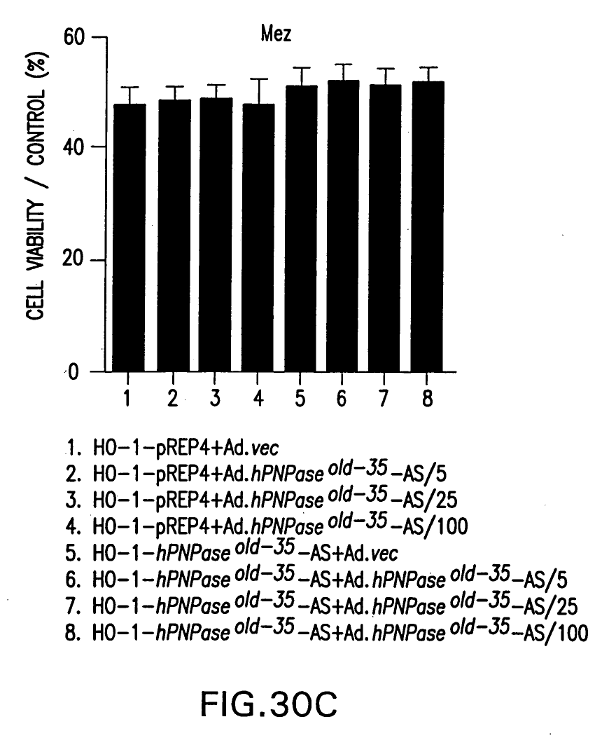

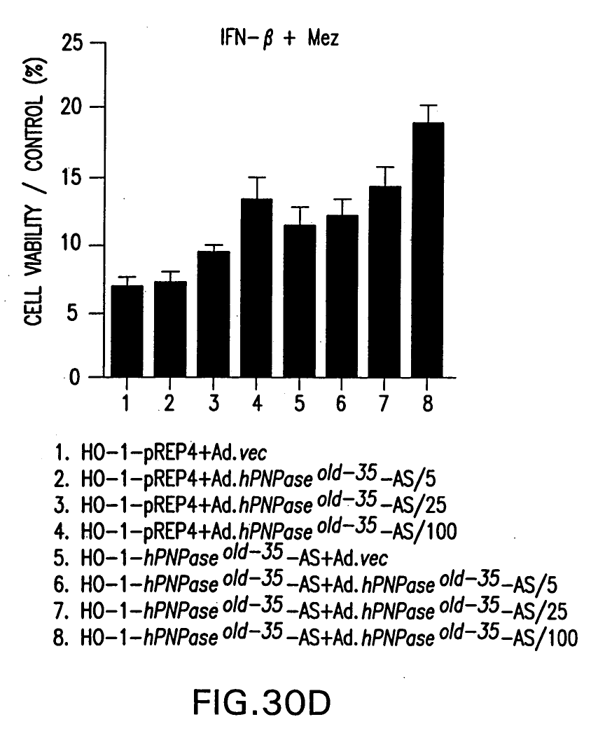

The next question that arose was the biological significance of the growth arrest mediated by hPNPase^{old-35}. Since hPNPase^{old-35} is induced during terminal differentiation by IFN-beta and MEZ we employed an antisense approach to determine the role of hPNPase^{old-35} in the growth arrest associated with treatment with IFN-beta, MEZ or IFN-beta+MEZ. Ad.hPNPase^{old-35}AS was constructed and evaluated for activity. HO-1 cells were transfected with hPNPase^{old-35}-HA, followed by infection with Ad.hPNPase^{old-35}AS. The expression of hPNPase^{OLD-35}-HA was detected in the cell lysates by Western blot analysis using Anti-HA antibody. As shown in FIG. 30A, hPNPase^{OLD-35}-HA could be detected in the cells following hPNPase^{old-35}-HA transfection (lane 2). However, infection with Ad.hPNPaseold-35AS at an m.o.i. of 50 or 100 pfu/cell resulted in marked reduction of hPNPaseOLD-35-HA (lanes 3 and 4) suggesting that Ad.hPNPase^{old-35}AS could effectively inhibit the expression of hPNPase^{OLD-35}. We generated a cell line in the HO-1 background that stably overexpresses hPNPase^{old-35}AS (HO-1-hPNPase^{old-35}AS). HO-1-pREP4 and HO-1-hPNPaseold-35AS cells were infected with Ad.hPNPase^{old-35}AS at an m.o.i. of 5, 25 and 100 pfu/cell and after 24 h the cells were treated with either IFN-beta or MEZ, alone or in combination. Cell viability was assayed after 5 days. It should be noted that HO-1 cells are relatively refractory to IFN-beta and a high concentration of IFN-beta (2000 units/ml) is required to induce growth inhibition (Fisher, et al., 1985, J Interferon Res 5: 11-22; Jiang, et al., 1993, Mol Cell Different 1: 41-662). Infection with Ad.hPNPase^{old-35}AS provided small but significant protection against IFN-beta-induced growth inhibition both in HO-1-pREP4 and in HO-1-hPNPaseold-35AS in a dose-dependent manner (FIGS. 30B and 30D). Treatment with IFN-beta reduced cell viability by ˜75% in HO-1-pREP4. In HO-1-pREP4 infected with Ad.hPNPase^{old-35}AS at an m.o.i. of 100 pfu/cell and in HO-1-hPNPaseold-35AS cell viability was reduced by ˜55% by IFN-beta (FIG. 30B, columns 4 and 5, respectively). When HO-1-hPNPase^{old-35}AS cells were infected with Ad.hPNPase^{old-35}AS at an m.o.i. of 100 pfu/cell the cell viability was reduced by only ˜45% (FIG. 30B, column 8). A combination of IFN-beta and MEZ treatment reduced cell viability of HO-1-pREP4 by ˜94% (FIG. 30D, column 1). This inhibitory effect was partially reversed in HO-1-pREP4 cells infected with Ad.hPNPase^{old-35}AS at an m.o.i. of 100 pfu/cell and in HO-1-hPNPase^{old-35}AS cells, the reduction in cell viability was ˜87% (FIG. 30D, columns 4 and 5, respectively). In HO-1-hPNPase^{old-35}AS cells infected with Ad.hPNPase^{old-35}AS at an m.o.i. of 100 pfu/cell the reduction in cell viability was ˜80% (FIG. 30D, column 8). The antisense approach did not provide protection against MEZ induced growth arrest (FIG. 30C) even at an m.o.i. of 100 pfu/cell. Because of non-specific cytotoxicities of adenovirus infection a m.o.i. higher than 100 pfu/cell was not employed in these studies. These results indicate that as a predominantly type I interferon-inducible gene hPNPase^{old-35} might be involved in mediating growth arrest induced by IFN-beta. The observation that infection of Ad.hPNPase^{old-35}AS in HO-1-hPNPase^{old-35}AS cells provided superior protection indicates that the dosage of antisense molecules might determine the relative level of protection from growth inhibition.

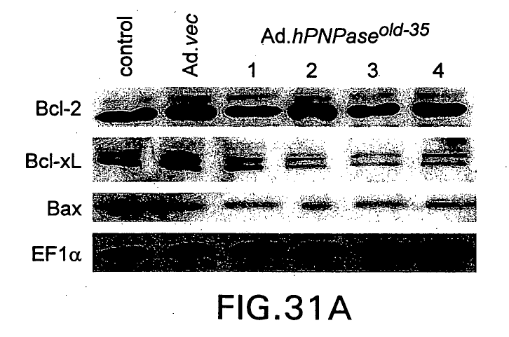

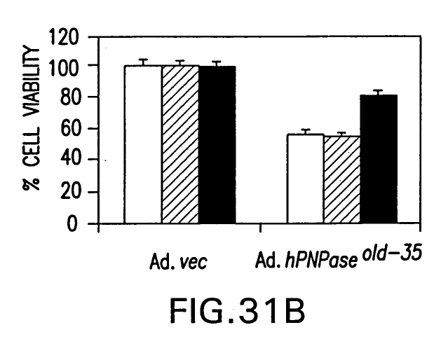

Since Ad.hPNPase^{old-35} infection induces apoptosis in HO-1 cells, the effect of Ad.hPNPase^{old-35} infection on the expression levels of pro- and anti-apoptotic genes were examined. Infection with Ad.hPNPase^{old-35} resulted in downregulation of the anti-apoptotic protein Bcl-xL (FIG. 31A). The expression levels of the anti-apoptotic protein Bcl-2 and pro-apoptotic protein Bax remained unchanged. Stable HO-1 cell lines expressing either Bcl-2 or Bcl-xL were generated and these cell lines were infected with Ad.hPNPase^{old-35}. As shown in FIG. 31B, overexpression of Bcl-xL, but not Bcl-2, provided partial protection against Ad.hPNPase^{old-35} induced cell death.

Various publications are cited herein, the contents of which are hereby incorporated by reference in their entireties.

We claim:

1. An isolated nucleic acid molecule encoding a protein having a sequence as set forth in SEQ ID NO:2.

2. An isolated nucleic acid molecule comprising a nucleic acid encoding a protein having a sequence as set forth in SEQ ID NO:2 operatively linked to a promoter element.

3. The isolated nucleic acid molecule of claim 2, wherein the promoter is an old-35 promoter.

4. The isolated nucleic acid molecule of claim 3, wherein the old-35 promoter has a sequence as set forth in SEQ ID NO:4.

5. The isolated nucleic acid molecule of claim 3, wherein the old-35 promoter is the p400 variant.

6. The isolated nucleic acid molecule of claim 3, wherein the promoter is a heterologous promoter.

7. The nucleic acid molecule of claim 2, as contained in a vector molecule.

8. The isolated nucleic acid molecule of claim 1, wherein the nucleic acid molecule encoding a protein having a sequence as set forth in SEQ ID NO:2 has a nucleotide sequence as set forth in SEQ ID NO:1.

9. The isolated nucleic acid molecule of claim 2, wherein the nucleic acid molecule encoding a protein having a sequence as set forth in SEQ ID NO:2 has a nucleotide sequence as set forth in SEQ ID NO:1.

10. The isolated nucleic acid molecule of claim 3, wherein the nucleic acid molecule encoding a protein having a sequence as set forth in SEQ ID NO:2 has a nucleotide sequence as set forth in SEQ ID NO:1.

11. The isolated nucleic acid molecule of claim 4, wherein the nucleic acid molecule encoding a protein having a sequence as set forth in SEQ ID NO:2 has a nucleotide sequence as set forth in SEQ ID NO:1.

12. The isolated nucleic acid molecule of claim 5, wherein the nucleic acid molecule encoding a protein having a sequence as set forth in SEQ ID NO:2 has a nucleotide sequence as set forth in SEQ ID NO:1.

13. The isolated nucleic acid molecule of claim 6, wherein the nucleic acid molecule encoding a protein having a sequence as set forth in SEQ ID NO:2 has a nucleotide sequence as set forth in SEQ ID NO:1.

14. The isolated nucleic acid molecule of claim 7, wherein the nucleic acid molecule encoding a protein having a sequence as set forth in SEQ ID NO:2 has a nucleotide sequence as set forth in SEQ ID NO:1.

15. An isolated protein comprising an amino acid sequence as set forth in SEQ ID NO:2.

16. A method of promoting terminal differentiation in a cell comprising introducing, into the cell, an old-35 nucleic acid operatively linked to a promoter element, such that an amount of OLD-35 protein is produced sufficient to increase at least one indicia of terminal differentiation.

17. A method of promoting senescence in a cell comprising introducing, into the cell, an old-35 nucleic acid operatively linked to a promoter element, such that an amount of OLD-35 protein is produced sufficient to increase at least one indicia of senescence.

18. A method of reversing a transformed phenotype of a cell comprising introducing, into the cell, an old-35 nucleic acid operatively linked to a promoter element, such that an amount of OLD-35 protein is produced sufficient to decrease at least one indicia of the transformed phenotype.

19. A method of decreasing the rate of cell proliferation comprising introducing, into the cell, an old-35 nucleic acid operatively linked to a promoter element, such that an amount of OLD-35 protein is produced sufficient to decrease the rate of cell proliferation.

20. A method of promoting terminal differentiation in a cell comprising introducing, into the cell, an amount of OLD-35 protein sufficient to increase at least one indicia of terminal differentiation.

21. A method of promoting senescence in a cell comprising introducing, into the cell, an amount of OLD-35 protein sufficient to increase at least one indicia of senescence.

22. A method of reversing a transformed phenotype of a cell comprising introducing, into the cell, an amount of OLD-35 protein sufficient to decrease at least one indicia of the transformed phenotype.

23. A method of decreasing the rate of cell proliferation comprising introducing, into the cell, an amount of OLD-35 protein sufficient to decrease the rate of cell proliferation.

24. An isolated nucleic acid comprising an old-35 promoter having a nucleic acid sequence as set forth in SEQ ID NO:4.

25. An isolated nucleic acid comprising an old-35 promoter which hybridizes to a nucleic acid molecule having a sequence as set forth in SEQ ID NO:4, or its complementary strand, under stringent conditions.

26. An isolated nucleic acid molecule comprising an old-35 promoter, which is p2000 as depicted in FIG. 5A.

27. An isolated nucleic acid molecule comprising an old-35 promoter, which is p1000 as depicted in FIG. 5A.

28. An isolated nucleic acid comprising an old-35 promoter, which is p400 as depicted in FIG. 5A.

29. An isolated nucleic acid comprising an old-35 promoter, which is p2000/−400 as depicted in FIG. 5A.

30. An isolated nucleic acid comprising an old-35 promoter, which is p400/−60 as depicted in FIG. 5A.

31. An isolated nucleic acid comprising an old-35 promoter, which is p400-mISRE as depicted in FIG. 5A and comprising a nucleic acid having a sequence as set forth in SEQ ID NO:7.

32. A nucleic acid molecule comprising an old-35 promoter operatively linked to a gene wherein the gene is not an old-35 gene.

33. An isolated nucleic acid molecule comprising an old-35 promoter operatively linked to a nucleic acid which encodes an OLD-35 protein.

34. A cell containing an old-35 promoter operatively linked to a heterologous gene of interest.

35. The cell of claim 34, wherein the old-35 promoter comprises a nucleic acid molecule having a nucleotide sequence as set forth in SEQ ID NO:4.

36. The cell of claim 34, wherein the old-35 promoter hybridizes to a nucleic acid molecule having a sequence as set forth in SEQ ID NO:4, or its complementary strand, under stringent conditions.

37. The cell of claim 34, wherein the old-35 promoter is p2000 as depicted in FIG. 5A.

38. The cell of claim 34, wherein the old-35 promoter is p1000 as depicted in FIG. 5A.

39. The cell of claim 34, wherein the old-35 promoter is p400 as depicted in FIG. 5A.

40. The cell of claim 34, wherein the old-35 promoter is p2000/−400 as depicted in FIG. 5A.

41. The cell of claim 34, wherein the old-35 promoter is p400/−60 as depicted in FIG. 5A.

42. The cell of claim 34, wherein the old-35 promoter is p400-mISRE as depicted in FIG. 5A and comprising a nucleic acid having a sequence as set forth in SEQ ID NO:7.

43. An assay system comprising the cell of claim 34, wherein if the gene of interest is transcribed, a detectable product is produced.

44. The cell of claim 34, wherein the gene of interest is a reporter gene.

45. A assay method comprising exposing a cell containing an old-35 promoter linked to a gene of interest to a test agent and determining the effect of the test agent on the production of a product of the gene of interest.

46. The assay method of claim 45, wherein the product is a direct product.

47. The assay method of claim 46, wherein an increase in the level of product correlates positively with the ability of the test agent to increase old-35 promoter activity.

48. The assay method of claim 45, wherein the old-35 promoter comprises a nucleic acid molecule having a nucleotide sequence as set forth in SEQ ID NO:4.

49. The assay method of claim 45, wherein the old-35 promoter hybridizes to a nucleic acid molecule having a sequence as set forth in SEQ ID NO:4, or its complementary strand, under stringent conditions.

50. The assay method of claim 45, wherein the old-35 promoter is p2000 as depicted in FIG. 5A.

51. The assay method of claim 45, wherein the old-35 promoter is p1000 as depicted in FIG. 5A.

52. The assay method of claim 45, wherein the old-35 promoter is p400 as depicted in FIG. 5A.

53. The assay method of claim 45, wherein the old-35 promoter is p2000/−400 as depicted in FIG. 5A.

54. The assay method of claim 45, wherein the old-35 promoter is p400/−60 as depicted in FIG. 5A.

55. The assay method of claim 45, wherein the old-35 promoter is p400-mISRE as depicted in FIG. 5A and comprising a nucleic acid having a sequence as set forth in SEQ ID NO:7.
